(19) United States
(12) Patent Application Publication
(10) Pub. No.: US 2018/0044736 A1
(43) Pub. Date: Feb. 15, 2018

(54) BIOMARKER BASED PROGNOSTIC MODEL FOR PREDICTING OVERALL SURVIVAL IN PATIENTS WITH METASTATIC CLEAR CELL KIDNEY CANCER

(71) Applicants: Cedars-Sinai Medical Center, Los Angeles, CA (US); Duke University, Durham, NC (US)

(72) Inventors: Hyung KIM, Los Angeles, CA (US); Ping LI, Los Angeles, CA (US); Susan HALABI, Durham, NC (US)

(73) Assignees: Cedars-Sinai Medical Center, Los Angeles, CA (US); Duke University, Durham, NC (US)

(21) Appl. No.: 15/548,010

(22) PCT Filed: Feb. 3, 2016
(86) PCT No.: PCT/US16/16460

§ 371 Date: Aug. 1, 2017

Related U.S. Application Data
(60) Provisional application No. 62/111,569, filed on Feb. 3, 2015.

(51) Int. Cl. C12Q 1/68

(52) U.S. Cl. CPC C12Q 1/6886; C12Q 2600/118; C12Q 2600/158; C12Q 2600/106

(57) ABSTRACT

The present invention describes a method of using a molecular prognostic signature, to predict overall survival in patients with metastatic clear cell renal cell carcinoma. The present invention also describes a method of selecting therapy and a process for patient risk stratification based on the molecular prognostic signature analysis.

### STATEMENT REGARDING FEDERALLY-SPONSORED RESEARCH

This invention was made with government support under Grant Nos. CA133072, CA155296 and CA157703 awarded by the National Institutes of Health. The government has certain rights in the invention.

### FIELD OF INVENTION

This invention relates to renal cell carcinoma.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Most localized renal cell carcinomas (RCCs) have a favorable prognosis, and the American Urologic Association recommends observation as a valid management for many small renal cancers. In contrast, metastatic RCC is nearly always fatal. Despite being uniformly fatal, survival associated with metastatic RCC can vary widely, from a few months to several years. While clinical findings and histomorphologic characteristics of the tumor can provide reasonable estimates of survival, greater precision is needed. However, molecular signatures from the primary tumor promise to provide more accurate prognosis, which is useful for patient counseling, treatment planning, determining clinical trial eligibility and comparing results between trials.

Many prior studies have reported biomarkers and molecular signatures for predicting survival (Takahashi et al., 2001, Sultmann et al., 2005, Kosari et al., 2005, Jones et al., 2005, Zhao et al., 2006, Cancer Genome Atlas Research N, 2013, Brannon et al., 2010). Unfortunately, the majority of these studies included patients with both localized and metastatic RCC. There is a lack of studies reporting prognostic molecular signatures that can be applied to metastatic RCC. Such signatures can be generated from primary tumors sampled during diagnostic biopsy or cytoreductive nephrectomy, which remains an important standard-of-care. In RCC, two separate phase III trials have shown that cytoreductive nephrectomy improves survival in patients treated with cytokine therapy. In patients receiving more modern targeted therapies, retrospective studies suggest a survival benefit for cytoreductive surgery. Therefore, it is not surprising that molecular signatures can be readily identified for separating these patients into two prognostic groups.

Prior studies of biomarkers from cytoreductive nephrectomies are also limited by small sample sizes and have usually focused on a limited numbers of candidate markers assessed by immunohistochemistry (Vasselli et al., 2003, Miyake et al., 2009, Kusuda et al., 2013, Kim et al., 2005). To develop prognostic biomarkers for metastatic RCC, there remains a need for discovery studies using multi-institutional tissue banks from well-characterized patients whose treatment and outcomes were rigorously annotated. Therefore, the inventors disclose a gene expression-based prognostic signature developed using primary untreated RCC collected as part of Cancer and Leukemia Group B (CALGB) 90206, a randomized phase III trial of interferon alpha (INF) vs. INF plus bevacizumab in patients with metastatic or unresectable RCC (Rini et al., 2010, J. Clin. Oncol. 28 (13), 2137-2143). CALGB is now a part of the Alliance for Clinical Trials in Oncology. CALGB 90206 was used to develop a prognostic signature for predicting OS as described herein.

Approximately one third of patients newly diagnosed with RCC have metastatic disease, and after treatment for localized RCC, 25-50% of patients will suffer recurrence. The prognosis associated with recurrent and metastatic RCC is poor.

Therefore, there is a need in the art for methods for determining overall survival and patient stratification using molecular prognostic signatures and for selecting therapy and/or treatment for these patients.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of determining overall survival in a subject with metastatic clear cell renal cell carcinoma, comprising: obtaining a biological sample from the subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a coefficient or using a calculated coefficient for each metastatic clear cell renal cell carcinoma gene; and determining that the subject has a good overall survival if the coefficient is negative with a low gene expression level or if the coefficient is positive with a high gene expression level and determining that the subject has poor overall survival if the coefficient is negative with a high gene expression level or if the coefficient is positive with a low gene expression level.

Various other embodiments of the present invention provide for a method of determining overall survival in a subject with metastatic clear cell renal cell carcinoma, comprising: obtaining a biological sample from the subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a hazard ratio or using a calculated hazard ratio for each metastatic clear cell renal cell carcinoma gene; calculating a risk score from the hazard ratio; and stratifying the subject into a low, intermediate and high risk group for metastatic clear cell renal cell carcinoma from the risk score; wherein a subject in a low risk group has a good overall survival, a subject in an intermediate risk group has an intermediate overall survival and the subject in a high risk group has a poor overall survival.

Various embodiments of the present invention also provide for a process of patient risk stratification, comprising: obtaining a biological sample from a subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a coefficient or using a calculated coefficient for each metastatic clear cell renal cell carcinoma gene; calculating a risk score from the coefficient; and stratifying the subject into risk groups for metastatic clear cell renal cell carcinoma from the risk score.

Various other embodiments of the present invention also provide for a method of selecting a therapy and/or treatment for metastatic renal cell carcinoma, comprising: obtaining a biological sample from a subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a coefficient or using a calculated coefficient for each metastatic clear cell renal cell carcinoma gene; calculating a risk score from the coefficient; stratifying the subject into a low, intermediate and high risk group for metastatic clear cell renal cell carcinoma from the risk score; wherein a subject in a low risk group has a good overall survival, a subject in an intermediate risk group has an intermediate overall survival and the subject in a high risk group has a poor overall survival; and selecting a first therapy for a subject in the low, intermediate and high risk group, selecting a second therapy for a subject in the low or intermediate risk group, and selecting a third therapy, or a combination of the first, second and third therapy for a subject in a high risk group.

In various embodiments, the first therapy can be selected from the group consisting of surgical resection, radical or partial nephrectomy, active surveillance, palliative radiation therapy, metastasectomy and/or bisphonates. In various other embodiments, the second therapy can be a targeted therapy drug or immunotherapy. In yet other embodiments, the targeted therapy drug can be selected from the group consisting of VEGF inhibitors or mTOR inhibitors. In certain embodiments, the VEGF inhibitors can be selected from the group consisting of Sunitinib, Pazopanib, Bevacizumab, Sorafenib, Axitinib, and combinations thereof. In certain other embodiments, the mTOR inhibitors can be selected from the group consisting of Temsirolimus, Everolimus, and combinations thereof. In yet other embodiments, the immunotherapy can be selected from the group consisting of high-dose Interleukin-2, low-dose Interleukin-2, Interferon-alpha 2a or combinations thereof. In various other embodiments, the third therapy can be thermal ablation, a combination of the first and second therapy, and combinations thereof. In some embodiments, thermal ablation can be cryoablation and radiofrequency ablation.

Other embodiments of the present invention also provide for a method of selecting a metastatic clear cell renal cell carcinoma subject for a clinical trial, comprising: obtaining a biological sample from a subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a coefficient or using a calculated coefficient for each metastatic clear cell renal cell carcinoma gene; calculating a risk score from the coefficients; stratifying the subject into a low, intermediate and high risk group for metastatic clear cell renal cell carcinoma from the risk score; wherein a subject in a low risk group has a good overall survival, a subject in an intermediate risk group has an intermediate overall survival and the subject in a high risk group has a poor overall survival; and selecting the subject for a clinical trial if the subject falls within the appropriate risk group for the clinical trial, wherein a subject in a low risk group is selected for a low risk group clinical trial, a subject in an intermediate risk group is selected for an intermediate risk group clinical trial and a subject in a high risk group is selected for a high risk group clinical trial.

Various other embodiments of the present invention provide for a method of determining overall survival in a subject with metastatic clear cell renal cell carcinoma, comprising: obtaining a biological sample from a subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; and determining that the subject has a good overall survival if there is an increased expression level of CRYL1, PCNA, CDK1, or a combination thereof, or if there is an decreased expression level of TRAF2, USP6NL, CEP55, HGF, HSD17B10 or a combination thereof, and determining that the subject has poor overall survival if there is an decreased expression level of CRYL1, PCNA, CDK1, or a combination thereof, or if there is an increased expression level of TRAF2, USP6NL, CEP55, HGF, HSD17B10 or a combination thereof.

The methods and process described herein can comprise the following embodiments. In various embodiments, the metastatic clear cell renal cell carcinoma gene and reference gene expression level can be determined using quantitative polymerase chain reaction (qPCR), using a specific primer sequence and/or a probe sequence. In various other embodiments, the primer sequence or probe sequence used for the metastatic clear cell renal cell carcinoma gene and the reference gene can be: CDK1: ACCTATGGAGTTGTGTATAAGGGTAGAC (SEQ ID NO: 1), ACCCCTTCCTCTTCACTTTCTAGT (SEQ ID NO: 2) and CATGGCTACCACTTGACC (SEQ ID NO: 3); CEP55: CTCCAAACTGCTTCAACTCATCAAT (SEQ ID NO: 4), ACACGAGCCACTGCTGATTTT (SEQ ID NO: 5) and CTCCAGAGCATCTTTC (SEQ ID NO: 6); CRYL1: CGTTGGCAGTGGAGTCATTG (SEQ ID NO: 7), GGAAGCCTCCACTGGCAAA (SEQ ID NO: 8) and ATGGCCCAGCTTCGCC (SEQ ID NO: 9); HGF: CATTCACTTGCAAGGCTTTTGTTTT (SEQ ID NO: 10), TTTCACTCCACTTGACATGCTATTGA (SEQ ID NO: 11) and AACAATGCCTCTGGTTCCC (SEQ ID NO: 12); HSD17B10: CCAAGCCAAGAAGTTAGGAAACAAC (SEQ ID NO: 13), GCTGTTTGCACATCCTTCTCAGA (SEQ ID NO: 14) and CCCAGCCGACGTGACC (SEQ ID NO: 15); PCNA: TGAACCTCACCAGTATGTCCAAAAT (SEQ ID NO: 16), CGTTATCTTCGGCCCTTAGTGTAAT (SEQ ID NO: 17) and CCGGCGCATTTTAGT (SEQ ID NO: 18); TRAF2: GGAAGCGCCAGGAAGCT (SEQ ID NO: 19), CCGTACCTGCTGGTGTAGAAG (SEQ ID NO: 20) and ATACCCGCCATCTTCT (SEQ ID NO: 21); USP6NL: GAGGAGCTCCCAGATCATAATGTG (SEQ ID NO: 22), GCATTTTCAGCCATTTGGTAGTTCT (SEQ ID NO: 23) and AAGCACCTGGAAATTG (SEQ ID NO: 24); ACTB: CCAGCTCACCATGGATGATG (SEQ ID NO: 25), ATGCCGGAGCCGTTGTC (SEQ ID NO: 26) and TCGCCGCGCTCGTC (SEQ ID NO: 27); GUSB: CTCATTTGGAATTTTGCCGATT (SEQ ID NO: 28), CCGAGTGAAGATCCCCTTTTTA (SEQ ID NO: 29) and TCACCGACGAGAGTGC (SEQ ID NO: 30); HPRT1: ATGGACAGGACTGAACGTCTTG (SEQ ID NO: 31), GCACACAGAGGGCTACAATGT (SEQ ID NO: 32) and CCTCCCATCTCCTTCATCA (SEQ ID NO: 33); RPL13A: ACCAACCCTTCCCGAGGC (SEQ ID NO: 34), TTGGTTTTGTGGGGCAGCAT (SEQ ID NO: 35) and ACGGTCCGCCAGAAGA (SEQ ID NO: 36); RPLP0: CCACGCTGCTGAACATGCT (SEQ ID NO: 37), TCGAACACCTGCTGGATGAC (SEQ ID NO: 38) and TCTCCCCCTTCTCCTTTG (SEQ ID NO: 39) and SDHA: AGGAATCAATGCTGCTCTGGG (SEQ ID NO: 40), GTCGGAGCCCTTCACGGT (SEQ ID NO: 41) and CCACCTCCAGTTGTCC (SEQ ID NO: 42).

In certain embodiments, the calculated coefficient for the metastatic clear cell renal cell carcinoma gene CDK1 can be 0.089, CEP55 can be −0.258, CRYL1 can be 0.356, HGF can be −0.086, HSD17B10 can be −0.232, PCNA can be 0.155, TRAF2 can be −0.215 and USP6NL can be −0.090. In other embodiments, the calculated hazard ratio for the metastatic clear cell renal cell carcinoma gene CDK1 can be 1.093, CEP55 can be 0.772, CRYL1 can be 1.428, HGF can be −0.918, HSD17B10 can be 0.793, PCNA can be 1.167, TRAF2 can be 0.806 and USP6NL can be 0.914. In some embodiments, the metastatic clear cell renal cell carcinoma gene and reference gene expression level can be determined by RNAseq, microarray and/or nanostring.

In yet other embodiments, the methods and/or process can further comprise using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in determining overall survival, aid in stratifying the patient into a risk group, to aid in selecting a therapy and/or treatment and aid in selecting a subject for a clinical trial. In some embodiments, a coefficient can be calculated for the one or more MSKCC adverse clinical risk factors. In other embodiments, the MSKCC adverse clinical risk factor coefficient for 1 and/or 2 MSKCC adverse clinical risk factors can be 0.276 or the coefficient for 3 or more MSKCC adverse clinical risk factors can be 0.954. In certain other embodiments, the coefficient can be calculated from the slope of a multi-variant regression model. In yet other embodiments, the methods and/or process can further comprise assaying the biological sample to determine an expression level for one or more of the 416 additional clear cell renal cell carcinoma genes.

In various embodiments, stratifying the subject into risk groups can comprise stratifying the subject into a low, intermediate and high risk group for metastatic clear cell renal cell carcinoma from the risk score; wherein a subject in a low risk group has a good overall survival, a subject in an intermediate risk group has an intermediate overall survival and the subject in a high risk group has a poor overall survival. In various other embodiments, patient counseling can be given to a subject that has been stratified into a low, intermediate or high risk group.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a REMARK diagram, in accordance with various embodiments of the present invention. The diagram accounts for each patient in the parent clinical trial and the availability of their tumor tissue for this study.

FIG. 2 depicts the evaluation of tumor heterogeneity. The tumor was sampled in two separate areas and gene expressions were determined by qPCR. Heterogeneity was defined as the median of standard deviations determined from sampling each tumor twice. A threshold of 0.78 for unacceptable heterogeneity (black circles to the right of 0.78) was determined using the K-means clustering algorithm with k=2.

FIG. 3 depicts the Kaplan-Meier survival curves for 8-gene-only prognostic model for OS, in accordance with various embodiments of the present invention. Multivariable model developed using the training set was used to assign risk scores to the testing set. Cutoffs for risk groups were defined by dividing the training set into tertiles. Using the test set shows a KM plot (3 groups based on risk score).

FIG. 4 depicts the Kaplan-Meier survival curves for MSKCC only prognostic model for OS, in accordance with various embodiments of the present invention. Using the test set shows a KM plot (3 groups based on risk score). Risk groups defined by number of MSKCC clinical risk factors.

FIG. 5 depicts the Kaplan-Meier survival curves final model for 8-genes plus MSKCC clinical risk factors prognostic model for OS, in accordance with various embodiments of the present invention. Using the test set shows a KM plot (3 groups based on risk score). Multivariable model developed using the training set was used to assign risk scores to the testing set. Cutoffs for risk groups were defined by dividing the training set into tertiles.

FIG. 6 depicts in accordance with various embodiments of the present invention, Kaplan-Meier survival curves for each gene in the final model (Campbell et al., 2009), by high/low expression using the entire cohort (training and testing set). The median gene expression from the training set was the cutoff used to define high/low expression. (High—solid line; low—dashed line.)

FIG. 7 depicts AUC plots at 18-months (a) and 24 months (a), in accordance with various embodiments of the present invention. The dotted line represents the final model, solid line is the model with 8 genes, and the dashed line is the MKSCC clinical risk factors only.

FIG. 8 depicts calibration plots for the final model (8 genes plus MSKCC clinical risk factors) in the training set at 18- and 24-months, in accordance with various embodiments of the present invention. (Bold solid line is observed data, dotted line is ideal and thin solid line is optimism corrected).

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Good overall survival”, as used herein means the category of patients whose prospect of survival is higher than the average survival rate of metastatic renal cell carcinoma patients (For example, at least one standard deviation higher, or in the 60-100th percentile of metastatic renal cell carcinoma patients, or in the top tertile of metastatic renal cell carcinoma patients).

“Intermediate overall survival”, as used herein means the category of patients whose prospect of survival is the average survival rate of metastatic renal cell carcinoma patients (For example, between one standard deviation above and one standard deviation below, or in the 40-60 percentile of metastatic renal cell carcinoma patients, or in the middle tertile of metastatic renal cell carcinoma patients).

“Poor overall survival”, as used herein means the category of patients whose prospect of survival is lower than the average survival rate of metastatic renal cell carcinoma patients (For example, at least one standard deviation lower, or in the 0-40 percentile of metastatic renal cell carcinoma patients, or in the bottom tertile of metastatic renal cell carcinoma patients).

Non-limiting examples of “Biological sample” include whole blood, plasma, serum, saliva, cheek swab, RCC tissue or cells or other bodily fluid or tissue.

“Multi-variable-Regression Model” as used herein means a statistical process for predicting outcome based on multiple variables.

“Patient Risk Stratification” as used herein means the process of separating patient populations into risk groups.

A “Coefficient” as used herein is a calculated number which relates to the multi-variable-regression model obtained from the gene expression levels. It depicts the relative importance/contribution of the gene in the risk score and prediction of overall survival. In various embodiments, it is the slope of the multi-variable-regression model.

A “Risk Score” as used herein is a calculated number, generated from the calculated coefficient and can be used to stratify patients into risk groups and predict survival.

“Risk Group” as used herein refers to a subset of patients who fall within the same category for overall survival. Examples of Risk Groups include but are not limited to a Risk Group based on the determined gene expression levels obtained from the patients' biological sample, and a Risk Group based on the determined gene expression levels obtained from the patients' biological sample and MSKCC adverse clinical risk factors.

The term “MSKCC” refers to the Memorial Sloan Kettering Cancer Center adverse clinical risk factors.

Selecting therapy and/or treatment as used herein, includes but is not limited to selecting, choosing, prescribing, advising, recommending, instructing, or counseling the subject with respect to treatment.

“Treatment”, as used herein refer to both therapeutic treatment and prophylactic or preventative measures, wherein the object is to prevent or slow down (lessen) the targeted pathologic condition, prevent the pathologic condition, pursue or obtain good overall survival, or lower the chances of the individual developing the condition even if the treatment is ultimately unsuccessful. Those in need of treatment include those already with the condition as well as those prone to have the condition or those in whom the condition is to be prevented. Examples of metastatic clear cell renal cell carcinoma treatment include, but are not limited to, active surveillance, observation, surgical intervention (such as partial or radical nephrectomy), thermal ablation (such as cryoablation or radiofrequency ablation), arterial embolization, radiation therapy, immunotherapy (such as IL-2 or Bevacizumab plus interferon alpha), targeted therapy (such as VEGF pathway and mTOR inhibitors), systemic therapy or a combination thereof.

“Approved Drugs” for metastatic renal cell carcinoma include but are not limited to AFINITOR (Everolimus), PROLEUKIN (Aldesleukin), AVASTIN (Bevacizumab), INLYTA (Axitinib), NEXAVAR (Sorafenib), VOTRIENT (Pazopanib), SUTENT (Sunitinib), TORISEL (Temsirolimus), or a combination thereof.

“416 additional clear cell renal cell carcinoma genes” as used herein refers to the following genes that can be prognostic of renal cell carcinoma and are listed with the hazard ratio, p-value and q-value [Gene (HR; p-value; q-value)]: CCNB1 (0.73; 1.00E-06; 5.90E-05); TOP2A (0.72; 1.00E-06; 5.90E-05); NPM3 (0.73; 1.00E-06; 6.00E-05); MCM2 (0.72; 1.00E-06; 6.00E-05); ANLN (0.74; 6.00E-06; 0.000221); KIF23 (0.74; 6.00E-06; 0.000221); FSCN1 (0.75; 1.30E-05; 0.000398); RELN (0.76; 2.10E-05; 0.000557); MKI67 (0.77; 2.30E-05; 0.000561); KIAA0101 (0.77; 2.80E-05; 0.000566); ASPM (0.76; 2.80E-05; 0.000566); MELK (0.75; 3.30E-05; 0.000606); BIRC5 (0.77; 5.30E-05; 0.00086); NME1 (0.79; 5.90E-05; 0.000886); KLK1 (0.79; 0.000199; 0.002682); TTK (0.8; 0.000251; 0.003043); NCAPG (0.79; 0.000289; 0.003307); PSAT1 (0.8; 3.00E-04; 0.003307); SLPI (0.79; 0.000392; 0.004127); L1CAM (0.81; 0.000519; 0.005026); SPP1 (0.8; 0.000572; 0.005325); VHL (0.81; 0.000632; 0.005667); POLR2B (0.81; 0.000666; 0.005734); ITGB1 (0.81; 0.000711; 0.005734); PRC1 (0.79; 0.000802; 0.006261); IGFBP2 (0.82; 0.000881; 0.006667); IGF2BP3 (0.81; 0.001046; 0.007325); C9orf71 (1.23; 0.001059; 0.007325); RFC4 (0.82; 0.001128; 0.007589); AR (1.21; 0.001428; 0.009263); SLC9A1 (0.82; 0.001454; 0.009263); TUBA4A (0.82; 0.001632; 0.010133); HSP90AA1 (0.82; 0.00177; 0.010714); SLC4A3 (0.82; 0.001863; 0.011003); EZH2 (0.82; 0.002009; 0.011584); CD276 (0.82; 0.002173; 0.012233); PSMB9 (0.82; 0.002252; 0.012391); PSME2 (0.83; 0.002514; 0.013528); AQP4 (1.21; 0.002813; 0.014805); AKAP7 (0.84; 0.003068; 0.015807); DGKB (1.19; 0.003168; 0.01598); CALR (0.83; 0.003454; 0.016613); EGFR (1.2; 0.003499; 0.016613); C4BPA (0.83; 0.003703; 0.017242); PKM2 (0.84; 0.00402; 0.018364); NRIP (1.19; 0.004622; 0.020725); HIF1A (0.84; 0.005563; 0.024491); NCAPG2 (0.84; 0.005683; 0.024571); BHMT (1.18; 0.006673; 0.028347); SLC2A1 (0.85; 0.007456; 0.031112); RPS6KB1 (0.85; 0.007581; 0.031112); FXYD1 (1.18; 0.007904; 0.031895); NPR3 (1.18; 0.008396; 0.032673); VEGFC (0.84; 0.0084; 0.032673); RELA (0.85; 0.008501; 0.032673); IL13RA2 (0.85; 0.010701; 0.040486); IL8 (0.86; 0.011146; 0.041518); KLK6 (0.86; 0.011996; 0.044009); FLT1 (1.16; 0.012525; 0.045264); CXCL1 (0.86; 0.013735; 0.048908); GALNTS (0.85; 0.014033; 0.049241); TNFRSF10B (0.86; 0.014263; 0.049336); PDCD2 (0.86; 0.014938; 0.050944); CXCL6 (0.86; 0.016077; 0.052848); CD44 (0.86; 0.016114; 0.052848); CD97 (0.86; 0.016152; 0.052848); TIMP2 (0.86; 0.016575; 0.053454); TAP1 (0.86; 0.016985; 0.053454); CYP2J2 (1.15; 0.016999; 0.053454); ATAD2 (0.86; 0.017438; 0.054132); ENO2 (0.86; 0.017671; 0.05416); CDC25A (0.86; 0.019002; 0.05751); SLC16A1 (0.87; 0.019821; 0.059251); PGF (1.16; 0.020496; 0.060033); TIMP1 (0.86; 0.020579; 0.060033); FRMD3 (1.15; 0.021449; 0.061639); EDNRB (1.15; 0.021639; 0.061639); RPL27A (0.87; 0.02239; 0.062885); BCL2 (1.14; 0.022595; 0.062885); INSR (1.14; 0.026044; 0.070682); MAFG (0.88; 0.026094; 0.070682); TYMS (0.87; 0.026273; 0.070682); HIF2A (1.14; 0.027701; 0.073704); AQP1 (1.14; 0.029085; 0.074381); TYMP (0.87; 0.029246; 0.074381); PAPPA (0.87; 0.029461; 0.074381); PDCD1 (0.87; 0.029605; 0.074381); CHL1 (0.87; 0.029679; 0.074381); APOLD1 (1.15; 0.029895; 0.074381); FLT4 (1.15; 0.030354; 0.074381); FOSL1 (0.86; 0.030413; 0.074381); GIPC2 (1.14; 0.032563; 0.078844); PSMB5 (0.87; 0.034602; 0.08295); EGLN3 (1.14; 0.034964; 0.082997); SGK1 (1.14; 0.036185; 0.085063); CD82 (0.88; 0.039478; 0.091605); GSK3B (0.88; 0.039725; 0.091605); ENPP2 (1.13; 0.040855; 0.09247); TRIM38 (0.88; 0.041239; 0.09247); ARL4D (0.88; 0.041273; 0.09247); LUM (0.88; 0.041971; 0.09247); VIM (0.87; 0.04201; 0.09247); EPAS1 (1.13; 0.042469; 0.092638); MAPT (1.13; 0.046074; 0.098031); MME (1.14; 0.046138; 0.098031); APLNR (1.13; 0.046156; 0.098031); NUDT6 (0.89; 0.046647; 0.098214); FAT1 (0.89; 0.047749; 0.099667); TAP2 (0.88; 0.048616; 0.100609); PLAU (0.88; 0.049382; 0.101329); FSCN3 (1.13; 0.050468; 0.101808); CARD8 (0.88; 0.050502; 0.101808); RACGAP1 (0.88; 0.051311; 0.101808); POSTN (0.88; 0.051545; 0.101808); RRAGB (0.88; 0.051718; 0.101808); ERAP1 (0.89; 0.052549; 0.102196); FGFR1 (0.88; 0.053132; 0.102196); FGFR4 (0.88; 0.053181; 0.102196); TGFBR3 (1.12; 0.05608; 0.106917); COL6A2 (0.88; 0.057621; 0.108997); MACC1 (1.12; 0.058356; 0.109532); CSPG4 (1.13; 0.058998; 0.109885); PDCD1LG2 (0.89; 0.061852; 0.114321); CTNNA3 (1.12; 0.062501; 0.114646); RGSS (1.12; 0.064041; 0.116586); CCNE2 (0.89; 0.064619; 0.116761); ANKRD36 (1.12; 0.065588; 0.117635); TMEM47 (1.12; 0.06858; 0.122097); TFPI2 (0.89; 0.069106; 0.122134); THBS2 (0.89; 0.071597; 0.124374); PIK3CA (0.89; 0.072558; 0.124374); PSME1 (0.89; 0.072943; 0.124374); GPC6 (1.11; 0.072985; 0.124374); ELTD1 (1.12; 0.073602; 0.124374); FAM134B (1.13; 0.073855; 0.124374); KDR (1.11; 0.074441; 0.124374); KRAS (0.9; 0.074863; 0.124374); IL6 (0.89; 0.07539; 0.124374); ICOSLG (0.9; 0.075718; 0.124374); MRPL22 (0.9; 0.076024; 0.124374); FGF2 (0.9; 0.077799; 0.126424); KLKP1 (0.89; 0.078959; 0.127453); TGFA (1.12; 0.07956; 0.127573); VDR (0.9; 0.080842; 0.128776); EMCN (1.11; 0.084304; 0.131904); ORM1 (0.89; 0.084434; 0.131904); ICOS (0.89; 0.08444; 0.131904); PLAUR (0.9; 0.088288; 0.137032); POU5F1 (1.11; 0.090257; 0.13875); NAPSA (1.11; 0.090542; 0.13875); SKP2 (0.9; 0.091502; 0.139123); PRKCD (0.9; 0.092332; 0.139123); EBAG9 (0.9; 0.092509; 0.139123); PRKG2 (1.11; 0.094433; 0.140642); CDC42 (0.9; 0.09468; 0.140642); CCND1 (1.1; 0.095784; 0.141327); ALDOA (0.9; 0.096309; 0.141327); PELO (0.9; 0.100354; 0.146376); ATP5G3 (0.91; 0.101746; 0.147518); ANXA2 (0.9; 0.104885; 0.15037); CSNK2A1P (1.11; 0.104955; 0.15037); CDK8 (0.91; 0.107778; 0.153506); NA (0.9; 0.108718; 0.153938); GAPDH (0.9; 0.111422; 0.15685); TSPAN7 (1.1; 0.114639; 0.160446); CITED4 (1.1; 0.116061; 0.161503); PSMB8 (0.91; 0.117564; 0.161751); FGD5 (1.1; 0.118104; 0.161751); ENG (1.1; 0.118952; 0.161751); RABEPK (0.91; 0.119161; 0.161751); PIGF (0.9; 0.119735; 0.161751); LMO2 (1.1; 0.120745; 0.161751); LDHA (0.91; 0.120915; 0.161751); CD34 (1.1; 0.124462; 0.164861); BAX (0.91; 0.124876; 0.164861); PTGS2 (0.91; 0.125283; 0.164861); PPAP2B (1.1; 0.127944; 0.167452); ECHS1 (0.91; 0.132248; 0.172155); SHC1 (0.91; 0.134057; 0.172966); CSNK2A1 (0.91; 0.1343; 0.172966); ESPL1 (0.91; 0.136192; 0.173624); KIF2A (0.91; 0.136331; 0.173624); BRCA2 (0.91; 0.137281; 0.173624); PDGFD (1.09; 0.137776; 0.173624); PECAM1 (1.09; 0.138396; 0.173624); CDK2 (0.91; 0.139899; 0.174604); PRKAA2 (1.09; 0.145484; 0.180644); A2M (1.09; 0.150989; 0.186523); FSCN2 (0.92; 0.151778; 0.186546); ANGPTL4 (1.09; 0.167059; 0.203944); CITED2 (1.09; 0.167618; 0.203944); BCL2L12 (0.92; 0.168553; 0.204056); PDGFRA (0.92; 0.173159; 0.208589); SMAD3 (0.92; 0.17445; 0.209104); AKT2 (0.92; 0.175479; 0.209301); CD53 (0.92; 0.176633; 0.209645); MAPK14 (0.92; 0.188747; 0.222931); PXK (0.92; 0.193087; 0.226754); IGFBP1 (0.93; 0.193857; 0.226754); PALMD (1.08; 0.196986; 0.228125); PIK3C2A (0.93; 0.197241; 0.228125); CCL5 (0.92; 0.197856; 0.228125); CTLA4 (0.92; 0.199168; 0.22855); CAV2 (1.09; 0.204297; 0.233329); MAP7 (1.08; 0.206349; 0.234566); NA (0.93; 0.208565; 0.235769); KHDRBS1 (0.92; 0.209355; 0.235769); GPR1 (0.93; 0.210434; 0.235888); TNNI3 (0.92; 0.216728; 0.241824); FHL1 (1.08; 0.222206; 0.246798); PIGR (0.93; 0.224232; 0.247474); FBLN1 (0.92; 0.224859; 0.247474); MTOR (0.93; 0.227933; 0.249505); ENTPD1 (1.08; 0.228765; 0.249505); IMP3 (0.93; 0.230092; 0.249685); PDIA3 (0.93; 0.230992; 0.249685); DCN (0.93; 0.236212; 0.254124); MTCH2 (0.93; 0.237647; 0.254124); MIF (0.93; 0.238248; 0.254124); PSMB10 (0.93; 0.240962; 0.255854); MYLIP (1.08; 0.241983; 0.255854); C10orf137 (0.93; 0.251787; 0.264546); IL13RA1 (0.93; 0.252389; 0.264546); TAPBP (0.93; 0.255062; 0.266195); FHIT (1.07; 0.264207; 0.274556); OSBPL1A (1.07; 0.275314; 0.284875); CDCP1 (0.93; 0.284522; 0.29315); PRR15L (0.94; 0.287664; 0.295133); RB1 (0.94; 0.289905; 0.296177); CXCL12 (0.94; 0.297889; 0.303054); TGFBR2 (0.94; 0.301461; 0.303358); ARG2 (1.06; 0.301806; 0.303358); PPP1R12B (1.06; 0.301946; 0.303358); CCL11 (0.94; 0.309658; 0.308967); RANBP1 (0.94; 0.311105; 0.308967); CTNNA1 (0.94; 0.311357; 0.308967); TYROBP (0.94; 0.312686; 0.30902); CNTN6 (0.94; 0.320839; 0.315788); PYCARD (1.06; 0.324827; 0.318418); ITGA7 (1.06; 0.331904; 0.323459); FZD1 (1.06; 0.332641; 0.323459); TIMP3 (1.06; 0.334665; 0.324086); SPRY1 (1.06; 0.335963; 0.324086); GUCY1B3 (0.94; 0.341689; 0.328302); CXCL9 (0.94; 0.344699; 0.329525); LIMCH1 (0.94; 0.346677; 0.329525); NOTCH3 (1.06; 0.347045; 0.329525); TMTC1 (0.95; 0.352511; 0.333408); ANGPT2 (1.06; 0.357337; 0.336657); GRIA1 (1.06; 0.358918; 0.336836); MMP9 (0.95; 0.364265; 0.339391); CD83 (1.06; 0.365433; 0.339391); CLDN18 (1.06; 0.365845; 0.339391); PTH1R (1.06; 0.367411; 0.339543); ABCA1 (1.06; 0.369973; 0.34061); IFNG (0.94; 0.372831; 0.341941); ICAM1 (1.06; 0.378138; 0.3455); C10orf54 (1.06; 0.383807; 0.347121); PTPRC (0.95; 0.383933; 0.347121); LDB2 (1.05; 0.387694; 0.347121); GPR116 (1.05; 0.388245; 0.347121); CDH3 (0.95; 0.388529; 0.347121); CXCR3 (0.95; 0.389588; 0.347121); MUC16 (0.94; 0.390663; 0.347121); SPARCL1 (1.05; 0.391382; 0.347121); HIF3A (0.95; 0.394115; 0.348059); CDH1 (1.06; 0.396206; 0.348059); MUC1 (0.95; 0.39776; 0.348059); CDKN1B (0.95; 0.399549; 0.348059); TNFRSF10D (1.05; 0.399627; 0.348059); TNFRSF6B (0.95; 0.406859; 0.351885); GSTT2 (1.05; 0.406926; 0.351885); VEGFB (0.95; 0.409087; 0.352495); CEACAM6 (1.05; 0.411993; 0.35374); GSN (1.05; 0.414425; 0.35457); TNFSF11 (1.05; 0.416565; 0.355131); FRS2 (0.95; 0.418021; 0.355131); DPYD (0.95; 0.419854; 0.355131); KRT7 (0.95; 0.422829; 0.355131); NA (1.05; 0.426054; 0.355131); SYNPO (1.05; 0.426054; 0.355131); BLMH (0.95; 0.426232; 0.355131); GenBank: AF131 (1.05; 0.426814; 0.355131); PDGFRB (1.05; 0.432358; 0.358512); ABCC1 (0.95; 0.435689; 0.359797); VTCN1 (0.95; 0.437896; 0.359797); PDF (0.96; 0.438606; 0.359797); ITGA4 (0.96; 0.441754; 0.359797); S100A8 (0.95; 0.441985; 0.359797); TSC2 (0.95; 0.442824; 0.359797); CD274 (0.95; 0.447528; 0.361732); MLL2 (0.95; 0.448194; 0.361732); CXCL11 (0.96; 0.459174; 0.369363); HIF1AN (0.96; 0.462105; 0.370351); NOS2 (0.96; 0.463821; 0.370351); BARD1 (0.96; 0.46499; 0.370351); TOX3 (0.96; 0.469142; 0.372433); MMP2 (0.95; 0.476896; 0.377351); ASB2 (0.96; 0.482144; 0.379174); TNFSF10 (0.96; 0.482332; 0.379174); TP53 (0.96; 0.490443; 0.384302); IGFBP7 (0.96; 0.496835; 0.388055); ITGA6 (1.04; 0.49887; 0.388392); LOX (1.04; 0.501506; 0.389193); SUSD5 (1.04; 0.503752; 0.389687); TNF (1.04; 0.505638; 0.3899); RBM33 (0.96; 0.509727; 0.391805); GAS6 (0.96; 0.515023; 0.394623); AP4B1 (0.96; 0.518196; 0.395802); ERAP2 (1.04; 0.522006; 0.397458); COL5A2 (0.96; 0.525943; 0.399153); MYOCD (1.04; 0.52753; 0.399153); KNG1 (0.96; 0.531918; 0.40122); MXRA5 (0.96; 0.534228; 0.401711); SYTL1 (0.96; 0.538472; 0.402517); MEM (0.96; 0.538624; 0.402517); EGLN1 (0.96; 0.542632; 0.404264); VEGFA (1.04; 0.560267; 0.415751); SFXN4 (0.97; 0.561484; 0.415751); CDKN2A (1.04; 0.576216; 0.423862); SNRK (1.03; 0.577509; 0.423862); EPCAM (1.03; 0.57769; 0.423862); LPIN3 (1.04; 0.581508; 0.424513); ARSD (1.04; 0.582084; 0.424513); ASPN (0.96; 0.594478; 0.43225); TRIP11 (0.97; 0.596388; 0.43234); BNIP3 (0.97; 0.598664; 0.432695); CTNNA2 (0.97; 0.605936; 0.433674) AK023558-ZNF468 (0.97; 0.606389; 0.433674); IGF1R (1.03; 0.606933; 0.433674); MDM2 (0.97; 0.607183; 0.433674); EPO (1.03; 0.612122; 0.435916); BSG (0.97; 0.614213; 0.436122); PRSS2 (0.97; 0.616944; 0.43678); CA9 (1.03; 0.620189; 0.437797); EGLN2 (1.03; 0.623853; 0.438948); NA (1.03; 0.626251; 0.438948); ICAM3 (0.97; 0.627257; 0.438948); CTSD (0.97; 0.629207; 0.439043); POU5F1B (1.03; 0.637651; 0.443656); MAPK1 (1.03; 0.650222; 0.451107); FOXO4 (0.97; 0.661982; 0.457953); SELE (1.03; 0.667249; 0.460282); GAS2L1 (1.03; 0.669773; 0.460711); TCF4 (1.03; 0.681883; 0.466053); SYNPO2L (0.97; 0.682019; 0.466053); NA (1.03; 0.684488; 0.466053); SAT2 (0.97; 0.68524; 0.466053); MMPI (1.03; 0.692435; 0.469628); TINAGL1 (1.02; 0.700484; 0.47376); APBB1IP (1.02; 0.704862; 0.475393); IFITM2 (1.02; 0.707283; 0.475701); GMNN (0.98; 0.716424; 0.480514); JUN (1.02; 0.724548; 0.484621); CXCL10 (0.98; 0.72753; 0.485049); DMBT1 (1.02; 0.730104; 0.485049); CRISPLD2 (0.98; 0.731234; 0.485049); TRIB2 (1.02; 0.733202; 0.485049); BIRC2 (1.02; 0.735259; 0.485085); DIABLO (0.98; 0.741935; 0.488159); GCH1 (1.02; 0.747311; 0.490363); CCL4 (1.02; 0.753742; 0.493246); TAF1C (0.98; 0.756512; 0.493725); CDH6 (0.98; 0.758673; 0.493804); CDH11 (0.98; 0.761092; 0.49405); TUBA4B (1.02; 0.787544; 0.509854); CTNNB1 (0.98; 0.792129; 0.511455); AGPAT9 (1.02; 0.797492; 0.513549); GUCY1A2 (0.98; 0.802881; 0.515648); RBP4 (1.02; 0.809857; 0.518752); FNIP1 (1.01; 0.814819; 0.520331); COL14A1 (0.99; 0.81662; 0.520331); PALM2 (0.99; 0.82541; 0.524551); SFTPB (1.01; 0.831159; 0.526822); RACGAP1P (1.01; 0.836079; 0.528557); PTEN (1.01; 0.848435; 0.532991); LRRC39 (0.99; 0.848498; 0.532991); MGP (0.99; 0.851067; 0.532991); FIGN (0.99; 0.85308; 0.532991); FYN (0.99; 0.854099; 0.532991); SERPINE1 (1.01; 0.858539; 0.533201); SPRY4 (1.01; 0.85884; 0.533201); IL12RB2 (0.99; 0.868484; 0.537809); COG8 (0.99; 0.871916; 0.538557); TRIP6 (1.01; 0.883124; 0.544092); PTENP1 (1.01; 0.885836; 0.544377); JUP (0.99; 0.889474; 0.544386); SAT1 (0.99; 0.890384; 0.544386); CCL2 (0.99; 0.893351; 0.544386); CLDN1 (1.01; 0.894843; 0.544386); PCDHAl (1.01; 0.901339; 0.546549); BCAP31 (0.99; 0.904695; 0.546549); ITGA1 (1.01; 0.905171; 0.546549); TP53AIP1 (0.99; 0.914407; 0.550209); PRDX2 (1.01; 0.915777; 0.550209); CD40 (0.99; 0.918784; 0.550649); IFIT1 (0.99; 0.930374; 0.556219); TNFAIP6 (1; 0.935841; 0.558109); SOSTDC1 (1; 0.94359; 0.559691); EMILIN3 (1; 0.947431; 0.559691); EP300 (1; 0.951605; 0.559691); PLIN2 (1; 0.954319; 0.559691); AXL (1; 0.955667; 0.559691); AKAP2 (1; 0.956347; 0.559691); BNC2 (1; 0.957194; 0.559691); NRP1 (1; 0.958909; 0.559691); KRASP1 (1; 0.959298; 0.559691); CRP (1; 0.970168; 0.563443); CLU (1; 0.974115; 0.563443); TPK1 (1; 0.979688; 0.563443); NOTCH1 (1; 0.979989; 0.563443); CAV1 (1; 0.980456; 0.563443); CA12 (1; 0.98059; 0.563443); VCAM1 (1; 0.982017; 0.563443); COQ6 (1; 0.995481; 0.569818); BCKDHB (1; 0.999435; 0.570731).

Approximately one third of patients newly diagnosed with RCC have metastatic disease, and after treatment for localized RCC, 25-50% of patients will suffer recurrence. The prognosis associated with recurrent and metastatic RCC is poor. However, the survival for individual patients can vary widely. Patients can be stratified into risk groups based on readily available clinical parameters such as performance status, serum lactate dehydrogenase, hemoglobin, serum calcium, and length of time between initial diagnosis and treatment. These Memorial Sloan Kettering Cancer Center (MSKCC) Adverse Clinical Risk Factors were used to stratify the randomization for the parent clinical trial of the study, CALGB90206.

However, there is a need for molecular biomarkers that can predict survival. This study developed a multi-marker prognostic signature from a phase III, randomized clinical trial in RCC in which eligibility is clearly defined and outcomes are rigorously recorded. Clear cell RCC, tumor tissue is routinely available from cytoreductive nephrectomy or diagnostic biopsy, therefore, we used formalin-fixed, paraffin-embedded tumors, which are routinely collected and stored in all pathology departments.

CALGB 90206 randomized patients with newly diagnosed clear cell RCC to Interferon (IFN) or IFN plus bevacizumab. The primary endpoint was overall survival (OS), and secondary endpoints were progression free survival and safety (PFS). The majority (85%) of patients underwent a cytoreductive nephrectomy, and 90% had favorable or intermediate prognosis based on number of MSKCC Adverse Clinical Risk Factors. At interim analysis, the median PFS was 5.2 months in the IFN group and 8.5 months in the IFN plus bevacizumab group (p<0.0001). However, there was no significant difference in OS. The OS was 17.4 mos in the IFN group and 18.3 months in the combination arm. Furthermore, subset analysis failed to identify any clinical variable associated with treatment response. CALGB 90206 demonstrated statistically longer progression-free survival (PFS) but no statistical overall survival (OS) benefit for patients treated with the combination therapy. Therefore, no clinical variable other than ACRF were included in our final model.

A similar international, randomized study (AVOREN) treated 649 patients in the front line setting with the same two treatments at the same doses. The PFS survivals were 10.2 and 5.4 months (p=0.0001). At interim analysis, the Supervisory Committee of Safety Data recommended administration of bevacizumab for patients in the placebo arm and regulatory agencies agreed to accept PFS for regulatory submission. When this information was made public, the CALGB Data Safety Monitoring Board made the independent decisions to also release the PFS data at an interim analysis. Neither CALGB90206 nor AVOREN demonstrated a difference in OS in the two study arms. This is likely due to cross overs and the majority of patients receiving one or more active therapies on disease progression before death since multiple VEGF-targeted therapies became available during the course of the trial.

The qPCR assay is well-established and robust, and routinely used in commercial laboratories. Our study used qPCR for targeted measurement of candidate biomarkers. It is well established that qPCR has a large dynamic range and is well-suited for measuring gene expressions using highly fragmented RNA found in archival tumor blocks stored at room temperature. Our assays started with tumor sections cut onto glass slides. The reference genes and the number of reference genes used to normalize gene expressions were empirically selected.

The genetic heterogeneity of RCC is well documented. To generate a signature that was less sensitive to sampling artifacts produced by tumor heterogeneity, we performed a separate analysis using untreated primary tumors from metastatic clear cell RCC patients that were sampled in two different areas. Genes with heterogeneous expression within individual patients were excluded from consideration in our multi-marker models.

We report a gene expression-based prognostic signature developed using primary untreated ccRCC collected as part of CALGB90206, which served as the registration trial for FDA approval of bevacizumab in combination with interferon alpha (INF). This is the first report of a molecular signature developed from a multicenter, phase III clinical trial of RCC. Results of multicenter studies are more convincing because tissues are less susceptible to systemic bias resulting from institution-specific tissue-handling protocols and are more likely to be representative. The parent trial clearly defines the patient cohort for which the signature can be applied. Furthermore, patient treatment and follow-up have been rigorously recorded, with oversight from a highly developed coordinating center.

The present invention is based, at least in part, on these findings. The present invention addresses the need in the art for methods of determining overall survival in a patient with metastatic renal cell carcinoma, and for guiding treatment options for the patients. This invention provides, among other things, a prognostic model for overall survival for patients with metastatic renal cell carcinoma, which is useful, inter alia, for guiding treatment for patients.

In this invention, we provide methods determining overall survival, by detecting CRYL1, CEP55, PCNA, TRAF2, HGF, CDK1, HSD17B10, USP6NL, ACTB, RPL13A, GUS, RPLP0, HPRT1 and SDHA gene expression levels. We also provide a method of selecting therapy and/or treatment, and selecting a clinical trial for patients with metastatic renal cell carcinoma. The invention further provides a process for patient risk stratification.

### Determination of Overall Survival

Various embodiments of the present invention provide for a method of determining overall survival in a subject, with metastatic clear cell renal cell carcinoma, comprising: obtaining a biological sample from a subject with metastatic clear cell renal cell carcinoma; assaying the biological sample to determine an expression level for a metastatic clear cell renal cell carcinoma gene and a reference gene, wherein the metastatic clear cell renal cell carcinoma (RCC) gene is selected from the group consisting of CRYL1, CEP55, PCNA, TRAF2, HGF, CDK1, HSD17B10, USP6NL and combinations thereof, wherein the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a coefficient or using a calculated coefficient, for each metastatic clear cell renal cell carcinoma gene; and determining that the subject has a good overall survival if the coefficient is negative with a low gene expression level or if the coefficient is positive with a high gene expression level and determining that the subject has poor overall survival if the coefficient is negative with a high gene expression level or if the coefficient is positive with a low gene expression level.

In various embodiments, the subject has a good overall survival if the coefficient is negative with a low normalized gene expression level or if the coefficient is positive with a high normalized gene expression level and determining that the subject has poor overall survival if the coefficient is negative with a high normalized gene expression level or if the coefficient is positive with a low normalized gene expression level.

In various embodiments, the metastatic clear cell RCC gene is a combination of two, three, four, five, six, seven or eight of CRYL1, CEP55, PCNA, TRAF2, HGF, CDK1, HSD17B10, and USP6NL.

In various embodiments, the reference gene is a combination of one, two, three, four, five or six of ACTB, RPL13A, GUS, RPLP0, HPRT1, and SDHA.

In various other embodiments, the method further comprises using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in determining overall survival.

In other embodiments, the method further comprises assaying the biological sample to determine an expression level for one or more of the 416 additional clear cell renal cell carcinoma genes.

In other embodiments, the method further comprises assaying the biological sample to determine an expression level of one or more of the 416 additional clear cell renal cell carcinoma genes; normalizing the one or more of the 416 additional clear cell renal cell carcinoma gene expression level to the reference gene expression level; calculating a coefficient or using a calculated coefficient for each of the one or more of the 416 additional clear cell renal cell carcinoma genes; and calculating a risk score from the coefficients.

Various embodiments of the present invention also provide for a method of determining overall survival in a subject, with metastatic clear cell renal cell carcinoma, comprising: obtaining a biological sample from a subject with metastatic clear cell renal cell carcinoma; assaying the biological sample to determine an expression level for a metastatic clear cell renal cell carcinoma gene, wherein the metastatic clear cell renal cell carcinoma gene is selected from the group consisting of CRYL1, CEP55, PCNA, TRAF2, HGF, CDK1, HSD17B10, USP6NL and combinations thereof; and determining that the subject has a good overall survival if there is an increased expression level of CRYL1, PCNA, CDK1, or a combination thereof, or if there is an decreased expression level of TRAF2, USP6NL, CEP55, HGF, HSD17B10 or a combination thereof, and determining that the subject has poor overall survival if there is an decrease expression level of CRYL1, PCNA, CDK1, or a combination thereof, or if there is an increase expression level of TRAF2, USP6NL, CEP55, HGF, HSD17B10 or a combination thereof.

In various embodiments, the method further comprises assaying the biological sample to determine an expression level for a reference gene, wherein the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; and determining that the subject has a good overall survival if there is an increased expression level of CRYL1, PCNA, CDK1, or a combination thereof, or if there is an decreased expression level of TRAF2, USP6NL, CEP55, HGF, HSD17B10 or a combination thereof, and determining that the subject has poor overall survival if there is an decrease expression level of CRYL1, PCNA, CDK1, or a combination thereof, or if there is an increase expression level of TRAF2, USP6NL, CEP55, HGF, HSD17B10 or a combination thereof.

In other embodiments, a decreased expression level of CRYL1, PCNA, CDK1 or a combination thereof, is indicative of a poor overall survival for the patient. In yet other embodiments, an increased expression level of TRAF2, USP6NL, CEP55, HGF, HSD17B10 or a combination thereof, is indicative of poor overall survival for the patient.

In various other embodiments, an increased expression level of CRYL1, PCNA, CDK1 or a combination thereof, is indicative of good overall survival for the patient. In yet other embodiments, a decreased expression level of TRAF2, USP6NL, CEP55, HGF, HSD17B10 or a combination thereof, is indicative of good overall survival for the patient.

In various embodiments, the metastatic clear cell RCC gene is a combination of two, three, four, five, six, seven or eight of CRYL1, CEP55, PCNA, TRAF2, HGF, CDK1, HSD17B10, and USP6NL.

In various embodiments, the reference gene is a combination of one, two, three, four, five or six of ACTB, RPL13A, GUS, RPLP0, HPRT1, and SDHA.

In various other embodiments, the method further comprises using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in determining overall survival.

In yet other embodiments, the method further comprises assaying the biological sample to determine an expression level for one or more of the 416 additional clear cell renal cell carcinoma genes.

In other embodiments, the method further comprises assaying the biological sample to determine an expression level of one or more of the 416 additional clear cell renal cell carcinoma genes; normalizing the one or more of the 416 additional clear cell renal cell carcinoma gene expression level to the reference gene expression level; calculating a coefficient or using a calculated coefficient for each of the one or more of the 416 additional clear cell renal cell carcinoma genes; and calculating a risk score from the coefficients.

Various embodiments of the present invention also provide for a method of determining overall survival in a subject with metastatic clear cell renal cell carcinoma, comprising: obtaining a biological sample from the subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a coefficient or using a calculated coefficient for the metastatic clear cell renal cell carcinoma gene; calculating a risk score from the coefficient; and identifying the subjects with low risk scores as having better overall survival than subjects with high risk scores.

In various embodiments, identifying the subjects comprises identifying whether the subject is in a low, intermediate or high risk group for metastatic clear cell renal cell carcinoma, wherein a subject with a low risk score has a good overall survival, a subject with an intermediate risk score has an intermediate overall survival and the subject with a high risk score has a poor overall survival.

In various embodiments, the metastatic clear cell RCC gene is a combination of three, four, five, six, seven or eight of CRYL1, CEP55, PCNA, TRAF2, HGF, CDK1, HSD17B10, and USP6NL.

In various embodiments, the reference gene is a combination of two, three, four, five or six of ACTB, RPL13A, GUS, RPLP0, HPRT1, and SDHA.

In various other embodiments, the method further comprises using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in determining overall survival. The method further comprises calculating a risk score based on the one or more MSKCC adverse clinical risk factors, and using the risk score in conjunction with the risk score calculated from the one, two, three, four, five, six, seven or eight metastatic clear cell RCC gene. Calculating a risk score based on the one or more MSKCC adverse clinical risk factors can be done as described herein.

In yet other embodiments, the method further comprises assaying the biological sample to determine an expression level for one or more of the 416 additional clear cell renal cell carcinoma genes.

In other embodiments, the method further comprises assaying the biological sample to determine an expression level of one or more of the 416 additional clear cell renal cell carcinoma genes; normalizing the one or more of the 416 additional clear cell renal cell carcinoma gene expression level to the reference gene expression level; calculating a coefficient or using a calculated coefficient for each of the one or more of the 416 additional clear cell renal cell carcinoma genes; and calculating a risk score from the coefficients.

Various embodiments of the present invention also provide for a method of determining overall survival in a subject with metastatic clear cell renal cell carcinoma, comprising: obtaining a biological sample from the subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a hazard ratio or using a calculated hazard ratio for the metastatic clear cell renal cell carcinoma gene; calculating a risk score from the hazard ratio; and stratifying the subject into risk groups based on the risk score, wherein subjects with low risk scores have better overall survival than subjects with high risk scores.

In various embodiments, stratifying the subject into risk groups comprises stratifying the subject into a low, intermediate and high risk group for metastatic clear cell renal cell carcinoma from the risk score, wherein a subject in a low risk group has a good overall survival, a subject in an intermediate risk group has an intermediate overall survival and the subject in a high risk group has a poor overall survival.

In various embodiments, the metastatic clear cell RCC gene is a combination of two, three, four, five, six, seven or eight of CRYL1, CEP55, PCNA, TRAF2, HGF, CDK1, HSD17B10, and USP6NL.

In various embodiments, the reference gene is a combination of two, three, four, five or six of ACTB, RPL13A, GUS, RPLP0, HPRT1, and SDHA.

In various other embodiments, the method further comprises using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in determining overall survival. The method further comprises calculating a risk score based on the one or more MSKCC adverse clinical risk factors, and using the risk score in conjunction with the risk score calculated from the one, two, three, four, five, six, seven or eight metastatic clear cell RCC gene. Calculating a risk score based on the one or more MSKCC adverse clinical risk factors can be done as described herein.

In yet other embodiments, the method further comprises assaying the biological sample to determine an expression level for one or more of the 416 additional clear cell renal cell carcinoma genes.

In other embodiments, the method further comprises assaying the biological sample to determine an expression level of one or more of the 416 additional clear cell renal cell carcinoma genes; normalizing the one or more of the 416 additional clear cell renal cell carcinoma gene expression level to the reference gene expression level; calculating a coefficient or using a calculated coefficient for each of the one or more of the 416 additional clear cell renal cell carcinoma genes; and calculating a risk score from the coefficients.

### Patient Risk Stratification

Various embodiments of the present invention provide for a process of patient risk stratification, comprising: obtaining a biological sample from a subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, wherein the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a coefficient or using a calculated coefficient for each metastatic clear cell renal cell carcinoma gene; calculating a risk score from the coefficients; and stratifying the subject into risk groups based on the risk score, wherein subjects with low risk scores have better overall survival than subjects with high risk scores.

In various embodiments, stratifying the subject into risk groups comprises stratifying the subject into a low, intermediate and high risk group for metastatic clear cell renal cell carcinoma from the risk score, wherein a subject in a low risk group has a good overall survival, a subject in an intermediate risk group has an intermediate overall survival and the subject in a high risk group has a poor overall survival.

In various embodiments, the metastatic clear cell RCC gene is a combination of two, three, four, five, six, seven or eight of CRYL1, CEP55, PCNA, TRAF2, HGF, CDK1, HSD17B10, and USP6NL.

In various embodiments, the reference gene is a combination of two, three, four, five or six of ACTB, RPL13A, GUS, RPLP0, HPRT1, and SDHA.

In various other embodiments, the method further comprises using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in stratifying the subject into the risk groups. The method further comprises calculating a risk score based on the one or more MSKCC adverse clinical risk factors, and using the risk score in conjunction with the risk score calculated from the one, two, three, four, five, six, seven or eight metastatic clear cell RCC gene. Calculating a risk score based on the one or more MSKCC adverse clinical risk factors can be done as described herein.

In yet other embodiments, the method further comprises assaying the biological sample to determine an expression level for one or more of the 416 additional clear cell renal cell carcinoma genes.

In other embodiments, the method further comprises assaying the biological sample to determine an expression level of one or more of the 416 additional clear cell renal cell carcinoma genes; normalizing the one or more of the 416 additional clear cell renal cell carcinoma gene expression level to the reference gene expression level; calculating a coefficient or using a calculated coefficient for each of the one or more of the 416 additional clear cell renal cell carcinoma genes; and calculating a risk score from the coefficients.

Various embodiments of the present invention provide for a process of patient risk stratification, comprising: obtaining a biological sample from the subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a hazard ratio or using a calculated hazard ratio for each metastatic clear cell renal cell carcinoma gene; calculating a risk score from the hazard ratio; and stratifying the subject into risk groups based on the risk score, wherein subjects with low risk scores have better overall survival than subjects with high risk scores.

In various embodiments, stratifying the subject into risk groups comprises stratifying the subject into a low, intermediate and high risk group for metastatic clear cell renal cell carcinoma from the risk score, wherein a subject in a low risk group has a good overall survival, a subject in an intermediate risk group has an intermediate overall survival and the subject in a high risk group has a poor overall survival.

In various embodiments, the metastatic clear cell RCC gene is a combination of two, three, four, five, six, seven or eight of CRYL1, CEP55, PCNA, TRAF2, HGF, CDK1, HSD17B10, and USP6NL.

In various embodiments, the reference gene is a combination of one, two, three, four, five or six of ACTB, RPL13A, GUS, RPLP0, HPRT1, and SDHA.

In various other embodiments, the method further comprises using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in stratifying the subject into the risk groups. The method further comprises calculating a risk score based on the one or more MSKCC adverse clinical risk factors, and using the risk score in conjunction with the risk score calculated from the one, two, three, four, five, six, seven or eight metastatic clear cell RCC gene. Calculating a risk score based on the one or more MSKCC adverse clinical risk factors can be done as described herein.

In yet other embodiments, the method further comprises assaying the biological sample to determine an expression level for one or more of the 416 additional clear cell renal cell carcinoma genes.

In other embodiments, the method further comprises assaying the biological sample to determine an expression level of one or more of the 416 additional clear cell renal cell carcinoma genes; normalizing the one or more of the 416 additional clear cell renal cell carcinoma gene expression level to the reference gene expression level; calculating a coefficient or using a calculated coefficient for each of the one or more of the 416 additional clear cell renal cell carcinoma genes; and calculating a risk score from the coefficients.

Selecting Therapy and/or Treatment

Various embodiments of the present invention provide for a method of selecting a therapy and/or treatment for metastatic renal cell carcinoma, comprising: obtaining a biological sample from a subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, wherein the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a coefficient or using a calculated coefficient for each metastatic clear cell renal cell carcinoma gene; calculating a risk score from the coefficients; stratifying the subject into risk groups based on the risk score, wherein subjects with low risk scores have better overall survival than subjects with high risk scores; and selecting a first therapy, a second therapy, and/or a third therapy based on the subject's risk score.

In various embodiments, stratifying the subject into risk groups comprises stratifying the subject into a low, intermediate and high risk group for metastatic clear cell renal cell carcinoma from the risk score, wherein a subject in a low risk group has a good overall survival, a subject in an intermediate risk group has an intermediate overall survival and the subject in a high risk group has a poor overall survival; and a first therapy, a second therapy, and/or a third therapy comprises selecting a first therapy for a subject in the low, intermediate and high risk group, selecting a second therapy for a subject in the low or intermediate risk group and selecting a third therapy, or a combination of the first, second and third therapy for a subject in a high risk group.

In various embodiments, the metastatic clear cell RCC gene is a combination of two, three, four, five, six, seven or eight of CRYL1, CEP55, PCNA, TRAF2, HGF, CDK1, HSD17B10, and USP6NL.

In various embodiments, the reference gene is a combination of one, two, three, four, five or six of ACTB, RPL13A, GUS, RPLP0, HPRT1, and SDHA.

In various other embodiments, the method further comprises using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in selecting a therapy for the subject. The method further comprises calculating a risk score based on the one or more MSKCC adverse clinical risk factors, and using the risk score in conjunction with the risk score calculated from the one, two, three, four, five, six, seven or eight metastatic clear cell RCC gene. Calculating a risk score based on the one or more MSKCC adverse clinical risk factors can be done as described herein.

In yet other embodiments, the method further comprises assaying the biological sample to determine an expression level for one or more of the 416 additional clear cell renal cell carcinoma genes.

In other embodiments, the method further comprises assaying the biological sample to determine an expression level of one or more of the 416 additional clear cell renal cell carcinoma genes; normalizing the one or more of the 416 additional clear cell renal cell carcinoma gene expression level to the reference gene expression level; calculating a coefficient or using a calculated coefficient for each of the one or more of the 416 additional clear cell renal cell carcinoma genes; and calculating a risk score from the coefficients.

Various embodiments of the present invention provide for a method of selecting a therapy and/or treatment for metastatic renal cell carcinoma, comprising: obtaining a biological sample from a subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, wherein the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a hazard ratio or using a hazard ratio for each metastatic clear cell renal cell carcinoma gene; calculating a risk score from the hazard ratio; stratifying the subject into risk groups based on the risk score, wherein subjects with low risk scores have better overall survival than subjects with high risk scores; and selecting a first therapy, a second therapy, and/or a third therapy based on the subject's risk score.

In various embodiments, stratifying the subject into risk groups comprises stratifying the subject into a low, intermediate and high risk group for metastatic clear cell renal cell carcinoma from the risk score, wherein a subject in a low risk group has a good overall survival, a subject in an intermediate risk group has an intermediate overall survival and the subject in a high risk group has a poor overall survival; and a first therapy, a second therapy, and/or a third therapy comprises selecting a first therapy for a subject in the low, intermediate and high risk group, selecting a second therapy for a subject in the low or intermediate risk group and selecting a third therapy, or a combination of the first, second and third therapy for a subject in a high risk group.

In various embodiments, the metastatic clear cell RCC gene is a combination of two, three, four, five, six, seven or eight of CRYL1, CEP55, PCNA, TRAF2, HGF, CDK1, HSD17B10, and USP6NL.

In various embodiments, the reference gene is a combination of one, two, three, four, five or six of ACTB, RPL13A, GUS, RPLP0, HPRT1, and SDHA.

In various other embodiments, the method further comprises using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in selecting a therapy for the subject. The method further comprises calculating a risk score based on the one or more MSKCC adverse clinical risk factors, and using the risk score in conjunction with the risk score calculated from the one, two, three, four, five, six, seven or eight metastatic clear cell RCC gene. Calculating a risk score based on the one or more MSKCC adverse clinical risk factors can be done as described herein.

In yet other embodiments, the method further comprises assaying the biological sample to determine an expression level for one or more of the 416 additional clear cell renal cell carcinoma genes.

In other embodiments, the method further comprises assaying the biological sample to determine an expression level of one or more of the 416 additional clear cell renal cell carcinoma genes; normalizing the one or more of the 416 additional clear cell renal cell carcinoma gene expression level to the reference gene expression level; calculating a coefficient or using a calculated coefficient for each of the one or more of the 416 additional clear cell renal cell carcinoma genes; and calculating a risk score from the coefficients.

In various embodiments, patient counseling is given to a subject that has been stratified into a low, intermediate or high risk group.

In various embodiments, the first therapy is selected from the group consisting of surgical resection, radical or partial nephrectomy, active surveillance, palliative radiation therapy, metastasectomy and/or bisphonates. In various embodiments, the second therapy is a targeted therapy drug or immunotherapy. In various embodiments, the targeted therapy drug is selected from the group consisting of VEGF inhibitors or mTOR inhibitors. In various embodiments, the VEGF inhibitors are selected from the group consisting of Sunitinib, Pazopanib, Bevacizumab, Sorafenib, Axitinib, and combinations thereof. In various embodiments, the mTOR inhibitors are selected from the group consisting of Temsirolimus, Everolimus, and combinations thereof. In various embodiments, the immunotherapy is selected from the group consisting of high-dose Interleukin-2, low-dose Interleukin-2, Interferon-alpha 2a, Bevacizumab and Interferon-alpha. In various embodiments, the third therapy is thermal ablation, a combination of the first and second therapy, and combinations thereof. In various embodiments, thermal ablation comprises cryoablation and radiofrequency ablation (Table 1). “First therapy”, “second therapy” and “third therapy” do not mean that the therapies will be tried consecutively; it is just a convenient way to differentiate the three classes of therapies.

TABLE 1
Therapy Options for Low, Intermediate and High Risk Groups

Surgical resection
Nephrectomy (radical or partial)
Active surveillance
Supportive Care (i.e., Palliative radiation therapy,
Metastasectomy and Bisphosphonates)
Targeted Therapy:
VEGF Inhibitors:
Sunitinib
Pazopanib
Bevacizumab
Sorafenib
Axitinib
mTOR inhibitors:
Temsirolimus
Everolimus
Immunotherapy:
Low-dose interleukin 2 (IL-2)
High-dose interleukin 2 (IL-2)
Interferon alpha 2a (IFN-a)

In various embodiments, the invention further provides for administering the selected treatment and/or therapy.

### Clinical Trial Selection

Various embodiments of the present invention provide for a method of selecting a metastatic clear cell renal cell carcinoma subject for a clinical trial, comprising: obtaining a biological sample from a subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, wherein the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a coefficient or using a calculated coefficient for each metastatic clear cell renal cell carcinoma gene; calculating a risk score from the coefficients; stratifying the subject into risk groups based on the risk score, wherein subjects with low risk scores have better overall survival than subjects with high risk scores; and selecting the subject for a clinical trial if the subject falls within the appropriate risk group for the clinical trial.

In various embodiments, stratifying the subject into risk groups comprises stratifying the subject into a low, intermediate and high risk group for metastatic clear cell renal cell carcinoma from the risk score, wherein a subject in a low risk group has a good overall survival, a subject in an intermediate risk group has an intermediate overall survival and the subject in a high risk group has a poor overall survival; and wherein a subject in a low risk group is selected for a low risk group clinical trial, a subject in an intermediate risk group is selected for an intermediate risk group clinical trial and a subject in a high risk group is selected for a high risk group clinical trial.

In various other embodiments, the method further comprises assaying the biological sample to determine an expression level for one or more of the 416 additional clear cell renal cell carcinoma genes; normalizing the one or more of the 416 additional clear cell renal cell carcinoma genes expression level to the reference gene expression level; calculating a coefficient or using a calculated coefficient for each of the of one or more of the 416 additional clear cell renal cell carcinoma genes; and calculating a risk score from the coefficients.

In various embodiments, the metastatic clear cell RCC gene is a combination of two, three, four, five, six, seven or eight of CRYL1, CEP55, PCNA, TRAF2, HGF, CDK1, HSD17B10, and USP6NL.

In various embodiments, the reference gene is a combination of one, two, three, four, five or six of ACTB, RPL13A, GUS, RPLP0, HPRT1, and SDHA.

In various other embodiments, the method further comprises using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in selecting a subject for a clinical trial. The method further comprises calculating a risk score based on the one or more MSKCC adverse clinical risk factors, and using the risk score in conjunction with the risk score calculated from the one, two, three, four, five, six, seven or eight metastatic clear cell RCC gene. Calculating a risk score based on the one or more MSKCC adverse clinical risk factors can be done as described herein.

In yet other embodiments, the method further comprises assaying the biological sample to determine an expression level for one or more of the 416 additional clear cell renal cell carcinoma genes.

In other embodiments, the method further comprises assaying the biological sample to determine an expression level of one or more of the 416 additional clear cell renal cell carcinoma genes; normalizing the one or more of the 416 additional clear cell renal cell carcinoma gene expression level to the reference gene expression level; calculating a coefficient or using a calculated coefficient for each of the one or more of the 416 additional clear cell renal cell carcinoma genes; and calculating a risk score from the coefficients.

Various embodiments of the present invention provide for a method of selecting a metastatic clear cell renal cell carcinoma subject for a clinical trial, comprising: obtaining a biological sample from a subject with metastatic clear cell renal cell carcinoma; assaying the biological sample to determine an expression level for a metastatic clear cell renal cell carcinoma gene and a reference gene, wherein the metastatic clear cell renal cell carcinoma gene is selected from the group consisting of CRYL1, CEP55, PCNA, TRAF2, HGF, CDK1, HSD17B10, USP6NL and combinations thereof, wherein the reference gene is selected from the group consisting of ACTB, RPL13A, GUS, RPLP0, HPRT1, SDHA and combinations thereof; normalizing the metastatic clear cell renal cell carcinoma gene expression level to the reference gene expression level; calculating a hazard ratio or using a hazard ratio for each metastatic clear cell renal cell carcinoma gene; calculating a risk score from the hazard ratio; stratifying the subject into risk groups based on the risk score, wherein subjects with low risk scores have better overall survival than subjects with high risk scores; and selecting the subject for a clinical trial if the subject falls within the appropriate risk group for the clinical trial.

In various embodiments, stratifying the subject into risk groups comprises stratifying the subject into a low, intermediate and high risk group for metastatic clear cell renal cell carcinoma from the risk score, wherein a subject in a low risk group has a good overall survival, a subject in an intermediate risk group has an intermediate overall survival and the subject in a high risk group has a poor overall survival; and wherein a subject in a low risk group is selected for a low risk group clinical trial, a subject in an intermediate risk group is selected for an intermediate risk group clinical trial and a subject in a high risk group is selected for a high risk group clinical trial.

In various other embodiments, the method further comprises assaying the biological sample to determine an expression level for one or more of the 416 additional clear cell renal cell carcinoma genes; normalizing the one or more of the 416 additional clear cell renal cell carcinoma genes expression level to the reference gene expression level; calculating a coefficient or using a calculated coefficient for each of the of one or more of the 416 additional clear cell renal cell carcinoma genes; and calculating a hazard ratio from the coefficients.

In various embodiments, the metastatic clear cell RCC gene is a combination of two, three, four, five, six, seven or eight of CRYL1, CEP55, PCNA, TRAF2, HGF, CDK1, HSD17B10, and USP6NL.

In various embodiments, the reference gene is a combination of one, two, three, four, five or six of ACTB, RPL13A, GUS, RPLP0, HPRT1, and SDHA.

In various other embodiments, the method further comprises using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in selecting a subject for a clinical trial. The method further comprises calculating a risk score based on the one or more MSKCC adverse clinical risk factors, and using the risk score in conjunction with the risk score calculated from the one, two, three, four, five, six, seven or eight metastatic clear cell RCC gene. Calculating a risk score based on the one or more MSKCC adverse clinical risk factors can be done as described herein.

In yet other embodiments, the method further comprises assaying the biological sample to determine an expression level for one or more of the 416 additional clear cell renal cell carcinoma genes.

In other embodiments, the method further comprises assaying the biological sample to determine an expression level of one or more of the 416 additional clear cell renal cell carcinoma genes; normalizing the one or more of the 416 additional clear cell renal cell carcinoma gene expression level to the reference gene expression level; calculating a coefficient or using a calculated coefficient for each of the one or more of the 416 additional clear cell renal cell carcinoma genes; and calculating a risk score from the coefficients.

### Coefficients

In various embodiments, the metastatic clear cell renal cell carcinoma gene coefficients in these methods are: CDK1 is 0.089, CEP55 is −0.258, CRYL1 is 0.356, HGF is −0.086, HSD17B10 is −0.232, PCNA is 0.155, TRAF2 is −0.215 and USP6NL is −0.090.

In certain embodiments, the coefficient for the metastatic clear cell renal cell carcinoma gene is +/−5%, 10% or 15% of the coefficient stated above.

In various other embodiments, the coefficient falls within the 95% CI for the gene; for example, the range for CDK1 may be −0.139 to 0.316, for CEP55 maybe −0.456 to −0.062, CRYL1 maybe 0.172 to 0.540, HGF maybe −0.273 to 0.102, HSD17B10 maybe −0.434 to −0.029, PCNA maybe −0.073 to 0.381, TRAF2 maybe −0.374 to −0.057 and USP6NL maybe −0.286 to 0.105.

In various embodiments, a coefficient is calculated for each MSKCC adverse clinical risk factor treated as a categorical variable and 0 MSKCC adverse clinical risk factors serving as the reference. In various embodiments, the MSKCC adverse clinical risk factor coefficient for 1 and/or 2 MSKCC adverse clinical risk factors is 0.276. In various embodiments, the MSKCC adverse clinical risk factor coefficient for 3 or more MSKCC adverse clinical risk factors is 0.954.

In various other embodiments, the 95% CI falls within a range, for example the range for 1 and/or 2 MSKCC adverse clinical risk factors is −0.063 to 0.615. In yet other embodiments, the 95% CI falls within a range, for example the range for 3 or more MSKCC adverse clinical risk factors is 0.383 to 1.523.

In various embodiments, the coefficient is calculated from the slope of a multi-variant regression model. In some embodiments, the coefficient is the slope of the multi-variant regression model. In other embodiments, the coefficients come from a model that includes the eight genes and the MSKCC adverse clinical risk factors. In various other embodiments, the coefficients are calculated from a model that includes the MSKCC adverse clinical risk factors or the eight genes.

### Hazard Ratio

In various embodiments, a hazard ratio is calculated. In certain embodiments, the hazard ratio is calculated using the equation HR=exp(coefficient).

In various embodiments, the calculated hazard ratio in these methods for the metastatic clear cell renal cell carcinoma gene CDK1 is 1.093, CEP55 is 0.772, CRYL1 is 1.428, HGF is 0.918, HSD17B10 is 0.793, PCNA is 1.167, TRAF2 is 0.806 and USP6NL is 0.914.

In other embodiments, the calculated hazard ratio (HR) falls within the 95% confidence interval (CI), and thus, upper and lower limits for the HR is: CDK1 is 0.870 and 1.372, CEP55 is 0.634 and 0.940, CRYL1 is 1.188 and 1.716, HGF is 0.761 and 1.107, HSD17B10 is 0.648 and 0.971, PCNA is 0.930 and 1.464, TRAF2 is 0.688 and 0.945 and USP6NL is 0.751 and 1.111, respectively.

In various other embodiments, the calculated HR for 1 and/or 2 MSKCC adverse clinical risk factors is 1.317. In yet other embodiments, the calculated HR for 3 or more MSKCC adverse clinical risk factors is 2.596. In some embodiments, the 95% confidence interval (CI), upper and lower limits for 1 and/or 2 MSKCC adverse clinical risk factors is 0.939 and 1.849, respectively. In some other embodiments, the 95% confidence interval (CI), upper and lower limits for 3 or more MSKCC adverse clinical risk factors is 1.467 and 4.594, respectively.

### Risk Scores and Risk Groups

In various embodiments, a risk score is calculated for each patient from the regression coefficients.

In various embodiments, each gene's risk score equals the gene's coefficient multiplied by the normalized gene expression. The risk score for each gene and/or risk factor is added together with the calculated risk score for the other genes and/or risk factors of interest to obtain an overall risk score. The overall risk score is used herein in accordance with various embodiments of the invention.

In various embodiments, the normalized gene expression is the normalized Delta Ct expression, e.g., ΔΔC_{T}. Thus, the higher value, the lower expression of the gene.

In various embodiments, the risk score (RS) is calculated using the equation RS=Σ(ln(HR)×normalized gene expression), which is the sum of the prognostic genes analyzed.

Thus, for example, in embodiments wherein eight metastatic clear cell renal cell carcinoma genes are assessed, and MSKCC Adverse Clinical Risk Factors are assessed, the risk score used in accordance with various embodiments of the present invention (e.g., to determine overall survival, to stratify a patient's risk, select therapies or treatments, or select clinical trials) is the sum of each gene's calculated risk score and the risk score determined from MSKCC Adverse Clinical Risk Factors.

In various embodiments, the risk score is calculated from the hazard ratio. For example, the hazard ratio of one, two, three, four, five, six, seven or eight of the metastatic clear cell renal cell carcinoma genes are added together. In another example, the hazard ratio of one, two, three, four, five, six, seven or eight of the metastatic clear cell renal cell carcinoma genes (CRYL1, CEP55, PCNA, TRAF2, HGF, CDK1, HSD17B10, and USP6NL), and the hazard ratio determined from the MSKCC Adverse Clinical Risk Factors are added together.

In various embodiments, the risk score is then calibrated for survival.

In various embodiments, the risk scores are used to stratify subjects into various prognostic groups. For example, in some embodiments, three prognosis groups are calculated using the risk scores by dividing the patients' risk score into tertiles. In various other embodiments, the prognosis groups are low, intermediate and high risk groups.

In some embodiments, patients in a low risk group have no MSKCC Adverse Clinical Risk Factors, patients in an intermediate risk group have one or two MSKCC Adverse Clinical Risk Factors and patients in a high risk group have more than two MSKCC Adverse Clinical Risk Factors.

In various embodiments, the risk scores are calculated from Table 6 and divided into risk groups.

In various embodiments, the risk scores are divided into equal thirds to generate risk groups for low, intermediate and high risk groups. In other embodiments, the risk scores are divided into percentiles to generate risk groups (higher percentile being better overall survival); for example, 0-40%, 40-60% and 60-100% In another example, the risk scores are divided into high and low risk groups and the median score is used as the cut-off. In another example, the risk scores are divided into high and low risk groups and the mode score is used as the cut-off. In another example, the risk scores are divided into high and low risk groups and the mean score is used as the cut-off.

In other embodiments, overall survival is used to assign risk scores into the risk groups. For example, median survival of 38 months, 21 months and 13 months for low, intermediate, and high risk groups, respectively. Risks scores that correspond with those risk groups are used to predict overall survival. For example, the lower third of the risk scores from Table 6 are patients with median survival of 38 months, the middle third of the risk scores from Table 6 are patients with median survival of 21 months, and top third of the risk scores from Table 6 are patients with median survival of 13 months. Thus, for example, when a subject's risk score is calculated using various methods of the present invention, if the risk score falls within the lower third of the risk scores from Table 6, the patient is considered to have a low risk and a good overall survival (e.g., median overall survival of 38 months).

TABLE 6
normalized Delta Ct expression for each gene for 324 patients.
The higher value, the lower expression of the gene.

| num risk | CRYL1 | TRAF2 | USP6NL | CEP55 | HGF | PCNA | CDK1 | HSD17B10 |
|---|---|---|---|---|---|---|---|---|
| 1-2 | −0.22825 | 2.89275 | 4.98375 | 8.48675 | 4.14275 | 2.07275 | 5.91775 | 2.65875 |
| 1-2 | 4.876917 | 2.37625 | 7.36925 | 9.672917 | 2.84025 | 2.62325 | 8.604917 | 3.45125 |
| 1-2 | −0.02 | 3.796583 | 6.122583 | 8.118 | 3.738583 | 2.569583 | 5.326 | 3.117583 |
| 1-2 | −1.59508 | 2.93925 | 4.84225 | 8.978917 | 4.89625 | 2.91725 | 8.628917 | 2.42225 |
| 0 | 1.733583 | 4.066083 | 6.037083 | 11.42258 | 0.645083 | 2.777083 | 7.529583 | 3.412083 |
| 0 | −1.36358 | 2.864917 | 6.224917 | 9.031417 | 7.045917 | 5.684917 | 9.031417 | 4.420917 |
| 3+ | 2.064083 | 4.199917 | 5.725917 | 8.101083 | 1.844917 | 2.848917 | 5.159083 | 3.058917 |
| 1-2 | −1.48525 | 3.97175 | 5.84975 | 11.15075 | 5.31375 | 3.38675 | 6.13275 | 2.58575 |
| 1-2 | 1.867083 | 3.028917 | 5.852917 | 7.927083 | 4.896917 | 2.284917 | 5.132083 | 2.236917 |
| 1-2 | 0.32925 | 3.197833 | 5.638833 | 7.96125 | 4.883833 | 2.311833 | 5.99325 | 2.864833 |
| 1-2 | −0.28542 | 2.560667 | 7.794667 | 10.73658 | 4.534667 | 4.150667 | 10.95758 | 3.207667 |
| 1-2 | 1.890167 | 3.559667 | 5.604667 | 9.650167 | 2.483667 | 2.374667 | 6.311167 | 2.750667 |
| 0 | −0.00717 | 3.295333 | 4.669333 | 9.711833 | 5.859333 | 1.934333 | 6.032833 | 2.657333 |
| 1-2 | −0.5265 | 2.431833 | 5.496833 | 9.5045 | 4.962833 | 3.483833 | 7.3945 | 3.031833 |
| 1-2 | −0.19167 | 4.220083 | 5.321083 | 9.783333 | 4.573083 | 3.117083 | 7.344333 | 3.010083 |
| 1-2 | −0.10175 | 2.234167 | 8.220167 | 9.25525 | 4.919167 | 2.058167 | 6.65125 | 2.531167 |
| 0 | −1.02817 | 4.128583 | 5.950583 | 11.68983 | 3.823583 | 2.663583 | 6.719833 | 2.537583 |
| 0 | −1.787 | 2.844083 | 6.370083 | 10.274 | 5.463083 | 4.442083 | 10.274 | 3.681083 |
| 1-2 | 0.415917 | 3.097583 | 5.799583 | 8.574917 | 6.249583 | 1.985583 | 7.484917 | 3.494583 |
| 0 | −0.44208 | 4.01575 | 5.85375 | 10.83692 | 3.13175 | 3.65975 | 7.028917 | 2.85475 |
| 1-2 | 3.192333 | 3.80975 | 4.83375 | 7.964333 | 7.75175 | 2.48675 | 6.491333 | 2.52075 |
| 1-2 | 1.188333 | 3.294917 | 5.976917 | 7.595333 | 6.328917 | 2.763917 | 4.805333 | 3.705917 |
| 1-2 | −0.56542 | 3.592417 | 5.510417 | 11.57758 | 4.506417 | 2.982417 | 7.056583 | 2.894417 |
| 1-2 | 1.518333 | 4.885417 | 5.590417 | 7.990333 | 5.116417 | 2.959417 | 5.301333 | 3.081417 |
| 0 | −0.30375 | 2.854333 | 4.782333 | 9.99525 | 4.319333 | 2.670333 | 6.63525 | 2.165333 |
| 1-2 | −0.10258 | 2.926667 | 4.851667 | 13.73442 | 4.392667 | 3.551667 | 7.520417 | 2.769667 |
| 1-2 | −0.16908 | 2.943917 | 5.018917 | 10.80192 | 4.601917 | 2.653917 | 6.692917 | 2.702917 |
| 1-2 | −0.7525 | 3.31325 | 5.95125 | 7.9115 | 6.57425 | 3.34825 | 6.3305 | 2.75225 |
| 1-2 | 1.709417 | 3.679 | 4.892 | 7.310417 | 4.653 | 2.787 | 4.299417 | 2.547 |
| 0 | −0.03 | 3.433 | 5.453 | 9.779 | 4.686 | 3.084 | 6.108 | 3.371 |
| 3+ | −0.87958 | 3.120833 | 6.254833 | 10.90542 | 6.752833 | 4.695833 | 8.454417 | 3.157833 |
| 1-2 | −0.22025 | 2.752583 | 4.268583 | 7.77475 | 4.702583 | 2.331583 | 6.29275 | 2.658583 |
| 1-2 | 1.654917 | 3.000583 | 4.932583 | 6.940917 | 2.636583 | 2.177583 | 5.370917 | 3.144583 |
| 1-2 | 1.349917 | 3.134667 | 4.234667 | 6.559917 | 4.590667 | 1.988667 | 4.582917 | 3.030667 |
| 0 | 0.944833 | 3.030083 | 5.896083 | 8.445833 | 5.011083 | 2.302083 | 5.285833 | 2.428083 |
| 1-2 | 0.383417 | 3.607917 | 5.124917 | 9.185417 | 5.164917 | 1.703917 | 6.196417 | 2.364917 |
| 1-2 | −0.138 | 3.587667 | 5.298667 | 10.206 | 5.351667 | 3.195667 | 7.536 | 3.038667 |
| 1-2 | −0.03075 | 3.252583 | 5.079583 | 8.33825 | 5.840583 | 2.937583 | 6.00025 | 2.086583 |
| 0 | 0.800667 | 3.209417 | 5.299417 | 8.702667 | 4.826417 | 2.558417 | 5.150667 | 2.663417 |
| 1-2 | 3.05575 | 2.528833 | 7.782833 | 7.29275 | 8.539833 | 1.200833 | 5.80975 | 2.061833 |
| 1-2 | −1.71358 | 3.705333 | 7.794333 | 8.179417 | 7.794333 | 3.644333 | 8.179417 | 2.698333 |
| 0 | −0.45342 | 3.160833 | 5.372833 | 10.51558 | 5.382833 | 2.847833 | 6.322583 | 3.006833 |
| 1-2 | 2.3345 | 4.521583 | 6.612583 | 8.8065 | 2.231583 | 3.275583 | 6.7445 | 3.462583 |
| 1-2 | −1.50025 | 2.676333 | 5.006333 | 9.40275 | 5.558333 | 3.809333 | 6.94875 | 2.106333 |
| 0 | −0.07383 | 4.051 | 5.274 | 8.166167 | 6.103 | 2.818 | 6.060167 | 2.527 |
| 1-2 | −2.09767 | 2.685583 | 5.087583 | 9.813333 | 4.897583 | 2.251583 | 7.848333 | 1.707583 |
| 1-2 | 0.988833 | 2.897 | 6.728 | 9.547833 | 5.79 | 4.405 | 8.744833 | 3.97 |
| 1-2 | 0.701083 | 2.819583 | 6.464583 | 10.02608 | 5.377583 | 3.188583 | 7.190083 | 3.748583 |
| 1-2 | 0.331667 | 3.64775 | 4.86675 | 8.206667 | 3.54675 | 2.32075 | 5.487667 | 2.79775 |
| 1-2 | −0.46733 | 3.340667 | 6.047667 | 10.03167 | 6.743667 | 3.134667 | 6.501667 | 2.516667 |
| 1-2 | −0.77467 | 3.40375 | 5.54475 | 10.33033 | 4.82875 | 2.80175 | 7.270333 | 2.87575 |
| 1-2 | 0.73375 | 4.0085 | 6.3005 | 8.76175 | 6.1325 | 3.0935 | 5.41775 | 2.6295 |
| 1-2 | 2.563417 | 2.930583 | 5.675583 | 8.961417 | 3.742583 | 3.062583 | 5.728417 | 3.154583 |
| 1-2 | −1.0195 | 3.688667 | 6.197667 | 10.5365 | 6.898667 | 3.582667 | 6.8285 | 2.180667 |
| 1-2 | −0.42192 | 3.884583 | 5.006583 | 9.418083 | 4.731583 | 2.283583 | 6.725083 | 2.541583 |
| 1-2 | −0.05042 | 2.2665 | 5.4705 | 9.289583 | 1.9245 | 3.2255 | 6.564583 | 3.1175 |
| 0 | −0.40042 | 3.68175 | 6.14775 | 11.38358 | 4.36275 | 3.48275 | 7.041583 | 3.38475 |
| 1-2 | 1.418833 | 3.48475 | 6.16975 | 9.070833 | 4.56875 | 2.72975 | 6.215833 | 2.99075 |
| 1-2 | 0.298333 | 2.6555 | 5.7625 | 11.80233 | 4.7205 | 3.2065 | 6.932333 | 3.0675 |
| 0 | −0.396 | 4.108583 | 6.764583 | 10.192 | 6.291583 | 3.030583 | 7.607 | 3.615583 |
| 1-2 | 0.449833 | 2.0245 | 6.0885 | 10.84383 | 8.1115 | 3.9155 | 8.073833 | 3.6555 |
| 1-2 | 2.6775 | 3.723417 | 5.358417 | 6.3425 | 5.592417 | 2.082417 | 4.4675 | 2.673417 |
| 3+ | 0.604583 | 3.043583 | 6.300583 | 7.711583 | 0.846583 | 2.815583 | 5.239583 | 3.994583 |
| 0 | −0.70592 | 3.14575 | 4.82675 | 9.145083 | 3.81375 | 3.01775 | 7.697083 | 2.72175 |
| 1-2 | 0.419083 | 3.536583 | 4.663583 | 9.433083 | 4.547583 | 2.381583 | 6.588083 | 2.510583 |
| 1-2 | 1.218417 | 4.336917 | 5.945917 | 7.446417 | 5.201917 | 1.592917 | 4.619417 | 2.232917 |
| 1-2 | −0.57242 | 2.601833 | 4.499833 | 7.453583 | 4.595833 | 1.872833 | 5.114583 | 2.629833 |
| 0 | −2.13283 | 2.42 | 5.51 | 10.11717 | 4.203 | 2.61 | 9.385167 | 3.02 |
| 1-2 | 1.546333 | 3.69075 | 5.31075 | 7.457333 | 5.14475 | 2.30675 | 4.690333 | 3.15875 |
| 1-2 | 0.376917 | 3.8755 | 5.0615 | 7.389917 | 4.3965 | 1.4985 | 5.263917 | 2.6575 |
| 1-2 | −1.14017 | 4.16775 | 5.17175 | 9.523833 | 4.63175 | 4.66675 | 9.218833 | 3.14675 |
| 1-2 | 1.652917 | 4.525083 | 4.654083 | 8.858917 | 5.876083 | 2.575083 | 5.882917 | 2.509083 |
| 3+ | −0.59292 | 3.982667 | 5.729667 | 9.845083 | 3.707667 | 3.143667 | 5.409083 | 2.738667 |
| 1-2 | 2.187583 | 3.787 | 5.74 | 6.949583 | 4.842 | 2.448 | 4.652583 | 3.129 |
| 3+ | 1.313417 | 3.5625 | 5.1925 | 7.435417 | 0.4035 | 2.5455 | 3.952417 | 2.3335 |
| 1-2 | −0.57142 | 3.301083 | 4.570083 | 11.51558 | 5.845083 | 3.016083 | 7.363583 | 2.162083 |
| 0 | 0.786083 | 3.423917 | 4.980917 | 7.891083 | 4.259917 | 3.012917 | 7.208083 | 3.325917 |
| 1-2 | −0.20233 | 3.561583 | 6.178583 | 8.025667 | 6.320583 | 1.564583 | 5.190667 | 2.730583 |
| 1-2 | 0.504167 | 3.625667 | 4.575667 | 9.336167 | 2.815667 | 2.043667 | 5.962167 | 2.667667 |
| 1-2 | −0.3885 | 3.3085 | 5.0465 | 12.3605 | 4.6875 | 2.9765 | 7.5955 | 2.8005 |
| 1-2 | −0.34692 | 3.428917 | 4.951917 | 6.058083 | 5.136917 | 1.270917 | 4.139083 | 2.470917 |
| 1-2 | 3.352667 | 3.62075 | 5.39175 | 5.810667 | 2.45075 | 0.85575 | 2.689667 | 2.36275 |
| 3+ | 1.362333 | 3.95675 | 5.55775 | 7.774333 | 0.15775 | 3.52575 | 7.062333 | 3.03775 |
| 1-2 | −0.00692 | 3.252583 | 4.926583 | 8.090083 | 3.882583 | 2.614583 | 5.565083 | 3.015583 |
| 1-2 | 2.5955 | 3.350583 | 5.952583 | 8.9035 | 5.898583 | 2.088583 | 5.3145 | 2.229583 |
| 1-2 | 0.0965 | 3.359833 | 5.256833 | 8.8975 | 5.528833 | 2.543833 | 6.0175 | 2.627833 |
| 1-2 | 0.473583 | 2.965833 | 5.370833 | 7.750583 | 3.109833 | 2.875833 | 5.122583 | 3.218833 |
| 3+ | 0.772167 | 4.908333 | 5.105333 | 8.103167 | 4.935333 | 2.045333 | 5.342167 | 2.610333 |
| 3+ | 1.591583 | 2.85125 | 4.95825 | 7.635583 | −0.33275 | 1.59525 | 5.576583 | 2.66425 |
| 0 | −0.968 | 2.63525 | 5.20425 | 8.772 | 4.92625 | 1.21025 | 5.705 | 2.31825 |
| 1-2 | 0.618333 | 3.844 | 6.219 | 8.661333 | 5.549 | 2.526 | 6.203333 | 2.778 |
| 0 | −0.59083 | 4.318167 | 3.874167 | 8.032167 | 7.207167 | 1.871167 | 5.277167 | 2.194167 |
| 1-2 | 2.925667 | 4.475667 | 6.048667 | 9.146667 | 5.634667 | 4.330667 | 7.743667 | 3.128667 |
| 0 | 1.89775 | 4.2495 | 5.4665 | 8.79775 | 2.2725 | 3.4595 | 6.52075 | 3.4765 |
| 0 | −0.03483 | 3.125667 | 4.850667 | 8.838167 | 4.758667 | 2.611667 | 6.902167 | 2.639667 |
| 1-2 | −0.23308 | 3.663167 | 5.140167 | 10.90592 | 4.120167 | 3.026167 | 6.226917 | 2.848167 |
| 1-2 | −0.40642 | 4.112667 | 6.112667 | 7.134583 | 5.038667 | 2.380667 | 4.169583 | 2.686667 |
| 0 | −0.62242 | 2.782417 | 5.628417 | 11.12258 | 5.131417 | 3.463417 | 6.443583 | 2.989417 |
| 0 | 0.916083 | 2.439417 | 4.595417 | 7.242083 | 5.324417 | 1.444417 | 5.136083 | 1.893417 |
| 0 | −1.43367 | 3.170333 | 5.534333 | 9.238333 | 6.490333 | 5.713333 | 8.169333 | 3.870333 |
| 1-2 | 1.792417 | 3.98675 | 5.70975 | 8.477417 | 3.33175 | 1.24275 | 5.124417 | 2.82175 |
| 0 | −0.22567 | 3.213583 | 5.265583 | 9.576333 | 4.647583 | 2.976583 | 5.971333 | 2.817583 |
| 1-2 | 0.561083 | 3.37275 | 10.19475 | 8.276083 | 8.17475 | 4.00875 | 6.772083 | 4.26175 |
| 1-2 | −1.00117 | 2.0945 | 5.3495 | 9.943833 | 6.0155 | 3.4845 | 9.943833 | 2.9955 |
| 1-2 | −0.44092 | 1.296167 | 6.334167 | 7.619083 | 5.483167 | 3.900167 | 5.705083 | 3.106167 |
| 1-2 | 0.737083 | 3.98 | 5.402 | 8.861083 | 5.084 | 2.57 | 5.137083 | 2.905 |
| 1-2 | −0.94783 | 2.8435 | 5.5745 | 9.726167 | 6.5745 | 3.4465 | 7.092167 | 2.6015 |
| 1-2 | −0.652 | 3.797083 | 6.823083 | 9.007 | 4.489083 | 2.904083 | 7.45 | 2.599083 |
| 3+ | 1.913083 | 4.413167 | 6.140167 | 8.340083 | 4.040167 | 2.501167 | 5.498083 | 3.369167 |
| 0 | 0.02425 | 3.5335 | 4.7285 | 12.06225 | 3.8505 | 2.4935 | 6.86425 | 2.4455 |
| 1-2 | 1.823583 | 3.87425 | 4.85325 | 7.853583 | 5.07425 | 2.71725 | 5.791583 | 3.37925 |
| 1-2 | 0.67875 | 2.915167 | 6.578167 | 8.33375 | 8.922167 | 3.592167 | 7.67675 | 3.303167 |
| 0 | −0.45242 | 3.630583 | 5.672583 | 8.689583 | 5.433583 | 6.592583 | 7.632583 | 2.820583 |
| 0 | 1.797833 | 2.957917 | 5.909917 | 8.052833 | 3.167917 | 2.496917 | 4.719833 | 2.981917 |
| 1-2 | 0.503333 | 3.7785 | 5.9495 | 9.503333 | 5.6585 | 2.7835 | 6.490333 | 3.3905 |
| 1-2 | 1.446083 | 3.490583 | 4.970583 | 7.339083 | 5.651583 | 2.477583 | 5.284083 | 3.318583 |
| 1-2 | −0.95017 | 4.064917 | 5.280917 | 10.76083 | 2.824917 | 2.170917 | 6.558833 | 2.314917 |
| 1-2 | 2.004583 | 3.783333 | 5.553333 | 9.620583 | 4.116333 | 2.293333 | 6.647583 | 2.985333 |
| 1-2 | 0.678583 | 3.07 | 5.151 | 10.21258 | 3.6 | 3.389 | 7.730583 | 3.281 |
| 0 | −0.33958 | 3.59475 | 5.99475 | 11.03242 | 5.05975 | 3.70975 | 8.860417 | 3.08875 |
| 1-2 | −1.14458 | 2.731333 | 5.079333 | 10.43442 | 4.774333 | 3.765333 | 5.744417 | 2.281333 |
| 1-2 | −0.51883 | 2.849167 | 6.488167 | 10.08117 | 6.246167 | 4.276167 | 6.171167 | 3.821167 |
| 1-2 | −0.12542 | 3.485833 | 9.963833 | 9.934583 | 9.963833 | 5.991833 | 9.934583 | 3.974833 |
| 1-2 | −1.62108 | 1.663583 | 7.329583 | 11.24192 | 8.173583 | 3.833583 | 6.475917 | 2.784583 |
| 1-2 | 1.7695 | 2.689917 | 5.493917 | 12.3045 | 6.021917 | 2.891917 | 8.4565 | 2.466917 |
| 0 | 0.486333 | 3.353833 | 4.800833 | 9.849333 | 3.920833 | 2.154833 | 6.201333 | 2.670833 |
| 1-2 | 1.323083 | 3.49225 | 5.22825 | 9.054083 | 4.55725 | 2.84725 | 6.212083 | 3.14425 |
| 1-2 | −1.81067 | 1.822417 | 8.865417 | 10.22233 | 5.664417 | 5.479417 | 7.043333 | 3.233417 |
| 1-2 | 1.443917 | 3.79325 | 6.94625 | 8.502917 | 7.27925 | 3.60125 | 5.630917 | 2.81925 |
| 1-2 | 1.781833 | 4.355917 | 6.054917 | 6.858833 | −1.34208 | 2.398917 | 4.794833 | 2.521917 |
| 1-2 | 0.580833 | 3.875167 | 6.092167 | 11.41383 | 4.999167 | 3.045167 | 6.690833 | 3.389167 |
| 1-2 | 1.0045 | 3.596583 | 5.998583 | 9.3455 | 7.290583 | 4.302583 | 5.8765 | 3.097583 |
| 1-2 | −1.234 | 3.443333 | 4.949333 | 9.492 | 7.646333 | 3.972333 | 6.621 | 3.486333 |
| 0 | 1.214333 | 3.575417 | 5.096417 | 10.34333 | 5.341417 | 1.865417 | 5.942333 | 2.735417 |
| 3+ | 0.537417 | 3.453417 | 5.254417 | 7.406417 | 5.858417 | 0.877417 | 4.380417 | 2.457417 |
| 0 | 0.331167 | 3.7265 | 4.9955 | 8.735167 | 4.7355 | 2.7015 | 5.647167 | 2.8775 |
| 0 | −1.15383 | 3.231833 | 5.058833 | 8.240167 | 11.02083 | 2.889833 | 7.029167 | 2.254833 |
| 1-2 | −0.175 | 3.073 | 7.676 | 10.37 | 6.202 | 2.527 | 6.161 | 3.347 |
| 1-2 | 1.703 | 2.900833 | 5.288833 | 6.936 | 4.201833 | 2.719833 | 5.359 | 3.424833 |
| 1-2 | 1.929667 | 3.857833 | 5.590833 | 8.437667 | −0.20517 | 2.256833 | 5.016667 | 3.223833 |
| 1-2 | −0.93942 | 2.945417 | 8.079417 | 9.232583 | 5.327417 | 4.609417 | 9.232583 | 3.827417 |
| 0 | 0.456167 | 2.95675 | 4.61275 | 8.724167 | 2.71575 | 2.11875 | 7.708167 | 2.93175 |
| 1-2 | 0.210333 | 3.345833 | 4.552833 | 7.650333 | 1.817833 | 2.631833 | 4.931333 | 2.193833 |
| 1-2 | 0.14675 | 3.610167 | 5.288167 | 8.37275 | 5.651167 | 2.390167 | 6.66075 | 2.938167 |
| 1-2 | 0.102667 | 1.969583 | 7.214583 | 8.545667 | 4.857583 | 4.839583 | 5.404667 | 2.388583 |
| 1-2 | 1.403583 | 3.1845 | 6.9315 | 8.759583 | 1.0645 | 2.8935 | 5.277583 | 2.9115 |
| 1-2 | 2.191667 | 3.216333 | 4.163333 | 6.745667 | 1.268333 | 1.610333 | 4.421667 | 3.197333 |
| 1-2 | −0.40217 | 3.739167 | 5.756167 | 9.578833 | 6.813167 | 3.125167 | 6.327833 | 2.888167 |
| 1-2 | −0.64292 | 3.009667 | 5.710667 | 7.912083 | 4.023667 | 2.875667 | 5.993083 | 2.233667 |
| 0 | 1.291583 | 3.831917 | 5.674917 | 7.538583 | 4.284917 | 2.895917 | 5.702583 | 2.644917 |
| 1-2 | −1.08933 | 3.33375 | 7.50475 | 9.498667 | 9.39075 | 4.32575 | 7.625667 | 2.55475 |
| 0 | −0.24558 | 2.71625 | 4.52425 | 11.29442 | 3.93325 | 1.92125 | 6.308417 | 2.32525 |
| 3+ | −0.11483 | 3.917083 | 6.026083 | 9.125167 | 12.45808 | 3.399083 | 8.676167 | 2.730083 |
| 1-2 | −0.70367 | 7.865667 | 8.777667 | 10.18333 | 9.685667 | 3.468667 | 10.18333 | 3.033667 |
| 1-2 | 2.865667 | 3.359833 | 6.035833 | 7.049667 | −1.57817 | 2.260833 | 5.224667 | 3.584833 |
| 1-2 | 2.116167 | 3.988083 | 4.784083 | 5.672167 | −0.58092 | 2.370083 | 3.647167 | 2.938083 |
| 1-2 | 0.662167 | 3.024417 | 6.775417 | 7.452167 | 3.222417 | 3.330417 | 5.588167 | 3.400417 |
| 0 | 0.1475 | 3.559917 | 4.834917 | 8.5525 | 3.709917 | 2.783917 | 6.3665 | 2.512917 |
| 0 | −1.14708 | 4.021417 | 5.239417 | 8.681917 | 4.050417 | 2.454417 | 6.288917 | 2.512417 |
| 1-2 | 1.34425 | 3.451167 | 5.128167 | 6.84125 | 4.005167 | 2.508167 | 4.85425 | 2.681167 |
| 1-2 | 0.04125 | 3.181417 | 5.206417 | 8.85025 | 4.823417 | 2.494417 | 6.29625 | 2.485417 |
| 0 | −0.3815 | 3.598167 | 6.083167 | 11.3745 | 4.875167 | 3.508167 | 6.7965 | 2.977167 |
| 1-2 | 1.766917 | 3.005 | 5.608 | 7.246917 | 6.418 | 3.564 | 5.707917 | 3.518 |
| 1-2 | −1.18625 | 2.945417 | 5.126417 | 8.93775 | 4.646417 | 8.669417 | 6.22875 | 3.211417 |
| 0 | −0.56458 | 3.063 | 5.862 | 8.789417 | 9.529 | 3.392 | 6.667417 | 3.067 |
| 1-2 | 2.944583 | 3.813417 | 5.344417 | 7.039583 | −1.40858 | 2.426417 | 4.512583 | 3.171417 |
| 1-2 | −0.1885 | 3.10975 | 5.04375 | 9.7855 | 4.88675 | 3.26175 | 8.6815 | 3.68675 |
| 1-2 | −2.39283 | 1.609083 | 6.278083 | 8.468167 | 4.853083 | 3.893083 | 4.823167 | 3.402083 |
| 1-2 | −1.13692 | 3.375917 | 6.503917 | 10.70608 | 7.410917 | 2.813917 | 8.980083 | 3.160917 |
| 1-2 | −0.81083 | 3.037583 | 8.841583 | 8.860167 | 8.841583 | 2.642583 | 7.115167 | 2.605583 |
| 1-2 | −1.57308 | 2.488833 | 4.423833 | 10.68092 | 3.632833 | 1.873833 | 6.699917 | 2.301833 |
| 1-2 | −0.13017 | 4.34125 | 5.49125 | 8.603833 | 5.58025 | 3.74325 | 7.039833 | 2.85525 |
| 3+ | −0.36175 | 2.755 | 5.045 | 8.51625 | 4.714 | 3.57 | 6.11025 | 3.045 |
| 1-2 | −0.76042 | 3.640583 | 5.291583 | 9.202583 | 5.451583 | 2.616583 | 5.841583 | 2.964583 |
| 1-2 | 0.500417 | 3.79825 | 6.66625 | 8.949417 | 5.69025 | 3.52625 | 7.573417 | 3.13425 |
| 1-2 | 0.185 | 2.8415 | 8.1975 | 10.439 | 7.0905 | 3.7315 | 6.091 | 3.4495 |
| 0 | 1.00775 | 3.37125 | 5.61825 | 8.99375 | 4.99125 | 2.87125 | 6.72275 | 2.99225 |
| 1-2 | 0.4885 | 4.05 | 5.455 | 6.9115 | 6.071 | 1.931 | 3.4505 | 2.569 |
| 0 | 0.226833 | 2.829 | 5.052 | 7.789833 | 2.517 | 1.786 | 5.164833 | 2.618 |
| 1-2 | 1.998167 | 2.698167 | 5.146167 | 8.562167 | 2.436167 | 2.715167 | 4.667167 | 2.429167 |
| 1-2 | 0.909333 | 1.91175 | 5.19775 | 9.804333 | −0.08925 | 3.37075 | 6.954333 | 3.31075 |
| 0 | −1.12758 | 3.297333 | 7.357333 | 9.393417 | 9.488333 | 3.638333 | 8.072417 | 3.964333 |
| 1-2 | −0.78058 | 3.9045 | 5.6235 | 9.855417 | 3.9085 | 3.3965 | 5.715417 | 3.0765 |
| 1-2 | 3.04175 | 4.328917 | 4.265917 | 6.68675 | 4.587917 | 1.889917 | 3.52975 | 2.627917 |
| 1-2 | −1.64192 | 3.696667 | 7.271667 | 9.604083 | 5.046667 | 4.573667 | 8.994083 | 3.800667 |
| 1-2 | −0.364 | 3.945333 | 5.128333 | 9.107 | 4.649333 | 2.219333 | 7.203 | 2.585333 |
| 1-2 | 0.105833 | 3.113083 | 6.778083 | 12.08083 | 5.538083 | 3.606083 | 5.994833 | 3.289083 |
| 1-2 | 1.07325 | 3.921 | 5.75 | 8.25925 | −0.908 | 2.706 | 4.68325 | 2.346 |
| 1-2 | −0.58717 | 3.546583 | 6.630583 | 8.202833 | 7.155583 | 1.986583 | 5.650833 | 2.870583 |
| 3+ | 0.034333 | 2.75 | 4.855 | 8.686333 | 5.007 | 2.224 | 5.914333 | 2.913 |
| 1-2 | 0.815083 | 3.671167 | 6.524167 | 9.139083 | 5.183167 | 3.119167 | 6.179083 | 3.079167 |
| 0 | 0.506667 | 3.0345 | 5.7365 | 8.300667 | 5.2165 | 3.0365 | 6.501667 | 2.8575 |
| 0 | 0.760333 | 3.324833 | 6.263833 | 9.179333 | 5.423833 | 3.011833 | 6.883333 | 3.411833 |
| 0 | −0.466 | 3.340167 | 7.575167 | 8.993 | 7.079167 | 3.286167 | 5.784 | 3.478167 |
| 1-2 | 0.9865 | 3.478 | 7.202 | 9.6005 | 2.898 | 2.784 | 6.2715 | 1.672 |
| 3+ | −0.0945 | 3.791583 | 6.414583 | 11.5085 | 8.310583 | 4.056583 | 7.7395 | 3.227583 |
| 0 | −0.75925 | 2.505083 | 8.711083 | 8.64375 | 4.020083 | 4.423083 | 8.64375 | 3.970083 |
| 1-2 | 1.414167 | 3.015417 | 5.729417 | 10.87517 | 6.479417 | 3.676417 | 6.405167 | 3.160417 |
| 1-2 | 0.065667 | 3.328083 | 5.215083 | 7.500667 | 3.724083 | 2.463083 | 4.792667 | 2.581083 |
| 1-2 | 1.012833 | 3.265333 | 6.336333 | 8.765833 | 3.242333 | 2.404333 | 5.559833 | 2.930333 |
| 0 | −2.62292 | 2.123083 | 7.247083 | 8.251083 | 8.180083 | 6.150083 | 7.332083 | 4.504083 |
| 1-2 | 2.692 | 3.590417 | 5.685417 | 7.473 | 2.636417 | 2.142417 | 5.37 | 3.096417 |
| 1-2 | 0.309917 | 2.554667 | 6.758667 | 9.969917 | 6.043667 | 4.806667 | 7.712917 | 3.416667 |
| 1-2 | 1.6665 | 3.9395 | 5.7555 | 10.1255 | 4.6865 | 3.2405 | 8.2295 | 3.5015 |
| 0 | 0.681583 | 3.88575 | 5.05875 | 9.897583 | 5.05275 | 1.64675 | 7.024583 | 2.19375 |
| 1-2 | 1.107 | 2.995 | 6.511 | 8.808 | 4.427 | 2.939 | 6.672 | 3.088 |
| 1-2 | 0.403583 | 2.91275 | 6.80975 | 8.203583 | 10.69075 | 4.65275 | 6.518583 | 3.04175 |
| 1-2 | 3.934333 | 2.593167 | 5.064167 | 6.034333 | −1.81883 | 2.238167 | 3.021333 | 3.616167 |
| 1-2 | 3.770167 | 2.84275 | 4.63275 | 5.140167 | 3.58075 | 1.72675 | 3.769167 | 2.85175 |
| 0 | 1.304083 | 3.8975 | 6.2715 | 7.975083 | 5.4875 | 2.0885 | 6.200083 | 3.7355 |
| 1-2 | 0.12525 | 3.42375 | 4.67175 | 9.76025 | 4.15775 | 3.98075 | 7.22425 | 3.17875 |
| 0 | −0.22208 | 3.603833 | 7.444833 | 8.980917 | 3.329833 | 2.796833 | 5.954917 | 2.774833 |
| 0 | −0.80258 | 3.29625 | 7.33925 | 11.75842 | 6.82325 | 4.09725 | 10.06242 | 4.24625 |
| 1-2 | 0.89425 | 4.3315 | 6.3775 | 10.45325 | 6.2535 | 3.2355 | 6.25925 | 3.3695 |
| 3+ | 0.963417 | 3.877083 | 5.590083 | 9.758417 | 1.805083 | 3.877083 | 6.226417 | 4.561083 |
| 1-2 | 0.19375 | 3.76175 | 6.23175 | 11.06075 | 5.59675 | 2.52875 | 7.48075 | 3.14775 |
| 1-2 | −1.94817 | 2.961083 | 6.004083 | 10.51483 | 7.200083 | 2.952083 | 5.888833 | 2.510083 |
| 1-2 | 1.687833 | 3.014333 | 4.921333 | 7.439833 | 4.578333 | 2.702333 | 5.112833 | 2.885333 |
| 3+ | 1.374333 | 3.721167 | 5.818167 | 5.123333 | 4.822167 | 1.526167 | 5.025333 | 1.612167 |
| 0 | −0.02725 | 3.25 | 5.21 | 9.11175 | 4.443 | 2.748 | 7.00275 | 2.617 |
| 3+ | 0.03875 | 4.426667 | 5.792667 | 7.18075 | 0.720667 | 2.405667 | 5.33875 | 3.855667 |
| 1-2 | 1.929333 | 3.252417 | 5.300417 | 11.00633 | 4.922417 | 3.733417 | 7.638333 | 3.346417 |
| 1-2 | −0.78367 | 2.919667 | 6.292667 | 11.01233 | 5.809667 | 3.036667 | 7.922333 | 3.010667 |
| 1-2 | −0.14392 | 2.78375 | 5.86775 | 9.417083 | 3.75475 | 2.66775 | 6.826083 | 2.45775 |
| 0 | 0.404083 | 3.723917 | 4.506917 | 9.463083 | 3.786917 | 1.991917 | 6.297083 | 2.763917 |
| 1-2 | −0.11367 | 4.775833 | 5.099833 | 8.553333 | 4.738833 | 2.685833 | 6.512333 | 3.046833 |
| 1-2 | −0.25 | 3.245 | 8.704 | 6.987 | 4.842 | 2.472 | 8.358 | 3.029 |
| 0 | 1.1745 | 2.4465 | 5.2115 | 7.3645 | 4.4445 | 1.8535 | 5.7195 | 3.3855 |
| 0 | 0.257667 | 4.415 | 4.751 | 9.222667 | 5.13 | 2.363 | 5.495667 | 2.67 |
| 0 | −1.49375 | 3.0985 | 4.5855 | 8.33525 | 4.6805 | 2.5485 | 5.56625 | 2.4595 |
| 1-2 | −1.22233 | 2.837917 | 5.903917 | 9.611667 | 4.004917 | 3.549917 | 9.611667 | 2.974917 |
| 1-2 | 0.14725 | 2.719167 | 4.491167 | 6.59025 | 0.997167 | 1.138167 | 5.42325 | 2.502167 |
| 0 | −1.53858 | 4.180417 | 6.222417 | 9.530417 | 6.856417 | 2.740417 | 7.548417 | 2.932417 |
| 1-2 | −0.74333 | 2.355 | 5.263 | 9.302667 | 5.635 | 1.62 | 5.969667 | 1.929 |
| 1-2 | 0.778083 | 2.954083 | 5.044083 | 9.672083 | 6.964083 | 3.120083 | 5.824083 | 2.795083 |
| 1-2 | 1.594833 | 2.78275 | 4.52075 | 7.909833 | 4.86575 | 2.47975 | 5.720833 | 2.28975 |
| 0 | −1.30408 | 3.134 | 7.521 | 9.424917 | 6.317 | 2.122 | 7.732917 | 2.763 |
| 0 | 1.24 | 3.882667 | 4.499667 | 9.872 | 3.416667 | 2.931667 | 6.423 | 2.929667 |
| 0 | −0.42367 | 2.198 | 4.829 | 6.212333 | 4.148 | 1.894 | 4.453333 | 2.129 |
| 1-2 | −2.11592 | 3.606417 | 5.046417 | 8.633083 | 5.209417 | 3.469417 | 4.833083 | 3.206417 |
| 0 | 0.83125 | 2.8575 | 6.7455 | 9.43025 | 5.9105 | 3.8365 | 6.16225 | 3.1945 |
| 1-2 | −1.73967 | 3.441833 | 7.599833 | 11.83233 | 6.956833 | 3.423833 | 7.612333 | 3.222833 |
| 1-2 | 1.718083 | 3.32725 | 5.52525 | 10.34908 | 4.42925 | 3.29525 | 7.242083 | 2.98425 |
| 1-2 | 0.661333 | 3.339917 | 4.440917 | 7.887333 | 4.204917 | 1.755917 | 4.701333 | 2.815917 |
| 0 | 0.275417 | 3.554167 | 6.357167 | 9.425417 | 4.175167 | 2.593167 | 6.663417 | 2.854167 |
| 1-2 | 0.759167 | 3.419833 | 5.938833 | 8.704167 | 4.763833 | 2.106833 | 4.935167 | 2.755833 |
| 1-2 | −1.59425 | 2.69825 | 6.25625 | 11.10275 | 6.27125 | 3.15325 | 11.10275 | 2.69625 |
| 1-2 | −1.4545 | 2.061667 | 5.115667 | 11.0305 | 4.682667 | 3.108667 | 11.0305 | 2.590667 |
| 1-2 | 0.324333 | 3.528333 | 5.479333 | 8.669333 | 4.390333 | 2.639333 | 6.013333 | 3.222333 |
| 1-2 | 0.681917 | 3.916583 | 4.699583 | 8.019917 | 6.017583 | 2.213583 | 4.939917 | 2.926583 |
| 1-2 | 1.33875 | 3.261917 | 5.743917 | 7.02075 | 5.421917 | 1.959917 | 5.04975 | 3.069917 |
| 1-2 | 0.0255 | 2.784333 | 6.849333 | 8.6255 | 6.963333 | 2.060333 | 6.8615 | 3.141333 |
| 1-2 | 3.8525 | 1.899667 | 8.292667 | 8.5405 | 8.292667 | 3.384667 | 4.8925 | 4.285667 |
| 1-2 | 1.471 | 3.687917 | 5.139917 | 8.454 | 1.867917 | 4.041917 | 6.209 | 3.334917 |
| 0 | 0.487667 | 3.742417 | 7.465417 | 10.33567 | 6.417417 | 3.105417 | 6.766667 | 3.036417 |
| 1-2 | −0.39967 | 2.730333 | 5.896333 | 9.954333 | 1.799333 | 3.001333 | 7.054333 | 3.026333 |
| 0 | 0.81775 | 3.521667 | 5.239667 | 9.11675 | 2.006667 | 2.278667 | 6.38475 | 2.846667 |
| 3+ | −1.83033 | 3.744083 | 6.389083 | 9.242667 | 9.121083 | 4.965083 | 8.379667 | 2.899083 |
| 3+ | 0.778083 | 3.643417 | 5.852417 | 10.72708 | 7.674417 | 3.535417 | 10.72708 | 3.193417 |
| 1-2 | −0.05175 | 3.088833 | 4.998833 | 11.17225 | 4.432833 | 2.487833 | 6.96825 | 2.100833 |
| 1-2 | −0.11642 | 2.616917 | 6.164917 | 9.280583 | 6.365917 | 3.307917 | 7.726583 | 2.748917 |
| 1-2 | 0.65475 | 2.53775 | 4.63675 | 7.88975 | 3.57575 | 1.89275 | 5.70975 | 2.65375 |
| 1-2 | 1.7655 | 3.7475 | 4.6295 | 7.9045 | 5.9485 | 1.9125 | 4.8495 | 3.5505 |
| 0 | 0.4245 | 2.740667 | 4.868667 | 9.6605 | 4.288667 | 2.514667 | 6.3545 | 2.690667 |
| 1-2 | −0.29208 | 3.44725 | 5.42425 | 8.869917 | 5.64525 | 2.75125 | 5.864917 | 2.70825 |
| 1-2 | 0.293917 | 3.601833 | 5.404833 | 9.064917 | 4.506833 | 3.238833 | 7.166917 | 3.060833 |
| 0 | −1.64058 | 1.85625 | 6.27925 | 9.980417 | 4.69325 | 4.24725 | 9.980417 | 3.57525 |
| 1-2 | 1.200833 | 1.900167 | 7.437167 | 9.433833 | 8.787167 | 2.139167 | 7.336833 | 3.673167 |
| 0 | −1.1235 | 2.859167 | 4.405167 | 8.6815 | 5.190167 | 1.538167 | 5.6445 | 1.989167 |
| 1-2 | 0.00025 | 3.29575 | 5.13475 | 10.23325 | 5.24875 | 2.96275 | 6.22925 | 3.01575 |
| 1-2 | 0.33775 | 3.028417 | 6.681417 | 10.36375 | 7.310417 | 3.145417 | 6.96275 | 2.896417 |
| 0 | 0.16275 | 3.071917 | 4.543917 | 7.96275 | 3.271917 | 1.849917 | 5.90275 | 2.234917 |
| 1-2 | 1.703 | 3.072667 | 5.267667 | 8.571 | 0.105667 | 2.927667 | 5.746 | 2.947667 |
| 1-2 | 2.1175 | 3.26175 | 4.78975 | 5.5465 | 2.21175 | 1.83075 | 3.8325 | 3.08975 |
| 3+ | 2.79525 | 0.724667 | 4.031667 | 11.40625 | 4.443667 | 0.897667 | 4.51725 | 1.028667 |
| 1-2 | 1.265083 | 2.736667 | 6.172667 | 8.968083 | 6.422667 | 2.483667 | 7.319083 | 3.589667 |
| 0 | 0.28725 | 3.484 | 5.498 | 8.62725 | 6.542 | 2.818 | 6.54725 | 2.255 |
| 1-2 | 1.159583 | 3.70275 | 5.04275 | 8.548583 | 3.67975 | 2.40675 | 5.806583 | 2.83575 |
| 3+ | 1.436917 | 3.08575 | 7.14275 | 8.996917 | 6.49275 | 4.75375 | 8.012917 | 3.82375 |
| 1-2 | 1.493333 | 3.681083 | 4.671083 | 8.089333 | −0.12792 | 1.793083 | 5.435333 | 2.881083 |
| 1-2 | 1.053667 | 2.642333 | 5.653333 | 10.12767 | 6.145333 | 4.566333 | 5.982667 | 3.352333 |
| 1-2 | 1.1105 | 3.850083 | 5.820083 | 9.0785 | 4.590083 | 3.385083 | 6.2705 | 2.905083 |
| 0 | 0.092667 | 3.333833 | 4.377833 | 10.86067 | 3.956833 | 2.019833 | 7.436667 | 2.756833 |
| 0 | 0.483833 | 3.328167 | 4.885167 | 8.380833 | 5.080167 | 2.595167 | 5.636833 | 3.305167 |
| 1-2 | 0.056083 | 3.467 | 4.882 | 7.654083 | 4.196 | 2.22 | 5.195083 | 2.663 |
| 0 | −0.87692 | 4.132667 | 6.595667 | 11.15908 | 5.765667 | 3.332667 | 8.996083 | 2.775667 |
| 3+ | 3.440333 | 3.74375 | 5.24175 | 6.540333 | 3.20575 | 2.19675 | 3.943333 | 3.19175 |
| 0 | −0.32625 | 3.793 | 5.677 | 8.58475 | 5.252 | 2.914 | 6.31375 | 2.787 |
| 1-2 | −0.29033 | 3.489917 | 4.696917 | 11.66867 | 5.964917 | 3.019917 | 7.094667 | 3.106917 |
| 1-2 | −0.02867 | 3.056083 | 5.089083 | 9.918333 | 5.226083 | 2.838083 | 6.677333 | 2.143083 |
| 3+ | 0.96 | 3.657917 | 6.031917 | 10.674 | 6.369917 | 3.171917 | 7.009 | 2.793917 |
| 1-2 | −0.40633 | 2.644667 | 5.287667 | 8.503667 | 4.764667 | 2.541667 | 6.492667 | 2.717667 |
| 1-2 | 0.0255 | 3.299833 | 5.303833 | 11.0525 | 4.437833 | 2.486833 | 7.7475 | 2.535833 |
| 0 | −1.03292 | 2.574583 | 5.254583 | 8.621083 | 5.087583 | 2.566583 | 5.793083 | 3.153583 |
| 1-2 | 1.992167 | 2.870833 | 4.968833 | 6.720167 | −0.50817 | 2.049833 | 3.905167 | 2.784833 |
| 1-2 | −0.07858 | 3.940917 | 4.717917 | 11.29442 | 4.655917 | 2.673917 | 7.354417 | 2.743917 |
| 3+ | −1.04292 | 2.305 | 5.366 | 11.73308 | 5.827 | 3.54 | 7.163083 | 3.515 |
| 0 | −1.09433 | 4.125333 | 5.073333 | 9.820667 | 10.27333 | 2.820333 | 8.145667 | 3.373333 |
| 1-2 | 0.137917 | 3.508583 | 5.017583 | 9.052917 | 4.503583 | 2.806583 | 6.354917 | 3.324583 |
| 1-2 | 0.474583 | 3.741833 | 4.777833 | 8.199583 | 4.835833 | 2.248833 | 6.072583 | 2.711833 |
| 1-2 | −0.22975 | 2.966917 | 4.469917 | 9.26425 | 5.681917 | 1.906917 | 6.59525 | 2.386917 |
| 1-2 | 2.331667 | 2.278583 | 6.936583 | 6.306667 | 5.277583 | 2.667583 | 4.688667 | 3.124583 |
| 1-2 | −0.62283 | 3.179917 | 5.435917 | 10.62417 | 4.082917 | 3.720917 | 7.410167 | 2.905917 |
| 3+ | −0.30042 | 1.886167 | 6.547167 | 11.36958 | 6.095167 | 3.038167 | 11.36958 | 3.162167 |
| 1-2 | 0.739917 | 2.978083 | 4.989083 | 8.369917 | 3.909083 | 2.772083 | 4.999917 | 2.391083 |
| 3+ | −0.09992 | 2.950083 | 5.167083 | 9.021083 | 5.456083 | 2.658083 | 6.296083 | 2.812083 |
| 1-2 | 0.734417 | 3.219833 | 6.390833 | 9.500417 | 5.489833 | 2.350833 | 6.746417 | 2.515833 |
| 0 | 0.945917 | 3.1125 | 4.9845 | 7.899917 | 4.7295 | 2.3235 | 5.117917 | 2.8975 |
| 1-2 | −0.13117 | 3.8515 | 6.5385 | 9.470833 | 8.0875 | 3.1165 | 7.426833 | 3.3285 |
| 1-2 | −1.02717 | 3.049917 | 7.963917 | 10.19583 | 6.885917 | 4.030917 | 8.281833 | 3.458917 |
| 1-2 | 0.140167 | 4.302 | 5.451 | 9.352167 | 3.816 | 2.906 | 6.411167 | 2.921 |
| 0 | 1.724167 | 3.019417 | 4.364417 | 6.062167 | −1.76958 | 1.133417 | 4.235167 | 3.338417 |
| 1-2 | −0.26442 | 2.004167 | 4.127167 | 9.131583 | 3.954167 | 2.233167 | 5.960583 | 2.649167 |
| 1-2 | 0.3205 | 2.48325 | 6.12625 | 9.2245 | 6.15425 | 2.76925 | 6.2105 | 3.27225 |
| 1-2 | 0.125333 | 2.808333 | 4.623333 | 11.72033 | 4.138333 | 2.758333 | 6.668333 | 2.876333 |
| 0 | 0.718583 | 3.90525 | 4.94625 | 8.199583 | 4.32125 | 2.80625 | 5.877583 | 3.28425 |
| 1-2 | 0.3185 | 3.093083 | 6.159083 | 9.1645 | 1.979083 | 3.592083 | 5.9795 | 3.367083 |
| 1-2 | 0.876917 | 3.013 | 5.684 | 9.713917 | 2.025 | 3.05 | 6.893917 | 3.097 |
| 0 | 1.207 | 3.5055 | 4.9445 | 8.962 | 3.5605 | 2.5025 | 5.755 | 2.8555 |
| 0 | 0.3635 | 3.045083 | 5.314083 | 9.0155 | 6.543083 | 3.196083 | 5.9155 | 3.036083 |
| 0 | 1.130583 | 3.6085 | 5.7335 | 8.852583 | 5.5395 | 3.5425 | 6.575583 | 3.3285 |
| 1-2 | 1.360417 | 1.713417 | 3.937417 | 6.253417 | 4.793417 | 1.581417 | 3.643417 | 2.460417 |
| 1-2 | −0.20592 | 2.207583 | 5.635583 | 10.20108 | 5.068583 | 2.954583 | 6.789083 | 3.116583 |
| 0 | −1.44308 | 3.623417 | 6.546417 | 9.828917 | 5.613417 | 3.808417 | 6.683917 | 2.754417 |

### Gene Expression Level Determination

In various embodiments, the metastatic clear cell renal cell carcinoma gene and reference gene expression level is determined using quantitative polymerase chain reaction (qPCR), using a specific primer sequence and/or a probe sequence. In various embodiments, the primer sequence or probe sequence used for the metastatic clear cell renal cell carcinoma gene and the reference gene are:

CDK1:
(SEQ ID NO: 1)
ACCTATGGAGTTGTGTATAAGGGTAGAC,
(SEQ ID NO: 2)
ACCCCTTCCTCTTCACTTTCTAGT
and
(SEQ ID NO: 3)
CATGGCTACCACTTGACC;
CEP55:
(SEQ ID NO: 4)
CTCCAAACTGCTTCAACTCATCAAT,
(SEQ ID NO: 5)
ACACGAGCCACTGCTGATTTT
and
(SEQ ID NO: 6)
CTCCAGAGCATCTTTC;
CRYL1:
(SEQ ID NO: 7)
CGTTGGCAGTGGAGTCATTG,
(SEQ ID NO: 8)
GGAAGCCTCCACTGGCAAA
and
(SEQ ID NO: 9)
ATGGCCCAGCTTCGCC;
HGF:
(SEQ ID NO: 10)
CATTCACTTGCAAGGCTTTTGTTTT,
(SEQ ID NO: 11)
TTTCACTCCACTTGACATGCTATTGA
and
(SEQ ID NO: 12)
AACAATGCCTCTGGTTCCC;
HSD17B10:
(SEQ ID NO: 13)
CCAAGCCAAGAAGTTAGGAAACAAC,
(SEQ ID NO: 14)
GCTGTTTGCACATCCTTCTCAGA
and
(SEQ ID NO: 15)
CCCAGCCGACGTGACC;
PCNA:
(SEQ ID NO: 16)
TGAACCTCACCAGTATGTCCAAAAT,
(SEQ ID NO: 17)
CGTTATCTTCGGCCCTTAGTGTAAT
and
(SEQ ID NO: 18)
CCGGCGCATTTTAGT;
TRAF2:
(SEQ ID NO: 19)
GGAAGCGCCAGGAAGCT,
(SEQ ID NO: 20)
CCGTACCTGCTGGTGTAGAAG
and
(SEQ ID NO: 21)
ATACCCGCCATCTTCT;
USP6NL:
(SEQ ID NO: 22)
GAGGAGCTCCCAGATCATAATGTG,
(SEQ ID NO: 23)
GCATTTTCAGCCATTTGGTAGTTCT
and
(SEQ ID NO: 24)
AAGCACCTGGAAATTG;
ACTB:
(SEQ ID NO: 25)
CCAGCTCACCATGGATGATG,
(SEQ ID NO: 26)
ATGCCGGAGCCGTTGTC
and
(SEQ ID NO: 27)
TCGCCGCGCTCGTC;
GUSB:
(SEQ ID NO: 28)
CTCATTTGGAATTTTGCCGATT,
(SEQ ID NO: 29)
CCGAGTGAAGATCCCCTTTTTA
and
(SEQ ID NO: 30)
TCACCGACGAGAGTGC;
HPRT1:
(SEQ ID NO: 31)
ATGGACAGGACTGAACGTCTTG,
(SEQ ID NO: 32)
GCACACAGAGGGCTACAATGT
and
(SEQ ID NO: 33)
CCTCCCATCTCCTTCATCA;
RPL13A:
(SEQ ID NO: 34)
ACCAACCCTTCCCGAGGC,
(SEQ ID NO: 35)
TTGGTTTTGTGGGGCAGCAT
and
(SEQ ID NO: 36)
ACGGTCCGCCAGAAGA;
RPLP0:
(SEQ ID NO: 37)
CCACGCTGCTGAACATGCT,
(SEQ ID NO: 38)
TCGAACACCTGCTGGATGAC
and
(SEQ ID NO: 39)
TCTCCCCCTTCTCCTTTG
and
SDHA:
(SEQ ID NO: 40)
AGGAATCAATGCTGCTCTGGG,
(SEQ ID NO: 41)
GTCGGAGCCCTTCACGGT
and
(SEQ ID NO: 42)
CCACCTCCAGTTGTCC.

In various embodiments, primers and/or probes with sequences that are capable of hybridizing with each of above-referenced genes and references genes are used to measure the gene expression level of each gene. In various other embodiments, the metastatic clear cell renal cell carcinoma gene and reference gene expression level is determined by RNAseq, microarray and/or nanostring.

In some embodiments, the metastatic clear cell renal cell carcinoma gene and reference gene expression level are determined using quantitative polymerase chain reaction (qPCR), RNAseq, microarray and/or nanostring, using specific primer sequences and/or a probe sequences found in Table 2.

### Subjects

In various embodiments, the subject is human. In various embodiments, the subject is suspected to have renal cell carcinoma. In various embodiments, the subject is diagnosed to have metastatic clear cell renal cell carcinoma. In various embodiments, the subject is treated for renal cell carcinoma.

### Biological Samples

In various embodiments, the steps involved in the current invention comprise obtaining either through surgical biopsy or surgical resection, a sample from the subject. Alternatively, a sample can be obtained through primary patient derived cell lines, or archived patient samples in the form of FFPE (Formalin fixed, paraffin embedded) samples, or fresh frozen samples.

Patient sample is then used to extract nucleic acid (Ribonucleic acid (RNA), Deoxyribonucleic acid (DNA)) or protein, using standard protocols well-known in the art.

### Sample Preparation and Gene Expression Detection

Nucleic acid or protein samples derived from cancerous and non-cancerous cells of a subject that can be used in the methods of the invention to determine the genetic signature of a cancer can be prepared by means well known in the art. For example, surgical procedures or needle biopsy aspiration can be used to collect cancerous samples from a subject. In some embodiments, it is important to enrich and/or purify the cancerous tissue and/or cell samples from the non-cancerous tissue and/or cell samples. In other embodiments, the cancerous tissue and/or cell samples can then be microdissected to reduce the amount of normal tissue contamination prior to extraction of genomic nucleic acid or pre-RNA for use in the methods of the invention. Such enrichment and/or purification can be accomplished according to methods well-known in the art, such as needle microdissection, laser microdissection, fluorescence activated cell sorting, and immunological cell sorting.

Analysis of the nucleic acid and/or protein from an individual may be performed using any of various techniques. In various embodiments, assaying gene expression levels for CRYL1, CEP55, PCNA, TRAF2, HGF, CDK1, HSD17B10, USP6NL, ACTB, RPL13A, GUS, RPLP0, HPRT1, and SDHA comprises, northern blot, reverse transcription PCR, real-time PCR, serial analysis of gene expression (SAGE), DNA microarray, tiling array, RNA-Seq, or a combination thereof. In various other embodiments, one or more of the 416 additional clear cell renal cell carcinoma genes are also assessed.

In various embodiments, methods and systems to detect protein expression include but are not limited to ELISA, immunohistochemistry, western blot, flow cytometry, fluorescence in situ hybridization (FISH), radioimmuno assays, and affinity purification.

As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The analysis of gene expression levels may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and sybr green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

In certain embodiments of the methods of the invention, the nucleic acid from a subject is amplified using primer pairs. In various embodiments, the nucleic acid of a subject is amplified using sets of primer pairs specific to CRYL1, CEP55, PCNA, TRAF2, HGF, CDK1, HSD17B10, USP6NL and to sets of primer pairs specific to the housekeeping genes, ACTB, RPL13A, GUS, RPLP0, HPRT1, SDHA.

A DNA sample suitable for hybridization can be obtained, e.g., by polymerase chain reaction (PCR) amplification of genomic DNA, fragments of genomic DNA, fragments of genomic DNA ligated to adaptor sequences or cloned sequences. Computer programs that are well known in the art can be used in the design of primers with the desired specificity and optimal amplification properties, such as Oligo version 5.0 (National Biosciences). PCR methods are well known in the art, and are described, for example, in Innis et al., eds., 1990, PCR Protocols: A Guide to Methods And Applications, Academic Press Inc., San Diego, Calif. It will be apparent to one skilled in the art that controlled robotic systems are useful for isolating and amplifying nucleic acids and can be used.

### Hybridization

The nucleic acid samples derived from a subject used in the methods of the invention can be hybridized to arrays comprising probes (e.g., oligonucleotide probes) in order to identify CRYL1, CEP55, PCNA, TRAF2, HGF, CDK1, HSD17B10 and USP6NL and in instances wherein a housekeeping gene expression is also to be assessed, comprising probes in order to identify the housekeeping genes discussed above. In particular embodiments, the probes used in the methods of the invention comprise an array of probes that can be tiled on a DNA chip (e.g., SNP oligonucleotide probes). Hybridization and wash conditions used in the methods of the invention are chosen so that the nucleic acid samples to be analyzed by the invention specifically bind or specifically hybridize to the complementary oligonucleotide sequences of the array, preferably to a specific array site, wherein its complementary DNA is located. In some embodiments, the complementary DNA can be completely matched or mismatched to some degree as used, for example, in Affymetrix oligonucleotide arrays. The single-stranded synthetic oligodeoxyribonucleic acid DNA probes of an array may need to be denatured prior to contact with the nucleic acid samples from a subject, e.g., to remove hairpins or dimers which form due to self-complementary sequences.

Optimal hybridization conditions will depend on the length of the probes and type of nucleic acid samples from a subject. General parameters for specific (i.e., stringent) hybridization conditions for nucleic acids are described in Sambrook and Russel, Molecular Cloning: A Laboratory Manual 4^{th} ed., Cold Spring Harbor Laboratory Press (Cold Spring Harbor, N.Y. 2012); Ausubel et al., eds., 1989, Current Protocols in Molecules Biology, Vol. 1, Green Publishing Associates, Inc., John Wiley & Sons, Inc., New York, at pp. 2.10.1-2.10.16. Exemplary useful hybridization conditions are provided in, e.g., Tijessen, 1993, Hybridization with Nucleic Acid Probes, Elsevier Science Publishers B. V. and Kricka, 1992, Nonisotopic DNA Probe Techniques, Academic Press, San Diego, Calif.

### Oligonucleotide Nucleic Acid Arrays

In some embodiments of the methods of the present invention, DNA arrays can be used to determine the expression levels of genes, by measuring the level of hybridization of the nucleic acid sequence to oligonucleotide probes that comprise complementary sequences. Various formats of DNA arrays that employ oligonucleotide “probes,” (i.e., nucleic acid molecules having defined sequences) are well known to those of skill in the art. Typically, a set of nucleic acid probes, each of which has a defined sequence, is immobilized on a solid support in such a manner that each different probe is immobilized to a predetermined region. In certain embodiments, the set of probes forms an array of positionally-addressable binding (e.g., hybridization) sites on a support. Each of such binding sites comprises a plurality of oligonucleotide molecules of a probe bound to the predetermined region on the support. More specifically, each probe of the array is preferably located at a known, predetermined position on the solid support such that the identity (i.e., the sequence) of each probe can be determined from its position on the array (i.e., on the support or surface). Microarrays can be made in a number of ways, of which several are described herein. However produced, microarrays share certain characteristics, they are reproducible, allowing multiple copies of a given array to be produced and easily compared with each other.

In some embodiments, the microarrays are made from materials that are stable under binding (e.g., nucleic acid hybridization) conditions. The microarrays are preferably small, e.g., between about 1 cm² and 25 cm², preferably about 1 to 3 cm². However, both larger and smaller arrays are also contemplated and may be preferable, e.g., for simultaneously evaluating a very large number of different probes. Oligonucleotide probes can be synthesized directly on a support to form the array. The probes can be attached to a solid support or surface, which may be made, e.g., from glass, plastic (e.g., polypropylene, nylon), polyacrylamide, nitrocellulose, gel, or other porous or nonporous material. The set of immobilized probes or the array of immobilized probes is contacted with a sample containing labeled nucleic acid species so that nucleic acids having sequences complementary to an immobilized probe hybridize or bind to the probe. After separation of, e.g., by washing off, any unbound material, the bound, labeled sequences are detected and measured. The measurement is typically conducted with computer assistance. DNA array technologies have made it possible to determine the expression level of CRYL1, CEP55, PCNA, TRAF2, HGF, CDK1, HSD17B10, USP6NL, and/or one or more of the 416 additional clear cell renal cell carcinoma genes and/or housekeeping genes, as mentioned above.

In certain embodiments, high-density oligonucleotide arrays are used in the methods of the invention. These arrays containing thousands of oligonucleotides complementary to defined sequences, at defined locations on a surface can be synthesized in situ on the surface by, for example, photolithographic techniques (see, e.g., Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; 5,510,270; 5,445,934; 5,744,305; and 6,040,138). Methods for generating arrays using inkjet technology for in situ oligonucleotide synthesis are also known in the art (see, e.g., Blanchard, International Patent Publication WO 98/41531, published Sep. 24, 1998; Blanchard et al., 1996, Biosensors And Bioelectronics 11:687-690; Blanchard, 1998, in Synthetic DNA Arrays in Genetic Engineering, Vol. 20, J. K. Setlow, Ed., Plenum Press, New York at pages 111-123). Another method for attaching the nucleic acids to a surface is by printing on glass plates, as is described generally by Schena et al. (1995, Science 270:467-470). Other methods for making microarrays, e.g., by masking (Maskos and Southern, 1992, Nucl. Acids. Res. 20:1679-1684), may also be used. When these methods are used, oligonucleotides (e.g., 15 to 60-mers) of known sequence are synthesized directly on a surface such as a derivatized glass slide. The array produced can be redundant, with several oligonucleotide molecules corresponding to each informative locus of interest (e.g., SNPs, RFLPs, STRs, etc.).

One exemplary means for generating the oligonucleotide probes of the DNA array is by synthesis of synthetic polynucleotides or oligonucleotides, e.g., using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 15 and about 600 bases in length, more typically between about 20 and about 100 bases, most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., 1993, Nature 363:566-568; U.S. Pat. No. 5,539,083). In alternative embodiments, the hybridization sites (i.e., the probes) are made from plasmid or phage clones of regions of genomic DNA corresponding to SNPs or the complement thereof. The size of the oligonucleotide probes used in the methods of the invention can be at least 10, 20, 25, 30, 35, 40, 45, or 50 nucleotides in length. It is well known in the art that although hybridization is selective for complementary sequences, other sequences which are not perfectly complementary may also hybridize to a given probe at some level. Thus, multiple oligonucleotide probes with slight variations can be used, to optimize hybridization of samples. To further optimize hybridization, hybridization stringency condition, e.g., the hybridization temperature and the salt concentrations, may be altered by methods that are well known in the art.

In various embodiments, the high-density oligonucleotide arrays used in the methods of the invention comprise oligonucleotides corresponding to CRYL1, CEP55, PCNA, TRAF2, HGF, CDK1, HSD17B10, USP6NL, and/or one or more of the 416 additional clear cell renal cell carcinoma genes and/or housekeeping genes, as mentioned above. The oligonucleotide probes may comprise DNA or DNA “mimics” (e.g., derivatives and analogues) corresponding to a portion of each informative locus of interest (e.g., SNPs, RFLPs, STRs, etc.) in a subject's genome. The oligonucleotide probes can be modified at the base moiety, at the sugar moiety, or at the phosphate backbone. Exemplary DNA mimics include, e.g., phosphorothioates. For each SNP locus, a plurality of different oligonucleotides may be used that are complementary to the sequences of sample nucleic acids. For example, for a single informative locus of interest (e.g., SNPs, RFLPs, STRs, etc.) about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more different oligonucleotides can be used. Each of the oligonucleotides for a particular informative locus of interest may have a slight variation in perfect matches, mismatches, and flanking sequence around the SNP. In certain embodiments, the probes are generated such that the probes for a particular informative locus of interest comprise overlapping and/or successive overlapping sequences which span or are tiled across a genomic region containing the target site, where all the probes contain the target site. By way of example, overlapping probe sequences can be tiled at steps of a predetermined base interval, e. g. at steps of 1, 2, 3, 4, 5, 6, 7, 8, 9, or 10 bases intervals. In certain embodiments, the assays can be performed using arrays suitable for use with molecular inversion probe protocols such as described by Wang et al. (2007) Genome Biol. 8, R246. For oligonucleotide probes targeted at nucleic acid species of closely resembled (i.e., homologous) sequences, “cross-hybridization” among similar probes can significantly contaminate and confuse the results of hybridization measurements. Cross-hybridization is a particularly significant concern in the detection of SNPs since the sequence to be detected (i.e., the particular SNP) must be distinguished from other sequences that differ by only a single nucleotide. Cross-hybridization can be minimized by regulating either the hybridization stringency condition and/or during post-hybridization washings. Highly stringent conditions allow detection of allelic variants of a nucleotide sequence, e.g., about 1 mismatch per 10-30 nucleotides. There is no single hybridization or washing condition which is optimal for all different nucleic acid sequences, these conditions can be identical to those suggested by the manufacturer or can be adjusted by one of skill in the art. In some embodiments, the probes used in the methods of the invention are immobilized (i.e., tiled) on a glass slide called a chip. For example, a DNA microarray can comprises a chip on which oligonucleotides (purified single-stranded DNA sequences in solution) have been robotically printed in an (approximately) rectangular array with each spot on the array corresponds to a single DNA sample which encodes an oligonucleotide. In summary the process comprises, flooding the DNA microarray chip with a labeled sample under conditions suitable for hybridization to occur between the slide sequences and the labeled sample, then the array is washed and dried, and the array is scanned with a laser microscope to detect hybridization. In certain embodiments there are at least 250, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 50,000, 60,000, 70,000, 80,000, 90,000, 100,000 or more or any range in between, of CRYL1, CEP55, PCNA, TRAF2, HGF, CDK1, HSD17B10, USP6NL, and/or one or more of the 416 additional clear cell renal cell carcinoma genes and/or the housekeeping genes for which probes appear on the array (with match/mismatch probes for a single locus of interest or probes tiled across a single locus of interest counting as one locus of interest). The maximum number of CRYL1, CEP55, PCNA, TRAF2, HGF, CDK1, HSD17B10, USP6NL, and/or one or more of the 416 additional clear cell renal cell carcinoma genes and/or the housekeeping genes being probed per array is determined by the size of the genome and genetic diversity of the subjects species. DNA chips are well known in the art and can be purchased in pre-5 fabricated form with sequences specific to particular species. In other embodiments, SNPs and/or DNA copy number can be detected and quantitated using sequencing methods, such as “next-generation sequencing methods” as described further above.

### Signal Detection

In some embodiments, nucleic acid samples derived from a subject are hybridized to the binding sites of an array described herein. In certain embodiments, nucleic acid samples derived from each of the two sample types of a subject (i.e., cancerous and non-cancerous) are hybridized to separate, though identical, arrays. In certain embodiments, nucleic acid samples derived from one of the two sample types of a subject (i.e., cancerous and non-cancerous) is hybridized to such an array, then following signal detection the chip is washed to remove the first labeled sample and reused to hybridize the remaining sample. In other embodiments, the array is not reused more than once. In certain embodiments, the nucleic acid samples derived from each of the two sample types of a subject (i.e., cancerous and non-cancerous) are differently labeled so that they can be distinguished. When the two samples are mixed and hybridized to the same array, the relative intensity of signal from each sample is determined for each site on the array, and any relative difference in abundance of CRYL1, CEP55, PCNA, TRAF2, HGF, CDK1, HSD17B10, USP6NL and/or the 416 additional clear cell renal cell carcinoma genes. Signals can be recorded and, in some embodiments, analyzed by computer. In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluorophores may be made. For any particular hybridization site on the array, a ratio of the emission of the two fluorophores can be calculated, which may help in eliminating cross hybridization signals to more accurately determining whether a particular SNP locus is heterozygous or homozygous.

### Labeling

In some embodiments, the protein, polypeptide, nucleic acid, fragments thereof, or fragments thereof ligated to adaptor regions used in the methods of the invention are detectably labeled. For example, the detectable label can be a fluorescent label, e.g., by incorporation of nucleotide analogues. Other labels suitable for use in the present invention include, but are not limited to, biotin, iminobiotin, antigens, cofactors, dinitrophenol, lipoic acid, olefinic compounds, detectable polypeptides, electron rich molecules, enzymes capable of generating a detectable signal by action upon a substrate, and radioactive isotopes.

Radioactive isotopes include that can be used in conjunction with the methods of the invention, but are not limited to, 32P and 14C. Fluorescent molecules suitable for the present invention include, but are not limited to, fluorescein and its derivatives, rhodamine and its derivatives, texas red, 5′carboxy-fluorescein (“FAM”), 2′, 7′-dimethoxy-4′, 5′-dichloro-6-carboxy-fluorescein (“JOE”), N, N, N′, N′-tetramethyl-6-carboxy-rhodamine (“TAMRA”), 6-carboxy-X-rhodamine (“ROX”), HEX, TET, IRD40, and IRD41.

Fluorescent molecules which are suitable for use according to the invention further include: cyamine dyes, including but not limited to Cy2, Cy3, Cy3.5, CY5, Cy5.5, Cy7 and FLUORX; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable for the present invention include, but are not limited to, ferritin, hemocyanin and colloidal gold.

Two-color fluorescence labeling and detection schemes may also be used (Shena et al., 1995, Science 270:467-470). Use of two or more labels can be useful in detecting variations due to minor differences in experimental conditions (e.g., hybridization conditions). In some embodiments of the invention, at least 5, 10, 20, or 100 dyes of different colors can be used for labeling. Such labeling would also permit analysis of multiple samples simultaneously which is encompassed by the invention.

The labeled nucleic acid samples, fragments thereof, or fragments thereof ligated to adaptor regions that can be used in the methods of the invention are contacted to a plurality of oligonucleotide probes under conditions that allow sample nucleic acids having sequences complementary to the probes to hybridize thereto. Depending on the type of label used, the hybridization signals can be detected using methods well known to those of skill in the art including, but not limited to, X-Ray film, phosphor imager, or CCD camera. When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al. (1996) Genome Res. 6, 639-645). In a preferred embodiment, the arrays are scanned with a laser fluorescence scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al. (1996) Genome Res. 6, 639-645. Alternatively, a fiber-optic bundle can be used such as that described by Ferguson et al. (1996) Nat. Biotech. 14, 1681-1684. The resulting signals can then be analyzed to determine the expression of CRYL1, CEP55, PCNA, TRAF2, HGF, CDK1, HSD17B10, USP6NL, and/or one or more of the 416 additional clear cell renal cell carcinoma genes and/or the reference genes, using computer software.

In other embodiments, where genomic DNA of a subject is fragmented using restriction endonucleases and amplified prior to analysis, the amplification can comprise cloning regions of genomic DNA of the subject. In such methods, amplification of the DNA regions is achieved through the cloning process. For example, expression vectors can be engineered to express large quantities of particular fragments of genomic DNA of the subject (Sambrook and Russel, Molecular Cloning: A Laboratory Manual 4^{th} ed., Cold Spring Harbor Laboratory Press (Cold Spring Harbor, N.Y. 2012)).

In yet other embodiments, where the DNA of a subject is fragmented using restriction endonucleases and amplified prior to analysis, the amplification comprises expressing a nucleic acid encoding a gene, or a gene and flanking genomic regions of nucleic acids, from the subject. RNA (pre-messenger RNA) that comprises the entire transcript including introns is then isolated and used in the methods of the invention to analyze and provide a genetic signature of a cancer. In certain embodiments, no amplification is required. In such embodiments, the genomic DNA, or pre-RNA, of a subject may be fragmented using restriction endonucleases or other methods. The resulting fragments may be hybridized to SNP probes. Typically, greater quantities of DNA are needed to be isolated in comparison to the quantity of DNA or pre-mRNA needed where fragments are amplified. For example, where the nucleic acid of a subject is not amplified, a DNA sample of a subject for use in hybridization may be about 400 ng, 500 ng, 600 ng, 700 ng, 800 ng, 900 ng, or 1000 ng of DNA or greater. Alternatively, in other embodiments, methods are used that require very small amounts of nucleic acids for analysis, such as less than 400 ng, 300 ng, 200 ng, 100 ng, 90 ng, 85 ng, 80 ng, 75 ng, 70 ng, 65 ng, 60 ng, 55 ng, 50 ng, or less, such as is used for molecular inversion probe (MIP) assays. These techniques are particularly useful for analyzing clinical samples, such as paraffin embedded formalin-fixed material or small core needle biopsies, characterized as being readily available but generally having reduced DNA quality (e.g., small, fragmented DNA) and/or not providing large amounts of nucleic acids.

### CRYL1, CEP55, PCNA, TRAF2, HGF, CDK1, HSD17B10, USP6NL and the 416 Additional Clear Cell Renal Cell Carcinoma Genes Expression Analysis

Analysis of CRYL1 expression levels can be determined by quantitative PCR which can provide a quantified expression level within the sample for the gene. The quantified gene expression levels are normalized to reference genes and converted into a coefficient, which is used to determine overall survival in the subject. For example, if the coefficient is negative with a low gene expression level or if the coefficient is positive with a high gene expression level, then the subject is determined to have a good overall survival and if the coefficient is negative with a high gene expression level or if the coefficient is positive with a low gene expression level, then the subject is determined to have a poor overall survival. In various embodiments, the coefficient is calculated from the slope of a multi-variant regression model. In some embodiments, the coefficient is the slope of the multi-variant regression model. In other embodiments, the coefficients come from a model that includes the eight genes and the MSKCC adverse clinical risk factors. In various other embodiments, the coefficients are calculated from a model that includes the MSKCC adverse clinical risk factors or the eight genes. In various embodiments, a multi-variant analysis is used to obtain the time dependent area-under-the-curve. In various other embodiments, a hazard ratio is calculated. In certain embodiments, the hazard ratio is calculated using the equation HR=exp(coefficient). In various embodiments, a risk score is calculated for each patient from the regression coefficients. In some embodiments, the risk score is calculated using the equation RS=Σ(ln(HR)×normalized gene expression). In other embodiments, the risk score is then calibrated for survival. In other embodiments, the genes are plotted over time in a Kaplan-Meier curve. The analysis for CEP55, PCNA, TRAF2, HGF, CDK1, HSD17B10, USP6NL and/or one or more of the 416 additional clear cell renal cell carcinoma genes expression can be similarly performed.

In various embodiments, the subject is stratified into a low, intermediate and high risk group for metastatic clear cell renal cell carcinoma from the risk score and overall survival. In other embodiments, a subject in a low risk group has a good overall survival, a subject in an intermediate risk group has an intermediate overall survival and the subject in a high risk group has a poor overall survival.

In various embodiments, the analysis of CRYL1, CEP55, PCNA, TRAF2, HGF, CDK1, HSD17B10, USP6NL and/or one or more of the 416 additional clear cell renal cell carcinoma genes expression levels are performed via the methods described herein.

### Algorithms for Analyzing CRYL1, CEP55, PCNA, TRAF2, HGF, CDK1, HSD17B10, USP6NL

Once the expression levels have been determined, the resulting data can be analyzed using various algorithms. In certain embodiments, the algorithms for determining the expression of CRYL1, CEP55, PCNA, TRAF2, HGF, CDK1, HSD17B10, USP6NL, and/or one or more of the 416 additional clear cell renal cell carcinoma genes and the reference genes is based on well-known methods.

### Kits

The present invention is also directed to a kit to determine overall survival in a subject with metastatic renal cell carcinoma. The kit is useful for practicing the inventive method of determining overall survival. The kit is an assemblage of materials or components, including at least one of the inventive compositions. Thus, in some embodiments the kit contains a composition including primers and probes for metastatic renal cell carcinoma genes and reference genes, as described above.

The exact nature of the components configured in the inventive kit depends on its intended purpose. For example, some embodiments are configured for the purpose of determining gene expression levels. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to determine overall survival. Optionally, the kit also contains other useful components, such as, primers, diluents, buffers, pipetting or measuring tools or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing primers and probes for metastatic renal cell carcinoma genes and reference genes. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Patient Population

Patients eligible for CALGB 90206 had metastatic or unresectable RCC with a clear cell histology, Karnofsky performance status ≧70%, and adequate organ function. Prior chemotherapy for metastatic disease was not permitted. A stratified random block design was used with the stratification factors of nephrectomy and number of adverse risk factors as defined by the Motzer criteria (Motzer et al., 2002, J. Clin. Oncol. 20 (1), 289-296). All details of the clinical trial are published elsewhere (Rini et al., 2008, J. Clin. Oncol. 26(33), 5422-5428 and Rini et al., 2010, J. Clin. Oncol. 28 (13), 2137-2143). Each participant signed an IRB-approved, protocol-specific informed consent in accordance with federal and institutional guidelines. Data collection and statistical analyses were conducted by the Alliance Statistics and Data Center.

### RNA Extraction

Using tumors received as part of CALGB90206, H&E stains were made of samples received by CALGB and reviewed by a genitourinary (GU) pathologist (JS) who annotated the outline of the tumor on a digital image, which was used to macrodissection the tumor for RNA extraction. All assay work was performed at Cedars Sinai (HK). Our method for RNA extraction from FFPE renal tumors has been previously described (Glenn et al., 2010, J. Biomol Screen (15)1:80-85). Briefly, RNA was extracted from six 10-μm sections when archival blocks were available. Some participating sites chose to send unstained slides, and three 5-μm sections were used.

Tumor sections were placed in 2.0-mL RNase-free Eppendorf tubes. Sections were treated twice with 1 mL xylene for 5 min at 55° C. while rocking. The sections were washed twice with 100% ethanol. RNA was extracted from the paraffin samples using the MasterPure™ RNA Purification Kit (Epicentre Biotechnologies, Madison, Wis., USA). In an attempt to further increase RNA yield, FFPE samples were treated with 200 ug Proteinase K for 3 hr at 55° C. RNA was then treated with 20 units DNase I (Ambion, Austin, Tex., USA), for 30 min and checked for residual genomic DNA by TaqMan RT-PCR targeting ACTB. If there was measurable DNA after 34 PCR cycles using 50 ng input RNA, the samples were treated with 20 units DNase I for an additional 15 min, and the assay for residual DNA was repeated. The final RNA concentration (A260:0.025) and purity (A260:A280 ratio) was measured using a NanoDrop ND-2000 spectrophotometer (NanoDrop Technologies, Wilmington, Del., USA).

### Reverse Transcription (RT)

Reverse transcription (RT) was performed using the High Capacity cDNA Reverse Transcription Kit (Life Technologies, Grand Island, N.Y.) following the manufacturer's recommendation. Each 10 μl RT reaction contained 150 ng of total RNA (75 ng or 37.5 ng was used for cases with lower RNA yield), 1 μl of 10×RT buffer, 0.5 μl of 25×dNTP mixture, 1 μl of 10× random reverse primers, 1 μl of 10× gene-specific reverse primers (1 μM) and 0.5 μl of MultiScribe RT (50 U/μl). The 10 μl reactions were incubated in a Life Technologies Thermocycler for 10 min at 25° C., 2 hours at 37° C., 5 min at 85° C. and then held at 4° C. 10× pooled gene-specific reverse primers (1 μM) were prepared by combining equal volumes of each 500 μM reverse primer (primers for all candidate genes were pooled). The same primers were used for gene-specific RT, preamplification and qPCR. The candidate genes were identified from a literature search for prognostic and predictive gene expressions determined from microarrays and tissue microarrays. Key genes involved in pathways known to be important in RCC were also included.

The preamplification was performed using TaqMan® PreAmp Master Mix Kit (Life Technologies, Grand Island, N.Y.) as previously described (Li et al., 2013, Bioanalysis 5(13):1623-33). Each 5 μl of preamplification reaction included 2.5 μl of 2× TaqMan® PreAmp Master Mix, 1.25 μl of 0.09× pooled Taqman assay mix and 1.25 μl of cDNA. The reactions were incubated in an Applied Biosystems Thermocycler for 10 min at 95° C. followed by 13-15 cycles (depending on starting RNA) of 95° C. for 15 seconds and 60° C. for 4 min and then held at 4° C. Pooled Taqman assays (0.09×) were prepared by combining equal volumes of each 20× Taqman assay (needed for PCR on the Openarray®, 218 assays for each set). Each cDNA was preamplified on two sets of 218 pooled assays. Preamplified cDNA products were diluted 10 times with 1×TE buffer for storage and PCR.

### Real-Time PCR on OpenArray Platform

Two sets of TaqMan® OpenArray® Real-Time PCR Plates (Life Technologies, Grand Island, N.Y.) were made using custom-designed primers and probes. Our strategy for designing TaqMan® assays (Li et al., 2013, Bioanalysis 5(13):1623-33). Briefly, gene sequences were downloaded from GenBank (http://www.ncbi.nlm.nih.gov/genbank/). Repeats and low complexity sequences, and SNPs were masked. The resulting sequences were sent to Life Technologies for custom design of primers and probes using their proprietary software. Each PCR target was blasted to avoid amplification of unintended targets. When multiple isoforms existed, targets were selected in regions common to all isoforms. All primers were designed to generate amplicons less than 100 base pairs (Table 2). Each OpenArray® Real-Time PCR Plate contains 218 TaqMan® assays. Diluted preamplified cDNA (10 μl) was mixed with 10 μl of TaqMan® OpenArray® Real-Time PCR Master Mix (Life Technologies, Grand Island, N.Y.). The mixed cDNA samples were dispensed into an OpenArray® 384-well Sample Plate (Life Technologies, Grand Island, N.Y.) with each sample placed into 4 wells at 5 μl per well. cDNA samples were then dispensed into OpenArray® Real-Time PCR Plates using OpenArray® AccuFill™ System (Life Technologies, Grand Island, N.Y.). The real-time PCR reactions were incubated in an OpenArray® NT Cycler system for 2 min at 50° C., 10 min at 95° C. followed by 40 cycles of 95° C. for 15 seconds and 60° C. for 1 min and then held at 4° C.

TABLE 2
List of Primers and Probes for
Prognostic Markers and Reference Genes.

| | Forward | SEQ | Reverse | SEQ | | SEQ |
|---|---|---|---|---|---|---|
| | Primer | ID | Primer | ID | Probe | ID |
| | Sequence | NO | Sequence | NO | Sequence | NO |
| Prognostic | | | | | | |
| Marker | | | | | | |
| CDK1 | ACCTATGG | 1 | ACCCCTTC | 2 | CATGGCTA | 3 |
| | AGTTGTGT | | CTCTTCAC | | CCACTTGA | |
| | ATAAGGGT | | TTTCTAGT | | CC | |
| | AGAC | | | | | |
| CEP55 | CTCCAAAC | 4 | ACACGAGC | 5 | CTCCAGAG | 6 |
| | TGCTTCAA | | CACTGCTG | | CATCTTTC | |
| | CTCATCAA | | ATTTT | | | |
| | T | | | | | |
| CRYL1 | CGTTGGCA | 7 | GGAAGCCT | 8 | ATGGCCCA | 9 |
| | GTGGAGTC | | CCACTGGC | | GCTTCGCC | |
| | ATTG | | AAA | | | |
| HGF | CATTCACT | 10 | TTTCACTC | 11 | AACAATGC | 12 |
| | TGCAAGGC | | CACTTGAC | | CTCTGGTT | |
| | TTTTGTTT | | ATGCTATT | | CCC | |
| | T | | GA | | | |
| HSD17B10 | CCAAGCCA | 13 | GCTGTTTG | 14 | CCCAGCCG | 15 |
| | AGAAGTTA | | CACATCCT | | ACGTGACC | |
| | GGAAACAA | | TCTCAGA | | | |
| | C | | | | | |
| PCNA | TGAACCTC | 16 | CGTTATCT | 17 | CCGGCGCA | 18 |
| | ACCAGTAT | | TCGGCCCT | | TTTTAGT | |
| | GTCCAAAA | | TAGTGTAA | | | |
| | T | | T | | | |
| TRAF2 | GGAAGCGC | 19 | CCGTACCT | 20 | ATACCCGC | 21 |
| | CAGGAAGC | | GCTGGTGT | | CATCTTCT | |
| | T | | AGAAG | | | |
| USP6NL | GAGGAGCT | 22 | GCATTTTC | 23 | AAGCACCT | 24 |
| | CCCAGATC | | AGCCATTT | | GGAAATTG | |
| | ATAATGTG | | GGTAGTTC | | | |
| | | | T | | | |
| Reference | | | | | | |
| Gene | | | | | | |
| ACTB | CCAGCTCA | 25 | ATGCCGGA | 26 | TCGCCGCG | 27 |
| | CCATGGAT | | GCCGTTGT | | CTCGTC | |
| | GATG | | C | | | |
| GUSB | CTCATTTG | 28 | CCGAGTGA | 29 | TCACCGAC | 30 |
| | GAATTTTG | | AGATCCCC | | GAGAGTGC | |
| | CCGATT | | TTTTTA | | | |
| HPRT1 | ATGGACAG | 31 | GCACACAG | 32 | CCTCCCAT | 33 |
| | GACTGAAC | | AGGGCTAC | | CTCCTTCA | |
| | GTCTTG | | AATGT | | TCA | |
| RPL13A | ACCAACCC | 34 | TTGGTTTT | 35 | ACGGTCCG | 36 |
| | TTCCCGAG | | GTGGGGCA | | CCAGAAGA | |
| | GC | | GCAT | | | |
| RPLP0 | CCACGCTG | 37 | TCGAACAC | 38 | TCTCCCCC | 39 |
| | CTGAACAT | | CTGCTGGA | | TTCTCCTT | |
| | GCT | | TGAC | | TG | |
| SDHA | AGGAATCA | 40 | GTCGGAGC | 41 | CCACCTCC | 42 |
| | ATGCTGCT | | CCTTCACG | | AGTTGTCC | |
| | CTGGG | | GT | | | |

Post-acquisition data processing generated fluorescence amplification for each assay, from which cycle threshold (CT) were computed and used for further data analysis. Each gene was normalized using 6 reference genes, which were measured in quadruplicate (Glenn et al., 2007, Biotechniques 43(5), 639-40, 42-3, 47). Each PCR plate had a control cDNA and ACTB amplification was always with 0.5 CT of the expected value. When over half the candidate genes failed detection in any given sample, the sample was disqualified and not used for analysis. Tumor expression data were generated for 430 candidate genes identified from a literature search. CT levels were normalized with six housekeeping genes. Expression levels that were too low to detect were imputed to 30, which corresponded to a single copy of a gene in the assay chamber (Li et al., 2013, Bioanalysis 5(13):1623-33).

### Statistical Analysis

The primary end point used for this analysis was overall survival (OS), defined as the time from randomization to date of death of any cause. The date of data cutoff for the clinical trial was Mar. 24, 2009 and median follow up among surviving patients was 46.2 months. The dataset was randomly divided at 2:1 ratio into training (n=221) and testing (n=103) sets to develop a multigene prognostic signature. Training and testing samples were normalized together prior to random allocation. To adjust for any lingering batch effects, we calculated gene means and standard deviations within each batch, then centered and scaled samples to have within-batch gene means 0 and standard deviations 1. The comparative CT method was used to analyze the data (also referred to as ΔΔC_{T}) (Schmittgen and Livak, 2008, Nat. Protoc. 3(6), 1101-1108).

There were 12 individuals with two samples from different regions of the tumor that were used to assess heterogeneity. We used the median of the 12 standard deviations as a measure of stability and chose a cut-point of 0.78. K-means clustering algorithm was utilized to identify the threshold for the stable genes.

### Model Building

Several steps were used for model building to help prioritize genes for the multivariable model of OS. First, univariate proportional hazards models were fit in the training set to test for the prognostic importance of the 424 genes in predicting overall survival. Twenty-one genes had q-value (false discovery rate)<0.05 in the univariate scans and were selected for the multivariable model. In the second step, the least absolute shrinkage and selection operator (LASSO) penalty was used to identify important genes for the multivariable model (Tibshirani, 1997, Stat. Med. 16 (4), 385-395). The main advantage of using penalized methods is that they produce sparse regression coefficients, and the selection of important prognostic factors does not depend on statistical significance. The regularization parameter was chosen to minimize the Schwarz Information Criterion. The 95% CI for the LASSO was derived by adopting the perturbation method proposed by Minnier and extending their work to the Cox's regression (Minnier et al., 2011, Int. J. Urol. 16 (5), 465-471 and Lin and Halabi, 2015, Communications in Statistics: Theory and Methods, in press). In the final step, all possible multivariable models of eight genes from 21 potentially important genes were fit to the training data (203,490 multivariable models). The top 100 models were ranked by the concordance index and the highest time-dependent AUC (tdAUC) and the final model was chosen accordingly. A risk score was calculated for each patient using the estimated regression coefficients from the training set.

### Validation

The parameter estimates from the locked final model were applied to the testing set and a risk score was computed for every patient. The performance of the final model was assessed by computing the tdAUC. In addition, the tdAUC was computed for the eight gene model and for the model containing only the Memorial Sloan Kettering Cancer Center (MSKCC) adverse clinical risk factors. The 95% CI for the tdAUC was computed using the bootstrapped approach. The final model was validated with the risk score as a continuous variable. Tertiles based on the training set were identified and applied to the risk score in the testing set. Patients were grouped into low, intermediate or high-risk groups. The final model was validated by one of the authors (SH) who did not have access to the training set. The Kaplan-Meier product-limit method was used to estimate the overall survival distribution by the different risk groups and the log-rank statistic was used to test if the three-risk groups have different survival outcomes. All statistical analyses for model development and validation were performed using the R package.

### Example 2

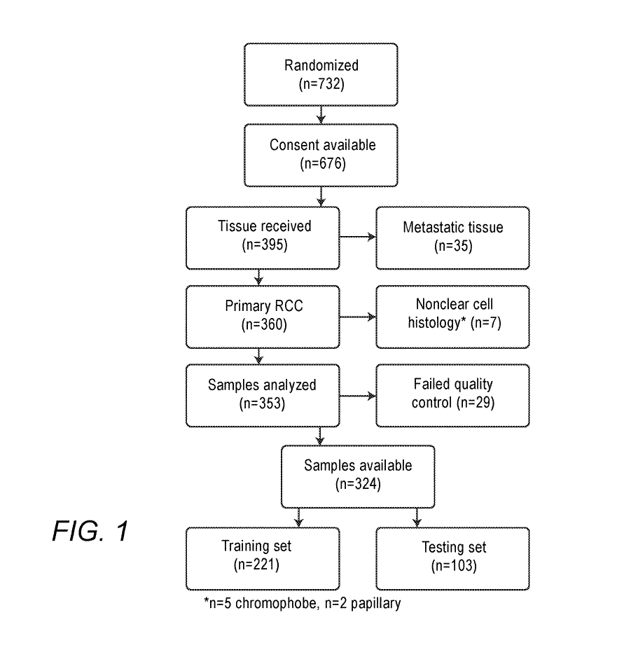

CALGB90206 enrolled 732 patients in the United States and Canada between October 2003 and July 2005. The primary outcomes from the parent trial have been previously reported (Rini et al., 2008, J. Clin. Oncol. 26(33), 5422-5428 and Rini et al., 2010, J. Clin. Oncol. 28 (13), 2137-2143). After enrollment 10 patients did not meet eligibility, 26 patients met exclusion criteria, and 16 patients refused to participate. FIG. 1 presents the REMARK diagram. Consents for use of tissue for correlative studies were obtained form 92% (676/732) of patients (FIG. 1). The eligibility for the parent trial required primary tumor tissue to be available. However, tissue submission was not required for patients to start treatment. Paraffin-embedded tumor blocks or unstained slides were received for 395 patients. All tissues were H&E stained and centrally reviewed by a single GU pathologist (J.S.). Cases were excluded where the tissue was from a metastatic site or the primary tumor was not a clear cell RCC. A total of 353 cases were analyzed by qPCR and 29 cases failed quality control. The available cases were randomly split 2:1 into training and testing sets. The final analysis was based on 324 patients with available specimens.

Patient demographics are summarized in Table 3 and various subgroups were compared for the training and test sets. The baseline clinical characteristics for patients in the training and testing sets were comparable and similar to that of the entire cohort enrolled on CALGB 90206 with a few exceptions. Given that most tumor tissue came from cytoreductive nephrectomies, the percent of patients having nephrectomy was higher in study subjects (99% vs 73%). Patients for whom we received clear cell RCC from the primary tumor were the subject of this study. This group was compared to all other patients enrolled in the parent trial. Patient demographics for the 29 patients that failed qPCR quality control are presented.

TABLE 3
Patient Demographics

| | | | | Failed | | | | | | |
|---|---|---|---|---|---|---|---|---|---|---|
| | RCC | | RCC | quality | Training | | Testing | | | |
| | not available | | available | control | Set | | Set | | Total | |
| | (n = 379) | | (n = 353) | (n = 29) | (n = 221) | | (n = 103) | | (n = 732) | |
| Gender (%) | | | | | | | | | | |
| Male | 267 | (70) | 68% | 66% | 152 | (69) | 69 | (67) | 505 | (69) |
| Female | 112 | (30) | 32% | 34% | 69 | (31) | 34 | (33) | 227 | (31) |
| Median Age, Years | 62 | | 61 | 59 | 61 | | 63 | | 62 | |
| (25^{th}, 75^{th} percentile) | (56-70) | | (55-70) | (53-70) | (55-69) | | (56-71) | | (55-70) | |
| Nephrectomy (%) | 276 | (73) | 97% | 83% | 218 | (99) | 102 | (99) | 620 | (85) |
| ECOG performance | | | | | | | | | | |
| status (%) | | | | | | | | | | |
| 0 | 122 | (33) | 39% | 31% | 92 | (42) | 36 | (35) | 259 | (36) |
| 1 | 225 | (60) | 54% | 66% | 112 | (51) | 58 | (56) | 414 | (57) |
| 2 | 27 | (7) | 7% | 3% | 14 | (6) | 8 | (8) | 50 | (7) |
| Unknown | 5 | (0) | 1% | 0% | 3 | (1) | 1 | (1) | 9 | (1) |
| Common Sites of | | | | | | | | | | |
| Metastases* (%) | | | | | | | | | | |
| Lung | 248 | (66) | 73% | 66% | 165 | (75) | 75 | (73) | 507 | (69) |
| Lymph node | 130 | (34) | 37% | 38% | 89 | (40) | 29 | (28) | 259 | (35) |
| Bone | 32 | (33) | 25% | 31% | 59 | (27) | 22 | (21) | 213 | (29) |
| Liver | 95 | (25) | 15% | 17% | 32 | (14) | 15 | (15) | 147 | (20) |
| Number of Risk | | | | | | | | | | |
| Factor** (%) | | | | | | | | | | |
| 0 (favorable) | 101 | (27) | 26% | 31% | 61 | (28) | 21 | (20) | 192 | (26) |
| 1-2 (intermediate) | 231 | (61) | 66% | 55% | 144 | (65) | 74 | (72) | 456 | (64) |
| >=3 (poor) | 47 | (12) | 8% | 14% | 16 | (7) | 8 | (8) | 75 | (10) |

| | | | | | | |
|---|---|---|---|---|---|---|
| Treatment | | | | | | |
| IFNα | 48% | 51% | 52% | 54% | 46% | 50% |
| bevacizumab + IFNα | 52% | 49% | 48% | 46% | 54% | 50% |

**MSKCC, adverse clinical risk factors
*Not mutally exclusive

Candidate genes were identified from a literature search focused on prognostic and predictive biomarkers for clear cell RCC discovered from gene microarray and large tissue microarray studies. TaqMan PCR assays were custom made for 424 candidate genes. Using the training set (n=221), all 424 genes were evaluated in the proportional hazards model in predicting OS. The top 25 prognostic genes are presented in Table 4. These genes with q-value<0.05 were considered as candidate genes in the multivariable analysis. The hazard ratios (HR) for normalized ΔΔC_{T} values are provided. Lower ΔΔC_{T}'s corresponds to higher expression levels, therefore HRs<1 indicate higher expression level and decreased risk of death. Univariate analysis was also performed for the 424 genes using the entire cohort of 324 subjects.

TABLE 4
Top 25 Prognostic Genes in Training Set*

| | HR** | CI** | p-value | q-value |
|---|---|---|---|---|
| MCM2 | 0.7 | (0.60-0.82) | <0.0001 | 0.00297 |
| CCNB1 | 0.74 | (0.64-0.86) | <0.0001 | 0.01036 |
| TOP2A | 0.75 | (0.64-0.87) | 0.00015 | 0.01036 |
| NPM3 | 0.74 | (0.63-0.86) | 0.00016 | 0.01036 |
| CEP55 | 0.75 | (0.64-0.87) | 0.00025 | 0.01282 |
| FSCN1 | 0.76 | (0.65-0.88) | 0.00036 | 0.0152 |
| KIAA0101 | 0.76 | (0.65-0.89) | 0.00052 | 0.01912 |
| CRYL1 | 1.3 | (1.11-1.53) | 0.00088 | 0.02507 |
| CDK1 | 0.77 | (0.65-0.90) | 0.00098 | 0.02507 |
| KIF23 | 0.78 | (0.67-0.90) | 0.00105 | 0.02507 |
| L1CAM | 0.78 | (0.68-0.91) | 0.00108 | 0.02507 |
| TRAF2 | 0.78 | (0.67-0.91) | 0.00127 | 0.02582 |
| ANLN | 0.77 | (0.65-0.90) | 0.00131 | 0.02582 |
| KLK1 | 0.77 | (0.66-0.91) | 0.0017 | 0.02968 |
| HGF | 0.78 | (0.66-0.91) | 0.00174 | 0.02968 |
| USP6NL | 0.79 | (0.68-0.92) | 0.00265 | 0.04073 |
| PCNA | 0.8 | (0.69-0.93) | 0.00286 | 0.04073 |
| MELK | 0.77 | (0.65-0.92) | 0.0029 | 0.04073 |
| PRC1 | 0.78 | (0.66-0.92) | 0.00312 | 0.04073 |
| POLR2B | 0.8 | (0.69-0.93) | 0.00322 | 0.04073 |
| HSD17B10 | 0.8 | (0.69-0.93) | 0.00334 | 0.04073 |
| ITGB1 | 0.81 | (0.70-0.94) | 0.00479 | 0.05571 |
| NME1 | 0.82 | (0.71-0.94) | 0.00552 | 0.06138 |
| TTK | 0.82 | (0.71-0.95) | 0.00702 | 0.07278 |
| MKI67 | 0.81 | (0.70-0.95) | 0.00711 | 0.07278 |

*Genes in our final prognostic model are in bold
**HR, hazard ratio; CI, 95% confidence interval

### Multivariable Model

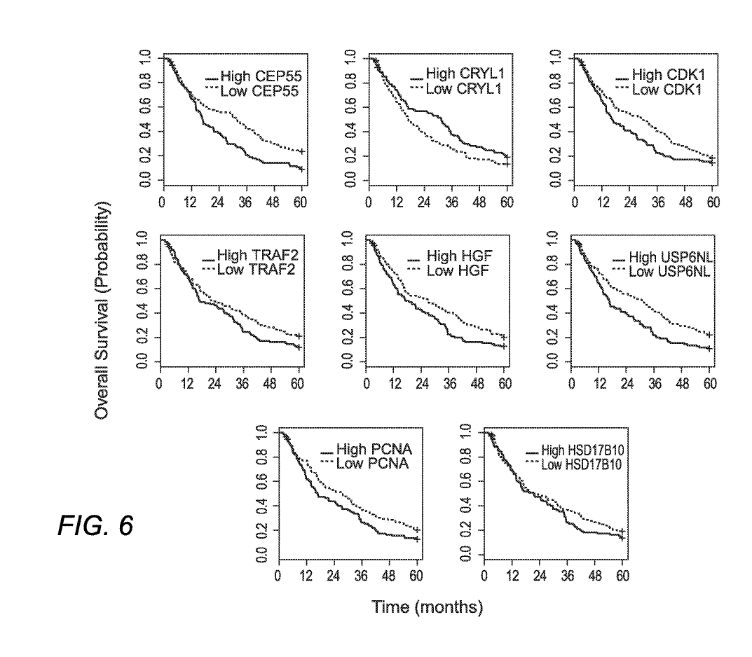

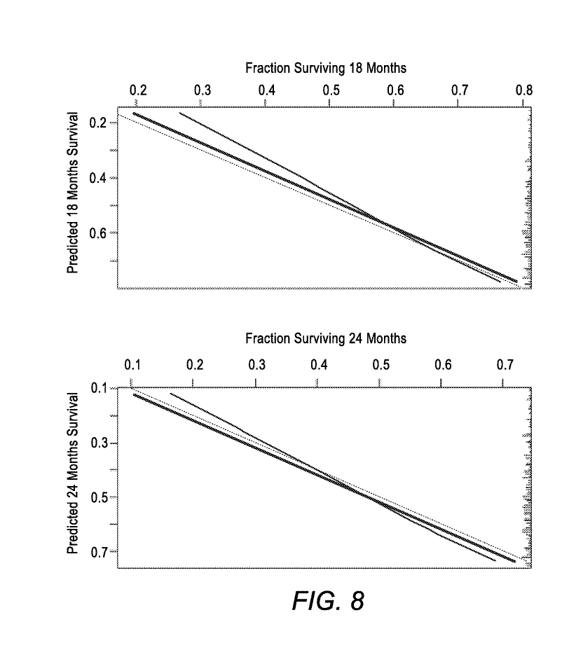

Using LASSO 8 genes were identified. Therefore, 8 genes were determined as the optimal size for the final model and all possible models of eight genes out of 21 significant genes were fit. For illustrative purposes, the Kaplan-Meier plots are provided for each of the eight genes that were included in the final model. The genes are dichotomized by the observed medians into high and low expression groups (FIG. 6). In the parent clinical trial, the number of MSKCC clinical risk factors was used as a stratification factor in the randomization. Therefore, MSKCC clinical risk factors were included in the final multivariable model (Table 5A). In the training set, the tdAUC for the final 8-gene model with the MSKCC risk factors was 0.71 (95% CI=0.59-0.73). For CRYL1, PCNA and CDK1, decreased expression levels were associated with worse OS; however, for TRAF2, USP6NL, CEP55, HGF and HSD17B10, the inverse association was observed. The final model was assessed for calibration (internal validation). The predicted probabilities at 18 (median OS in the clinical trial)-, and 24-months from the model were close to the observed probability of survival (FIG. 8).

The final model included MSKCC risk factors and the combination of 8 markers that produced the highest time dependent area-under-the-cure (tdAUC) in a multivariate analysis. MSKCC alone only had modest prognostic ability in our population with a tdAUC of 0.611 (Table 5B). Our final multi-marker signature model, which was developed and locked prior to application to the testing set, has a tdAUC of 0.71 when applied to our randomly selected test subset (Table 5B). The final model was locked and coefficients estimated from the training set were used to compute a risk score (RS) for each patient in the test set. The tdAUC for the final model applied to the test set is presented in Table 5B.

TABLE 5A
Prognostic Model for Overall Survival

| | Coefficients | HR* | 95% CI* | p-value |
|---|---|---|---|---|
| CRYL1 | 0.356 | 1.428 | (1.188, 1.716) | 0.0001 |
| TRAF2 | −0.215 | 0.806 | (0.688,0.945) | 0.0079 |
| USP6NL | −0.090 | 0.914 | (0.751, 1.111) | 0.0101 |
| CEP55 | −0.258 | 0.772 | (0.634, 0.940) | 0.0246 |
| HGF | −0.086 | 0.918 | (0.761, 1.107) | 0.1818 |
| PCNA | 0.155 | 1.167 | (0.930, 1.464) | 0.3657 |
| CDK1 | 0.089 | 1.093 | (0.870, 1.372) | 0.3688 |
| HSD17B10 | −0.232 | 0.793 | (0.648, 0.971) | 0.4449 |
| 1-2 RF** vs. 0 | 0.276 | 1.317 | (0.939, 1.849) | 0.111 |
| >3 RF vs. 0 | 0.954 | 2.596 | (1.467, 4.594) | 0.001 |

*HR, hazard ratio; CI, 95% confidence interval
**Rf, MSKCC Adverse Clinial Risk Factors

TABLE 5B
Performance of Prognostic Models in the Test Set

| | tdAUC | p-value |
|---|---|---|
| 8 genes + RF | 0.723 | <0.001 |
| 8 genes | 0.688 | <0.001 |
| RF | 0.611 | 0.008 |

tdAUC: time dependent area under the curve;
RF, MSKCC Adverse Clinial Risk Factors

### Testing Set

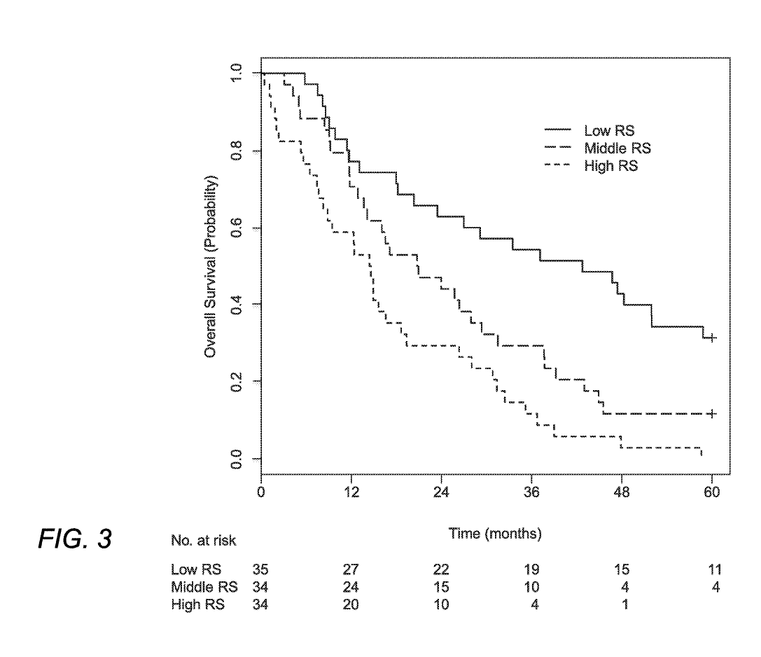

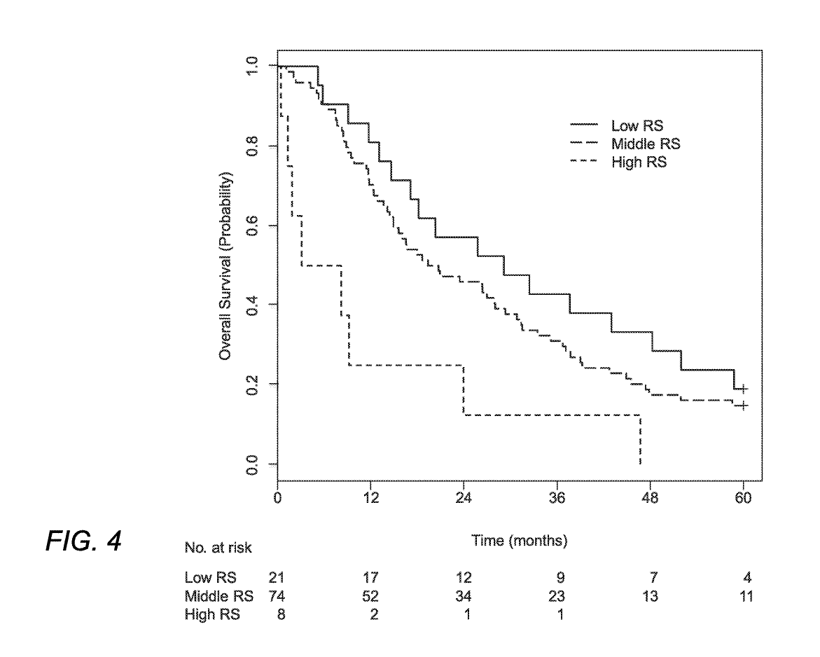

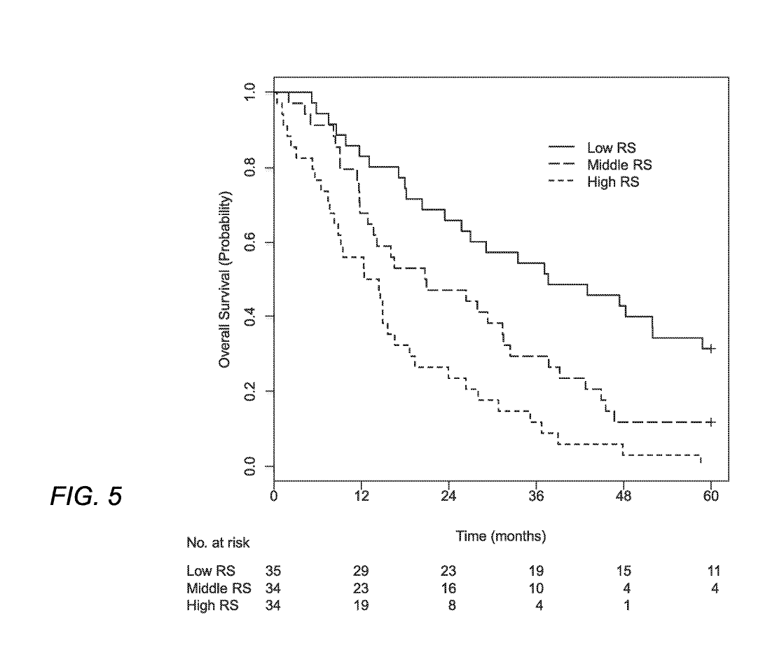

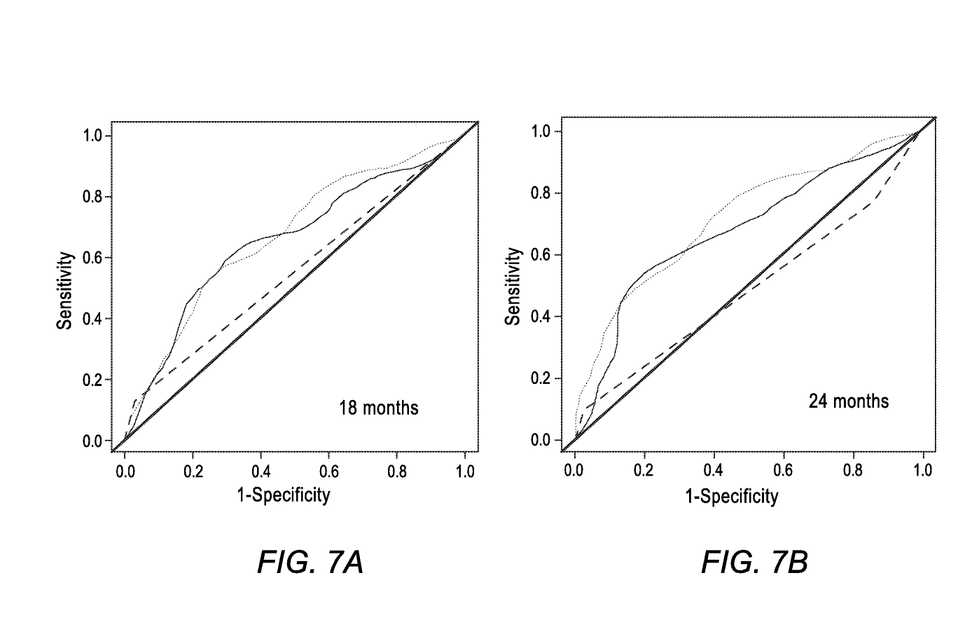

Using the final model, RS was calculated for each patient in the test set. The risk score was highly predictive of OS with a tdAUC=0.72 (95% CI=0.66-0.78). The testing set was divided into equal thirds to generate cutoffs for low, intermediate, and high risk groups. Median OSs were 38 (95% CI=26—not reached), 21 (95% CI=14-32) and 13 (95% CI=9-19) months, respectively (p<0.001, FIG. 5). For comparison, the 8-gene model without the MSKCC clinical risk factors was similarly applied to the testing set (FIG. 3) and had a tdAUC of 0.69 (95% CI=0.62-0.72). FIG. 4 presents the Kaplan-Meier survival curves for risk groups based on number of MSKCC clinical risk factors. It is noteworthy that the tdAUC for this model was only 0.61 (95% CI=0.54-0.69). FIG. 7 shows the AUC at 18 (FIG. 7A) and 24 months (FIG. 7B) for the three models. It is clear that the final model based on the 8 genes and the MSKCC clinical risk factors is superior to the model with MSKCC clinical risk factors alone.

### Stability Analysis

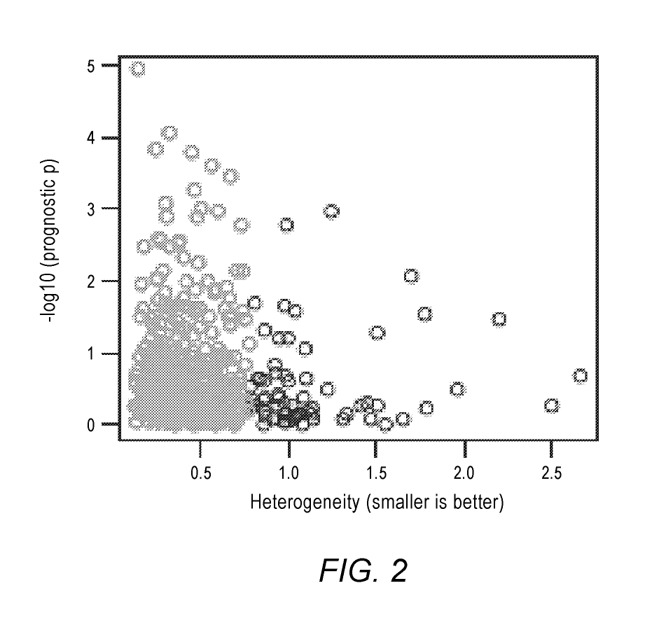

Mutations in individual renal tumors are highly heterogeneous. Therefore, to ensure the stability of our gene signature, the expression of the 424 candidate genes were measured from two random sites using 12 primary clear cell RCCs from patient with metastatic disease. For each gene we calculated standard deviations for each RCC. The stability measure for each gene was the median of the 12 standard deviations. K-means clustering (K=2) was used to determine a threshold (0.78) for dividing the genes into stable and unstable genes based on the stability measure (FIG. 2). Of the 424 genes, 83 genes were considered unstable. All 8 genes in our final model were confirmed to be stable. In a post-hoc analysis, we repeated our model building approach using stable genes only, and achieved a test AUC of 0.72, just below our locked model. This is in part due to the fact that the most significant genes in univariate analysis tended to be stable genes.

### Example 3

Approximately one-third of patients newly diagnosed with RCC have metastatic disease, and after treatment for localized RCC, 25-50% of patients will suffer metastatic recurrence. The survival for individual patients can vary widely. Patients can be stratified into risk groups based on readily available clinical parameters such as performance status, serum lactate dehydrogenase, hemoglobin, serum calcium, and length of time between initial diagnosis and treatment (Motzer et al., 2002, J. Clin. Oncol. 20 (1), 289-296). The number of MSKCC adverse clinical risk factors was used to stratify the randomization for the parent clinical trial of this study, CALGB 90206. Unfortunately, MSKCC adverse clinical risk factors only had modest prognostic ability in our population with a tdAUC of 0.61, demonstrating the need for developing predictive markers with higher precision.

The inventors developed a molecular prognostic signature based on 8 genes and MSKCC adverse clinical risk factors and tested the molecular prognostic signature using tissue from a phase III trial to predict OS in patients with metastatic clear cell RCC. This study used the multimarker prognostic signature from a large multicenter phase III, randomized clinical trial in RCC in which eligibility was clearly defined and outcomes rigorously recorded in a diverse range of patients. Importantly, the prognostic signature was obtained using formalin-fixed, paraffin-embedded tumors, which are routinely collected and stored in all pathology departments. For clear cell RCC, tumor tissue is routinely available from cytoreductive nephrectomy or diagnostic biopsy. To the best of our knowledge, there are no prior reports of a multimarker molecular signature developed from a multicenter, phase III clinical trial of RCC.

CALGB 90206 randomized patients with newly diagnosed clear cell RCC to IFN or IFN plus bevacizumab. The primary endpoint was overall survival, and secondary endpoints were progression free survival and safety. The majority (85%) of patients underwent a cytoreductive nephrectomy, and 90% had favorable or intermediate prognosis based on number of MSKCC adverse clinical risk factors. At the interim analysis, the median PFS was 5.2 months in the IFN group and 8.5 months in the IFN plus bevacizumab group (p<0.0001). However, no statistically significant difference in OS was observed between the two groups. The median OS was 17.4 months in the IFN group and 18.3 months in the combination arm (Rini et al., 2010, J. Clin. Oncol. 28 (13), 2137-2143). Furthermore, subset analysis failed to identify any clinical variable associated with treatment response. Therefore, no clinical variable other than MSKCC adverse clinical risk factors were included in our final model.

An important limitation of prior biomarkers studies is that they included all stages of RCC, limiting their applicability to patients with metastatic disease. However, many of these studies used an unbiased, genome-wide approach to discovery of prognostic markers, and provided a wealth of candidate gene expression markers for us to evaluate. An increased understanding of pathways and mechanisms driving clear cell RCC provided additional candidate markers. In univariate analysis, we found that 21 of the candidate biomarkers were significant predictors of OS using q<0.05 (Table 4). These results are validation of prior discovery studies of prognostic markers.

Our multivariable analysis identified an 8-gene model of OS. Following VHL inactivation, PBRM1 is the second major gene in ccRCC, with truncating mutations in 41% of cases. Genes in pathways deregulated following PBRM1 knockdown in RCC cell lines were included as candidate genes in this study. In our final prognostic model, 4 genes (CRYL1, HSD17B10, CEP55 and HGF) were PBRM1 related genes; 3 (CRYL1, HSD17B10 and CEP55) were also differentially expressed when comparing ccRCC to normal kidney. HGF, which binds the proto-oncogene c-MET, has been linked to invasiveness and VHL inactivation in ccRCC. Both TRAF2 and USP6NL were previously identified as prognostic genes is microarray-based studies of RCC. PCNA was included as a candidate gene because it is a classic marker of proliferation and has been previously associated with RCC prognosis. CDK1, a cell cycle regulator, was included as a candidate gene because it was previously reported to predict response to antiangiogenic and epidermal growth factor targeted therapy in RCC. When generating our prognostic signature, genes were favored that provided independent and non-redundant prognostic information. Therefore, it is not surprising that our 8 genes have been associated with a wide range of functions important to cancer progression such as proliferation (CEP55, PCNA, CDK1), apoptosis (TRAF2), metabolism (CRYL1, HSD17B10) and invasion (HGF) (http://www.ncbi.nlm.nih.gov/gene, 2015).

The genetic heterogeneity of RCC is well documented. However, the clonal evolutionary tree has a common “trunk” that links all genomic mutations. In addition, there are common histologic features that pathologists use to classify renal tissue as RCC. Therefore, it is reasonable to expect that there are markers, particularly expression markers that directly reflect the phenotype of RCC. To generate a signature that was less sensitive to sampling artifacts produced by tumor heterogeneity, we performed a separate analysis using untreated primary tumors from metastatic clear cell RCC patients that were sampled in two different areas. Genes with heterogeneous expression within individual patients were excluded from consideration in our multimarker models.

There are several strengths of the present analysis. First, the trial had a large number of tissue specimens available. Second, the data were from a randomized multi-institutional phase III trial. The parent trial clearly defines the patient cohort for which the signature can be applied. Furthermore, patient treatment and follow-up have been rigorously recorded, with oversight from a highly developed coordinating center.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.

1. A method of determining overall survival in a subject with metastatic clear cell renal cell carcinoma, comprising:
obtaining a biological sample from the subject with metastatic clear cell renal cell carcinoma;
assaying the biological sample to determine an expression level for a metastatic clear cell renal cell carcinoma gene and a reference gene,
wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof;
normalizing the metastatic clear cell renal cell carcinoma gene expression level to the reference gene expression level;
calculating a coefficient or using a calculated coefficient for each metastatic clear cell renal cell carcinoma gene; and
determining that the subject has a good overall survival if the coefficient is negative with a low gene expression level or if the coefficient is positive with a high gene expression level and determining that the subject has poor overall survival if the coefficient is negative with a high gene expression level or if the coefficient is positive with a low gene expression level.

2. The method of claim 1, wherein the metastatic clear cell renal cell carcinoma gene and reference gene expression level is determined using quantitative polymerase chain reaction (qPCR), using a specific primer sequence and/or a probe sequence.

3. The method of claim 2, wherein the primer sequence or probe sequence used for the metastatic clear cell renal cell carcinoma gene and the reference gene is:
CDK1:
(SEQ ID NO: 1)
ACCTATGGAGTTGTGTATAAGGGTAGAC,
(SEQ ID NO: 2)
ACCCCTTCCTCTTCACTTTCTAGT
and
(SEQ ID NO: 3)
CATGGCTACCACTTGACC;
CEP55:
(SEQ ID NO: 4)
CTCCAAACTGCTTCAACTCATCAAT,
(SEQ ID NO: 5)
ACACGAGCCACTGCTGATTTT
and
(SEQ ID NO: 6)
CTCCAGAGCATCTTTC;
CRYL1:
(SEQ ID NO: 7)
CGTTGGCAGTGGAGTCATTG,
(SEQ ID NO: 8)
GGAAGCCTCCACTGGCAAA
and
(SEQ ID NO: 9)
ATGGCCCAGCTTCGCC;
HGF:
(SEQ ID NO: 10)
CATTCACTTGCAAGGCTTTTGTTTT,
(SEQ ID NO: 11)
TTTCACTCCACTTGACATGCTATTGA
and
(SEQ ID NO: 12)
AACAATGCCTCTGGTTCCC;
HSD17B10:
(SEQ ID NO: 13)
CCAAGCCAAGAAGTTAGGAAACAAC,
(SEQ ID NO: 14)
GCTGTTTGCACATCCTTCTCAGA
and
(SEQ ID NO: 15)
CCCAGCCGACGTGACC;
PCNA:
(SEQ ID NO: 16)
TGAACCTCACCAGTATGTCCAAAAT,
(SEQ ID NO: 17)
CGTTATCTTCGGCCCTTAGTGTAAT
and
(SEQ ID NO: 18)
CCGGCGCATTTTAGT;
TRAF2:
(SEQ ID NO: 19)
GGAAGCGCCAGGAAGCT,
(SEQ ID NO: 20)
CCGTACCTGCTGGTGTAGAAG
and
(SEQ ID NO: 21)
ATACCCGCCATCTTCT;
USP6NL:
(SEQ ID NO: 22)
GAGGAGCTCCCAGATCATAATGTG,
(SEQ ID NO: 23)
GCATTTTCAGCCATTTGGTAGTTCT
and
(SEQ ID NO: 24)
AAGCACCTGGAAATTG;
ACTB:
(SEQ ID NO: 25)
CCAGCTCACCATGGATGATG,
(SEQ ID NO: 26)
ATGCCGGAGCCGTTGTC
and
(SEQ ID NO: 27)
TCGCCGCGCTCGTC;
GUSB:
(SEQ ID NO: 28)
CTCATTTGGAATTTTGCCGATT,
(SEQ ID NO: 29)
CCGAGTGAAGATCCCCTTTTTA
and
(SEQ ID NO: 30)
TCACCGACGAGAGTGC;
HPRT1:
(SEQ ID NO: 31)
ATGGACAGGACTGAACGTCTTG,
(SEQ ID NO: 32)
GCACACAGAGGGCTACAATGT
and
(SEQ ID NO: 33)
CCTCCCATCTCCTTCATCA;
RPL13A:
(SEQ ID NO: 34)
ACCAACCCTTCCCGAGGC,
(SEQ ID NO: 35)
TTGGTTTTGTGGGGCAGCAT
and
(SEQ ID NO: 36)
ACGGTCCGCCAGAAGA;
RPLP0:
(SEQ ID NO: 37)
CCACGCTGCTGAACATGCT,
(SEQ ID NO: 38)
TCGAACACCTGCTGGATGAC
and
(SEQ ID NO: 39)
TCTCCCCCTTCTCCTTTG
and
SDHA:
(SEQ ID NO: 40)
AGGAATCAATGCTGCTCTGGG,
(SEQ ID NO: 41)
GTCGGAGCCCTTCACGGT
and
(SEQ ID NO: 42)
CCACCTCCAGTTGTCC.

4. The method of claim 1, wherein the calculated coefficient for the metastatic clear cell renal cell carcinoma gene CDK1 is 0.089, CEP55 is −0.258, CRYL1 is 0.356, HGF is −0.086, HSD17B10 is −0.232, PCNA is 0.155, TRAF2 is −0.215 and USP6NL is −0.090.

5. The method of claim 1, wherein the metastatic clear cell renal cell carcinoma gene and reference gene expression level is determined by RNAseq, microarray and/or nanostring.

6. The method of claim 1, further comprising using one or more MSKCC adverse clinical risk factors selected from the group consisting of Karnofsky performance status, serum lactate dehydrogenase, serum hemoglobin, serum calcium, length of time between initial diagnosis and treatment, and combinations thereof, to aid in determining overall survival.

7. The method of claim 6, wherein a coefficient is calculated for the one or more MSKCC adverse clinical risk factors.

8. The method of claim 7, wherein the MSKCC adverse clinical risk factor coefficient for 1 and/or 2 MSKCC adverse clinical risk factors is 0.276 or the coefficient for 3 or more MSKCC adverse clinical risk factors is 0.954.

9. The method of claim 1, wherein the coefficient is calculated from the slope of a multi-variant regression model.

10. The method of claim 1, further comprising assaying the biological sample to determine an expression level for one or more of the 416 additional clear cell renal cell carcinoma genes.

11. A method of determining overall survival in a subject with metastatic clear cell renal cell carcinoma, comprising:
obtaining a biological sample from the subject with metastatic clear cell renal cell carcinoma;
assaying the biological sample to determine an expression level for a metastatic clear cell renal cell carcinoma gene and a reference gene,
wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof;
normalizing the metastatic clear cell renal cell carcinoma gene expression level to the reference gene expression level;
calculating a hazard ratio or using a calculated hazard ratio for each metastatic clear cell renal cell carcinoma gene;
calculating a risk score from the hazard ratio; and
stratifying the subject into a low, intermediate and high risk group for metastatic clear cell renal cell carcinoma from the risk score; wherein a subject in a low risk group has a good overall survival, a subject in an intermediate risk group has an intermediate overall survival and the subject in a high risk group has a poor overall survival.

12-14. (canceled)

15. A process of patient risk stratification, comprising:
obtaining a biological sample from a subject with metastatic clear cell renal cell carcinoma;
assaying the biological sample to determine an expression level for a metastatic clear cell renal cell carcinoma gene and a reference gene,
wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof;
normalizing the metastatic clear cell renal cell carcinoma gene expression level to the reference gene expression level;
calculating a coefficient or using a calculated coefficient for each metastatic clear cell renal cell carcinoma gene;
calculating a risk score from the coefficient; and
stratifying the subject into risk groups for metastatic clear cell renal cell carcinoma from the risk score.

16-24. (canceled)

25. A method of selecting a therapy and/or treatment for metastatic renal cell carcinoma, comprising:
obtaining a biological sample from a subject with metastatic clear cell renal cell carcinoma;
assaying the biological sample to determine an expression level for a metastatic clear cell renal cell carcinoma gene and a reference gene,
wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof;
normalizing the metastatic clear cell renal cell carcinoma gene expression level to the reference gene expression level;
calculating a coefficient or using a calculated coefficient for each metastatic clear cell renal cell carcinoma gene;
calculating a risk score from the coefficient;
stratifying the subject into a low, intermediate and high risk group for metastatic clear cell renal cell carcinoma from the risk score; wherein a subject in a low risk group has a good overall survival, a subject in an intermediate risk group has an intermediate overall survival and the subject in a high risk group has a poor overall survival; and
selecting a first therapy for a subject in the low, intermediate and high risk group, selecting a second therapy for a subject in the low or intermediate risk group, and selecting a third therapy, or a combination of the first, second and third therapy for a subject in a high risk group.

26-33. (canceled)

34. The method of claim 25, wherein patient counseling is given to a subject that has been stratified into a low, intermediate or high risk group.

35. The method of claim 25, wherein the first therapy is selected from the group consisting of surgical resection, radical or partial nephrectomy, active surveillance, palliative radiation therapy, metastasectomy and/or bisphonates.

36. The method of claim 25, wherein the second therapy is a targeted therapy drug or immunotherapy.

37. The method of claim 36, wherein the targeted therapy drug is selected from the group consisting of VEGF inhibitors or mTOR inhibitors.

38. The method of claim 37, wherein the VEGF inhibitors are selected from the group consisting of Sunitinib, Pazopanib, Bevacizumab, Sorafenib, Axitinib, and combinations thereof.

39. The method of claim 37, wherein the mTOR inhibitors are selected from the group consisting of Temsirolimus, Everolimus, and combinations thereof.

40. The method of claim 36, wherein the immunotherapy is selected from the group consisting of high-dose Interleukin-2, low-dose Interleukin-2, Interferon-alpha 2a or combinations thereof.

41. The method of claim 25, wherein the third therapy is thermal ablation, a combination of the first and second therapy, and combinations thereof.

42. The method of claim 41, wherein thermal ablation comprises cryoablation and radiofrequency ablation.

43. A method of selecting a metastatic clear cell renal cell carcinoma subject for a clinical trial, comprising:
obtaining a biological sample from a subject with metastatic clear cell renal cell carcinoma;
assaying the biological sample to determine an expression level for a metastatic clear cell renal cell carcinoma gene and a reference gene,
wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof;
normalizing the metastatic clear cell renal cell carcinoma gene expression level to the reference gene expression level;
calculating a coefficient or using a calculated coefficient for each metastatic clear cell renal cell carcinoma gene;
calculating a risk score from the coefficients;
stratifying the subject into a low, intermediate and high risk group for metastatic clear cell renal cell carcinoma from the risk score; wherein a subject in a low risk group has a good overall survival, a subject in an intermediate risk group has an intermediate overall survival and the subject in a high risk group has a poor overall survival; and
selecting the subject for a clinical trial if the subject falls within the appropriate risk group for the clinical trial, wherein a subject in a low risk group is selected for a low risk group clinical trial, a subject in an intermediate risk group is selected for an intermediate risk group clinical trial and a subject in a high risk group is selected for a high risk group clinical trial.

44-51. (canceled)

52. A method of determining overall survival in a subject with metastatic clear cell renal cell carcinoma, comprising:
obtaining a biological sample from a subject with metastatic clear cell renal cell carcinoma;
assaying the biological sample to determine an expression level for a metastatic clear cell renal cell carcinoma gene and a reference gene,
wherein the metastatic clear cell renal cell carcinoma gene is selected from the group consisting of CRYL1, CEP55, PCNA, TRAF2, HGF, CDK1, HSD17B10, USP6NL and combinations thereof, and the reference gene is selected from the group consisting of ACTB, RPL13A, GUS, RPLP0, HPRT1, SDHA and combinations thereof;
normalizing the metastatic clear cell renal cell carcinoma gene expression level to the reference gene expression level; and
determining that the subject has a good overall survival if there is a increased expression level of CRYL1, PCNA, CDK1, or a combination thereof, or if there is a decreased expression level of TRAF2, USP6NL, CEP55, HGF, HSD17B10 or a combination thereof, and determining that the subject has poor overall survival if there is a decreased expression level of CRYL1, PCNA, CDK1, or a combination thereof, or if there is an increased expression level of TRAF2, USP6NL, CEP55, HGF, HSD17B10 or a combination thereof.

53-54. (canceled)


SEQUENCE LISTING (42 sequences)

SEQ ID NO: 1 - DNA, 28 bp, Artificial Sequence
  Human
acctatggagttgtgtataagggtagac

SEQ ID NO: 2 - DNA, 24 bp, Artificial Sequence
  Human
accccttcctcttcactttctagt

SEQ ID NO: 3 - DNA, 18 bp, Artificial Sequence
  Human
catggctaccacttgacc

SEQ ID NO: 4 - DNA, 25 bp, Artificial Sequence
  Human
ctccaaactgcttcaactcatcaat

SEQ ID NO: 5 - DNA, 21 bp, Artificial Sequence
  Human
acacgagccactgctgatttt

SEQ ID NO: 6 - DNA, 16 bp, Artificial Sequence
  Human
ctccagagcatctttc

SEQ ID NO: 7 - DNA, 20 bp, Artificial Sequence
  Human
cgttggcagtggagtcattg

SEQ ID NO: 8 - DNA, 19 bp, Artificial Sequence
  Human
ggaagcctccactggcaaa

SEQ ID NO: 9 - DNA, 16 bp, Artificial Sequence
  Human
atggcccagcttcgcc

SEQ ID NO: 10 - DNA, 25 bp, Artificial Sequence
  Human
cattcacttgcaaggcttttgtttt

SEQ ID NO: 11 - DNA, 26 bp, Artificial Sequence
  Human
tttcactccacttgacatgctattga

SEQ ID NO: 12 - DNA, 19 bp, Artificial Sequence
  Human
aacaatgcctctggttccc

SEQ ID NO: 13 - DNA, 25 bp, Artificial Sequence
  Human
ccaagccaagaagttaggaaacaac

SEQ ID NO: 14 - DNA, 23 bp, Artificial Sequence
  Human
gctgtttgcacatccttctcaga

SEQ ID NO: 15 - DNA, 16 bp, Artificial Sequence
  Human
cccagccgacgtgacc

SEQ ID NO: 16 - DNA, 25 bp, Artificial Sequence
  Human
tgaacctcaccagtatgtccaaaat

SEQ ID NO: 17 - DNA, 25 bp, Artificial Sequence
  Human
cgttatcttcggcccttagtgtaat

SEQ ID NO: 18 - DNA, 15 bp, Artificial Sequence
  Human
ccggcgcattttagt

SEQ ID NO: 19 - DNA, 17 bp, Artificial Sequence
  Human
ggaagcgccaggaagct

SEQ ID NO: 20 - DNA, 21 bp, Artificial Sequence
  Human
ccgtacctgctggtgtagaag

SEQ ID NO: 21 - DNA, 16 bp, Artificial Sequence
  Human
atacccgccatcttct

SEQ ID NO: 22 - DNA, 24 bp, Artificial Sequence
  Human
gaggagctcccagatcataatgtg

SEQ ID NO: 23 - DNA, 25 bp, Artificial Sequence
  Human
gcattttcagccatttggtagttct

SEQ ID NO: 24 - DNA, 16 bp, Artificial Sequence
  Human
aagcacctggaaattg

SEQ ID NO: 25 - DNA, 20 bp, Artificial Sequence
  Human
ccagctcaccatggatgatg

SEQ ID NO: 26 - DNA, 17 bp, Artificial Sequence
  Human
atgccggagccgttgtc

SEQ ID NO: 27 - DNA, 14 bp, Artificial Sequence
  Human
tcgccgcgctcgtc

SEQ ID NO: 28 - DNA, 22 bp, Artificial Sequence
  Human
ctcatttggaattttgccgatt

SEQ ID NO: 29 - DNA, 22 bp, Artificial Sequence
  Human
ccgagtgaagatccccttttta

SEQ ID NO: 30 - DNA, 16 bp, Artificial Sequence
  Human
tcaccgacgagagtgc

SEQ ID NO: 31 - DNA, 22 bp, Artificial Sequence
  Human
atggacaggactgaacgtcttg

SEQ ID NO: 32 - DNA, 21 bp, Artificial Sequence
  Human
gcacacagagggctacaatgt

SEQ ID NO: 33 - DNA, 19 bp, Artificial Sequence
  Human
cctcccatctccttcatca

SEQ ID NO: 34 - DNA, 18 bp, Artificial Sequence
  Human
accaacccttcccgaggc

SEQ ID NO: 35 - DNA, 20 bp, Artificial Sequence
  Human
ttggttttgtggggcagcat

SEQ ID NO: 36 - DNA, 16 bp, Artificial Sequence
  Human
acggtccgccagaaga

SEQ ID NO: 37 - DNA, 19 bp, Artificial Sequence
  Human
ccacgctgctgaacatgct

SEQ ID NO: 38 - DNA, 20 bp, Artificial Sequence
  Human
tcgaacacctgctggatgac

SEQ ID NO: 39 - DNA, 18 bp, Artificial Sequence
  Human
tctcccccttctcctttg

SEQ ID NO: 40 - DNA, 21 bp, Artificial Sequence
  Human
aggaatcaatgctgctctggg

SEQ ID NO: 41 - DNA, 18 bp, Artificial Sequence
  Human
gtcggagcccttcacggt

SEQ ID NO: 42 - DNA, 16 bp, Artificial Sequence
  Human
ccacctccagttgtcc